SEQUENCE LISTING (153 sequences)

SEQ ID NO: 1 - protein, 6 aa, Homo sapiens
DRGSQS

SEQ ID NO: 2 - protein, 6 aa, Homo sapiens
IYSNGD

SEQ ID NO: 3 - protein, 14 aa, Homo sapiens
CAVNTASGTYKYIF

SEQ ID NO: 4 - protein, 5 aa, Homo sapiens
KGHDR

SEQ ID NO: 5 - protein, 6 aa, Homo sapiens
SFDVKD

SEQ ID NO: 6 - protein, 13 aa, Homo sapiens
CATSDFRGKPQHF

SEQ ID NO: 7 - protein, 113 aa, Homo sapiens
QQKEVEQNSGPLSVPEGAIASLNCTYSDRGSQSFFWYRQYSGKSPELIMSIYSNGDKEDGRFTAQLNKASQYVSLLIRDSQPSDSATYLCAVNTASGTYKYIFGTGTRLKVLA

SEQ ID NO: 8 - protein, 109 aa, Homo sapiens
VTQTPRNRITKTGKRIMLECSQTKGHDRMYWYRQDPGLGLRLIYYSFDVKDINKGEISDGYSVSRQAQAKFSLSLESAIPNQTALYFCATSDFRGKPQHFGDGTRLSIL

SEQ ID NO: 9 - protein, 134 aa, Homo sapiens
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVNTASGTYKYIFGTGTRLKVLA

SEQ ID NO: 10 - protein, 131 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSDFRGKPQHFGDGTRLSIL

SEQ ID NO: 11 - protein, 6 aa, Homo sapiens
DRGSQS

SEQ ID NO: 12 - protein, 6 aa, Homo sapiens
IYSNGD

SEQ ID NO: 13 - protein, 16 aa, Homo sapiens
CAVNRAREAAGNKLTF

SEQ ID NO: 14 - protein, 5 aa, Homo sapiens
MGHDK

SEQ ID NO: 15 - protein, 6 aa, Homo sapiens
SYGVNS

SEQ ID NO: 16 - protein, 13 aa, Homo sapiens
CASSRPGSEKLFF

SEQ ID NO: 17 - protein, 115 aa, Homo sapiens
QQKEVEQNSGPLSVPEGAIASLNCTYSDRGSQSFFWYRQYSGKSPELIMSIYSNGDKEDGRFTAQLNKASQYVSLLIRDSQPSDSATYLCAVNRAREAAGNKLTFGGGTRVLVKP

SEQ ID NO: 18 - protein, 110 aa, Homo sapiens
DIYQTPRYLVIGTGKKITLECSQTMGHDKMYWYQQDPGMELHLIHYSYGVNSTEKGDLSSESTVSRIRTEHFPLTLESARPSHTSQYLCASSRPGSEKLFFGSGTQLSVL

SEQ ID NO: 19 - protein, 136 aa, Homo sapiens
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVNRAREAAGNKLTFGGGTRVLVKP

SEQ ID NO: 20 - protein, 131 aa, synthetic construct
MAIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSRPGSEKLFFGSGTQLSVL

SEQ ID NO: 21 - protein, 5 aa, Homo sapiens
TTSDR

SEQ ID NO: 22 - protein, 7 aa, Homo sapiens
LLSNGAV

SEQ ID NO: 23 - protein, 14 aa, Homo sapiens
CAVDPTGANSKLTF

SEQ ID NO: 24 - protein, 5 aa, Homo sapiens
SGDLS

SEQ ID NO: 25 - protein, 6 aa, Homo sapiens
YYNGEE

SEQ ID NO: 26 - protein, 15 aa, Homo sapiens
CASSVAISGEETQYF

SEQ ID NO: 27 - protein, 112 aa, Homo sapiens
ELKVEQNPLFLSMQEGKNYTIYCNYSTTSDRLYWYRQDPGKSLESLFVLLSNGAVKQEGRLMASLDTKARLSTLHITAAVHDLSATYFCAVDPTGANSKLTFGKGITLSVRP

SEQ ID NO: 28 - protein, 112 aa, Homo sapiens
GVTQTPKHLITATGQRVTLRCSPRSGDLSVYWYQQSLDQGLQFLIQYYNGEERAKGNILERFSAQQFPDLHSELNLSSLELGDSALYFCASSVAISGEETQYFGPGTRLLVL

SEQ ID NO: 29 - protein, 130 aa, Homo sapiens
MKKLLAMILWLQLDRLSGELKVEQNPLFLSMQEGKNYTIYCNYSTTSDRLYWYRQDPGKSLESLFVLLSNGAVKQEGRLMASLDTKARLSTLHITAAVHDLSATYFCAVDPTGANSKLTFGKGITLSVRP

SEQ ID NO: 30 - protein, 133 aa, synthetic construct
MAFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVAISGEETQYFGPGTRLLVL

SEQ ID NO: 31 - protein, 6 aa, Homo sapiens
DRGSQS

SEQ ID NO: 32 - protein, 6 aa, Homo sapiens
IYSNGD

SEQ ID NO: 33 - protein, 9 aa, Homo sapiens
CAVKSWVIF

SEQ ID NO: 34 - protein, 5 aa, Homo sapiens
SGHKS

SEQ ID NO: 35 - protein, 6 aa, Homo sapiens
YYEKEE

SEQ ID NO: 36 - protein, 15 aa, Homo sapiens
CASSPSAQSSYGYTF

SEQ ID NO: 37 - protein, 108 aa, Homo sapiens
QQKEVEQNSGPLSVPEGAIASLNCTYSDRGSQSFFWYRQYSGKSPELIMFIYSNGDKEDGRFTAQLNKASQYVSLLIRDSQPSDSATYLCAVKSWVIFGKGTRLHILP

SEQ ID NO: 38 - protein, 112 aa, Homo sapiens
GVTQSPTHLIKTRGQQVTLRCSPISGHKSVSWYQQVLGQGPQFIFQYYEKEERGRGNFPDRFSARQFPNYSSELNVNALLLGDSALYLCASSPSAQSSYGYTFGSGTRLTVV

SEQ ID NO: 39 - protein, 128 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKSWVIFGKGTRLHILP

SEQ ID NO: 40 - protein, 133 aa, synthetic construct
MAPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASSPSAQSSYGYTFGSGTRLTVV

SEQ ID NO: 41 - protein, 6 aa, Homo sapiens
DRGSQS

SEQ ID NO: 42 - protein, 6 aa, Homo sapiens
IYSNGD

SEQ ID NO: 43 - protein, 13 aa, Homo sapiens
CAVPNSGYSTLTF

SEQ ID NO: 44 - protein, 5 aa, Homo sapiens
SGHKS

SEQ ID NO: 45 - protein, 6 aa, Homo sapiens
YYEKEE

SEQ ID NO: 46 - protein, 17 aa, Homo sapiens
CASRTGTVTNSYNEQFF

SEQ ID NO: 47 - protein, 112 aa, Homo sapiens
QQKEVEQNSGPLSVPEGAIASLNCTYSDRGSQSFFWYRQYSGKSPELIMFIYSNGDKEDGRFTAQLNKASQYVSLLIRDSQPSDSATYLCAVPNSGYSTLTFGKGTMLLVSP

SEQ ID NO: 48 - protein, 114 aa, Homo sapiens
GVTQSPTHLIKTRGQQVTLRCSPISGHKSVSWYQQVLGQGPQFIFQYYEKEERGRGNFPDRFSARQFPNYSSELNVNALLLGDSALYLCASRTGTVTNSYNEQFFGPGTRLTVL

SEQ ID NO: 49 - protein, 132 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVPNSGYSTLTFGKGTMLLVSP

SEQ ID NO: 50 - protein, 135 aa, synthetic construct
MAPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASRTGTVTNSYNEQFFGPGTRLTVL

SEQ ID NO: 51 - protein, 6 aa, Homo sapiens
YGGTVN

SEQ ID NO: 52 - protein, 8 aa, Homo sapiens
YFSGDPLV

SEQ ID NO: 53 - protein, 13 aa, Homo sapiens
CAVKDTGFQKLVF

SEQ ID NO: 54 - protein, 6 aa, Homo sapiens
DFQATT

SEQ ID NO: 55 - protein, 7 aa, Homo sapiens
SNEGSKA

SEQ ID NO: 56 - protein, 14 aa, Homo sapiens
CSARGGRNSFEQFF

SEQ ID NO: 57 - protein, 112 aa, Homo sapiens
QSVSQHNHHVILSEAASLELGCNYSYGGTVNLFWYVQYPGQHLQLLLKYFSGDPLVKGIKGFEAEFIKSKFSFNLRKPSVQWSDTAEYFCAVKDTGFQKLVFGTGTRLLVSP

SEQ ID NO: 58 - protein, 120 aa, Homo sapiens
GSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARGGRNSFEQFFGPGTRLTVL

SEQ ID NO: 59 - protein, 132 aa, Homo sapiens
MLLLLIPVLGMIFALRDARAQSVSQHNHHVILSEAASLELGCNYSYGGTVNLFWYVQYPGQHLQLLLKYFSGDPLVKGIKGFEAEFIKSKFSFNLRKPSVQWSDTAEYFCAVKDTGFQKLVFGTGTRLLVSP

SEQ ID NO: 60 - protein, 141 aa, Homo sapiens
MLLLLLLLGPGISLLLPGSLAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARGGRNSFEQFFGPGTRLTVL

SEQ ID NO: 61 - protein, 6 aa, Homo sapiens
YGGTVN

SEQ ID NO: 62 - protein, 8 aa, Homo sapiens
YFSGDPLV

SEQ ID NO: 63 - protein, 13 aa, Homo sapiens
CAVKDTGFQKLVF

SEQ ID NO: 64 - protein, 6 aa, Homo sapiens
DFQATT

SEQ ID NO: 65 - protein, 7 aa, Homo sapiens
SNEGSKA

SEQ ID NO: 66 - protein, 14 aa, Homo sapiens
CSARGGARSYEQYF

SEQ ID NO: 67 - protein, 112 aa, Homo sapiens; identical to SEQ ID NO: 57

SEQ ID NO: 68 - protein, 120 aa, Homo sapiens
GSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARGGARSYEQYFGPGTRLTVT

SEQ ID NO: 69 - protein, 132 aa, Homo sapiens; identical to SEQ ID NO: 59

SEQ ID NO: 70 - protein, 141 aa, Homo sapiens
MLLLLLLLGPGISLLLPGSLAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARGGARSYEQYFGPGTRLTVT

SEQ ID NO: 71 - protein, 137 aa, synthetic construct
  VARIANT 48: note = X at position 48 is T or C
  VARIANT 112: note = X at position 112 is S, A, V, L, I, P, F, M, or W
  VARIANT 114: note = X at position 114 is M, A, V, L, I, P, F, or W
  VARIANT 115: note = X at position 115 is G, A, V, L, I, P, F, M, or W
NIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 72 - protein, 173 aa, synthetic construct
  VARIANT 57: note = X at position 57 is S or C
EDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 73 - protein, 137 aa, synthetic construct
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 74 - protein, 173 aa, synthetic construct
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 75 - protein, 137 aa, Mus musculus
NIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 76 - protein, 173 aa, Mus musculus
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 77 - protein, 271 aa, synthetic construct
  VARIANT 182: note = X at position 182 is T or C
  VARIANT 246: note = X at position 246 is S, A, V, L, I, P, F, M, or W
  VARIANT 248: note = X at position 248 is M, A, V, L, I, P, F, or W
  VARIANT 249: note = X at position 249 is G, A, V, L, I, P, F, M, or W
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVNTASGTYKYIFGTGTRLKVLANIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 78 - protein, 304 aa, synthetic construct
  VARIANT 188: note = X at position 188 is S or C
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSDFRGKPQHFGDGTRLSILEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 79 - protein, 250 aa, synthetic construct
  VARIANT 161: note = X at position 161 is T or C
  VARIANT 225: note = X at position 225 is S, A, V, L, I, P, F, M, or W
  VARIANT 227: note = X at position 227 is M, A, V, L, I, P, F, or W
  VARIANT 228: note = X at position 228 is G, A, V, L, I, P, F, M, or W
QQKEVEQNSGPLSVPEGAIASLNCTYSDRGSQSFFWYRQYSGKSPELIMSIYSNGDKEDGRFTAQLNKASQYVSLLIRDSQPSDSATYLCAVNTASGTYKYIFGTGTRLKVLANIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 80 - protein, 282 aa, synthetic construct
  VARIANT 166: note = X at position 166 is S or C
VTQTPRNRITKTGKRIMLECSQTKGHDRMYWYRQDPGLGLRLIYYSFDVKDINKGEISDGYSVSRQAQAKFSLSLESAIPNQTALYFCATSDFRGKPQHFGDGTRLSILEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 81 - protein, 271 aa, synthetic construct
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVNTASGTYKYIFGTGTRLKVLANIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 82 - protein, 304 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSDFRGKPQHFGDGTRLSILEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 83 - protein, 250 aa, synthetic construct
QQKEVEQNSGPLSVPEGAIASLNCTYSDRGSQSFFWYRQYSGKSPELIMSIYSNGDKEDGRFTAQLNKASQYVSLLIRDSQPSDSATYLCAVNTASGTYKYIFGTGTRLKVLANIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 84 - protein, 282 aa, synthetic construct
VTQTPRNRITKTGKRIMLECSQTKGHDRMYWYRQDPGLGLRLIYYSFDVKDINKGEISDGYSVSRQAQAKFSLSLESAIPNQTALYFCATSDFRGKPQHFGDGTRLSILEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 85 - protein, 273 aa, synthetic construct
  VARIANT 184: note = X at position 184 is T or C
  VARIANT 248: note = X at position 248 is S, A, V, L, I, P, F, M, or W
  VARIANT 250: note = X at position 250 is M, A, V, L, I, P, F, or W
  VARIANT 251: note = X at position 251 is G, A, V, L, I, P, F, M, or W
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVNRAREAAGNKLTFGGGTRVLVKPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 86 - protein, 304 aa, synthetic construct
  VARIANT 188: note = X at position 188 is S or C
MAIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSRPGSEKLFFGSGTQLSVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 87 - protein, 252 aa, synthetic construct
  VARIANT 163: note = X at position 163 is T or C
  VARIANT 227: note = X at position 227 is S, A, V, L, I, P, F, M, or W
  VARIANT 229: note = X at position 229 is M, A, V, L, I, P, F, or W
  VARIANT 230: note = X at position 230 is G, A, V, L, I, P, F, M, or W
QQKEVEQNSGPLSVPEGAIASLNCTYSDRGSQSFFWYRQYSGKSPELIMSIYSNGDKEDGRFTAQLNKASQYVSLLIRDSQPSDSATYLCAVNRAREAAGNKLTFGGGTRVLVKPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 88 - protein, 283 aa, synthetic construct
  VARIANT 167: note = X at position 167 is S or C
DIYQTPRYLVIGTGKKITLECSQTMGHDKMYWYQQDPGMELHLIHYSYGVNSTEKGDLSSESTVSRIRTEHFPLTLESARPSHTSQYLCASSRPGSEKLFFGSGTQLSVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 89 - protein, 273 aa, synthetic construct
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVNRAREAAGNKLTFGGGTRVLVKPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 90 - protein, 304 aa, synthetic construct
MAIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSRPGSEKLFFGSGTQLSVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 91 - protein, 252 aa, synthetic construct
QQKEVEQNSGPLSVPEGAIASLNCTYSDRGSQSFFWYRQYSGKSPELIMSIYSNGDKEDGRFTAQLNKASQYVSLLIRDSQPSDSATYLCAVNRAREAAGNKLTFGGGTRVLVKPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 92 - protein, 283 aa, synthetic construct
DIYQTPRYLVIGTGKKITLECSQTMGHDKMYWYQQDPGMELHLIHYSYGVNSTEKGDLSSESTVSRIRTEHFPLTLESARPSHTSQYLCASSRPGSEKLFFGSGTQLSVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 93 - protein, 267 aa, synthetic construct
  VARIANT 178: note = X at position 178 is T or C
  VARIANT 242: note = X at position 242 is S, A, V, L, I, P, F, M, or W
  VARIANT 244: note = X at position 244 is M, A, V, L, I, P, F, or W
  VARIANT 245: note = X at position 245 is G, A, V, L, I, P, F, M, or W
MKKLLAMILWLQLDRLSGELKVEQNPLFLSMQEGKNYTIYCNYSTTSDRLYWYRQDPGKSLESLFVLLSNGAVKQEGRLMASLDTKARLSTLHITAAVHDLSATYFCAVDPTGANSKLTFGKGITLSVRPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 94 - protein, 306 aa, synthetic construct
  VARIANT 190: note = X at position 190 is S or C
MAFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVAISGEETQYFGPGTRLLVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 95 - protein, 249 aa, synthetic construct
  VARIANT 160: note = X at position 160 is T or C
  VARIANT 224: note = X at position 224 is S, A, V, L, I, P, F, M, or W
  VARIANT 226: note = X at position 226 is M, A, V, L, I, P, F, or W
  VARIANT 227: note = X at position 227 is G, A, V, L, I, P, F, M, or W
ELKVEQNPLFLSMQEGKNYTIYCNYSTTSDRLYWYRQDPGKSLESLFVLLSNGAVKQEGRLMASLDTKARLSTLHITAAVHDLSATYFCAVDPTGANSKLTFGKGITLSVRPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 96 - protein, 285 aa, synthetic construct
  VARIANT 169: note = X at position 169 is S or C
GVTQTPKHLITATGQRVTLRCSPRSGDLSVYWYQQSLDQGLQFLIQYYNGEERAKGNILERFSAQQFPDLHSELNLSSLELGDSALYFCASSVAISGEETQYFGPGTRLLVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 97 - protein, 267 aa, synthetic construct
MKKLLAMILWLQLDRLSGELKVEQNPLFLSMQEGKNYTIYCNYSTTSDRLYWYRQDPGKSLESLFVLLSNGAVKQEGRLMASLDTKARLSTLHITAAVHDLSATYFCAVDPTGANSKLTFGKGITLSVRPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 98 - protein, 306 aa, synthetic construct
MAFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVAISGEETQYFGPGTRLLVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 99 - protein, 249 aa, synthetic construct
ELKVEQNPLFLSMQEGKNYTIYCNYSTTSDRLYWYRQDPGKSLESLFVLLSNGAVKQEGRLMASLDTKARLSTLHITAAVHDLSATYFCAVDPTGANSKLTFGKGITLSVRPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 100 - protein, 285 aa, synthetic construct
GVTQTPKHLITATGQRVTLRCSPRSGDLSVYWYQQSLDQGLQFLIQYYNGEERAKGNILERFSAQQFPDLHSELNLSSLELGDSALYFCASSVAISGEETQYFGPGTRLLVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 101 - protein, 265 aa, synthetic construct
  VARIANT 176: note = X at position 176 is T or C
  VARIANT 240: note = X at position 240 is S, A, V, L, I, P, F, M, or W
  VARIANT 242: note = X at position 242 is M, A, V, L, I, P, F, or W
  VARIANT 243: note = X at position 243 is G, A, V, L, I, P, F, M, or W
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKSWVIFGKGTRLHILPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 102 - protein, 306 aa, synthetic construct
  VARIANT 190: note = X at position 190 is S or C
MAPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASSPSAQSSYGYTFGSGTRLTVVEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 103 - protein, 245 aa, synthetic construct
  VARIANT 156: note = X at position 156 is T or C
  VARIANT 220: note = X at position 220 is S, A, V, L, I, P, F, M, or W
  VARIANT 222: note = X at position 222 is M, A, V, L, I, P, F, or W
  VARIANT 223: note = X at position 223 is G, A, V, L, I, P, F, M, or W
QQKEVEQNSGPLSVPEGAIASLNCTYSDRGSQSFFWYRQYSGKSPELIMFIYSNGDKEDGRFTAQLNKASQYVSLLIRDSQPSDSATYLCAVKSWVIFGKGTRLHILPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 104 - protein, 285 aa, synthetic construct
  VARIANT 169: note = X at position 169 is S or C
GVTQSPTHLIKTRGQQVTLRCSPISGHKSVSWYQQVLGQGPQFIFQYYEKEERGRGNFPDRFSARQFPNYSSELNVNALLLGDSALYLCASSPSAQSSYGYTFGSGTRLTVVEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 105 - protein, 265 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKSWVIFGKGTRLHILPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 106 - protein, 306 aa, synthetic construct
MAPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASSPSAQSSYGYTFGSGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 107 - protein, 245 aa, synthetic construct
QQKEVEQNSGPLSVPEGAIASLNCTYSDRGSQSFFWYRQYSGKSPELIMFIYSNGDKEDGRFTAQLNKASQYVSLLIRDSQPSDSATYLCAVKSWVIFGKGTRLHILPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 108 - protein, 285 aa, synthetic construct
GVTQSPTHLIKTRGQQVTLRCSPISGHKSVSWYQQVLGQGPQFIFQYYEKEERGRGNFPDRFSARQFPNYSSELNVNALLLGDSALYLCASSPSAQSSYGYTFGSGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 109 - protein, 269 aa, synthetic construct
  VARIANT 180: note = X at position 180 is T or C
  VARIANT 244: note = X at position 244 is S, A, V, L, I, P, F, M, or W
  VARIANT 246: note = X at position 246 is M, A, V, L, I, P, F, or W
  VARIANT 247: note = X at position 247 is G, A, V, L, I, P, F, M, or W
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVPNSGYSTLTFGKGTMLLVSPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 110 - protein, 308 aa, synthetic construct
  VARIANT 192: note = X at position 192 is S or C
MAPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASRTGTVTNSYNEQFFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 111 - protein, 249 aa, synthetic construct
  VARIANT 160: note = X at position 160 is T or C
  VARIANT 224: note = X at position 224 is S, A, V, L, I, P, F, M, or W
  VARIANT 226: note = X at position 226 is M, A, V, L, I, P, F, or W
  VARIANT 227: note = X at position 227 is G, A, V, L, I, P, F, M, or W
QQKEVEQNSGPLSVPEGAIASLNCTYSDRGSQSFFWYRQYSGKSPELIMFIYSNGDKEDGRFTAQLNKASQYVSLLIRDSQPSDSATYLCAVPNSGYSTLTFGKGTMLLVSPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 112 - protein, 287 aa, synthetic construct
  VARIANT 171: note = X at position 171 is S or C
GVTQSPTHLIKTRGQQVTLRCSPISGHKSVSWYQQVLGQGPQFIFQYYEKEERGRGNFPDRFSARQFPNYSSELNVNALLLGDSALYLCASRTGTVTNSYNEQFFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 113 - protein, 269 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVPNSGYSTLTFGKGTMLLVSPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 114 - protein, 308 aa, synthetic construct
MAPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASRTGTVTNSYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 115 - protein, 249 aa, synthetic construct
QQKEVEQNSGPLSVPEGAIASLNCTYSDRGSQSFFWYRQYSGKSPELIMFIYSNGDKEDGRFTAQLNKASQYVSLLIRDSQPSDSATYLCAVPNSGYSTLTFGKGTMLLVSPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 116 - protein, 287 aa, synthetic construct
GVTQSPTHLIKTRGQQVTLRCSPISGHKSVSWYQQVLGQGPQFIFQYYEKEERGRGNFPDRFSARQFPNYSSELNVNALLLGDSALYLCASRTGTVTNSYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 117 - protein, 269 aa, synthetic construct
  VARIANT 180: note = X at position 180 is T or C
  VARIANT 244: note = X at position 244 is S, A, V, L, I, P, F, M, or W
  VARIANT 246: note = X at position 246 is M, A, V, L, I, P, F, or W
  VARIANT 247: note = X at position 247 is G, A, V, L, I, P, F, M, or W
MLLLLIPVLGMIFALRDARAQSVSQHNHHVILSEAASLELGCNYSYGGTVNLFWYVQYPGQHLQLLLKYFSGDPLVKGIKGFEAEFIKSKFSFNLRKPSVQWSDTAEYFCAVKDTGFQKLVFGTGTRLLVSPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 118 - protein, 314 aa, synthetic construct
  VARIANT 198: note = X at position 198 is S or C
MLLLLLLLGPGISLLLPGSLAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARGGRNSFEQFFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 119 - protein, 249 aa, synthetic construct
  VARIANT 160: note = X at position 160 is T or C
  VARIANT 224: note = X at position 224 is S, A, V, L, I, P, F, M, or W
  VARIANT 226: note = X at position 226 is M, A, V, L, I, P, F, or W
  VARIANT 227: note = X at position 227 is G, A, V, L, I, P, F, M, or W
QSVSQHNHHVILSEAASLELGCNYSYGGTVNLFWYVQYPGQHLQLLLKYFSGDPLVKGIKGFEAEFIKSKFSFNLRKPSVQWSDTAEYFCAVKDTGFQKLVFGTGTRLLVSPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 120 - protein, 293 aa, synthetic construct
  VARIANT 177: note = X at position 177 is S or C
GSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARGGRNSFEQFFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 121 - protein, 269 aa, synthetic construct
MLLLLIPVLGMIFALRDARAQSVSQHNHHVILSEAASLELGCNYSYGGTVNLFWYVQYPGQHLQLLLKYFSGDPLVKGIKGFEAEFIKSKFSFNLRKPSVQWSDTAEYFCAVKDTGFQKLVFGTGTRLLVSPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 122 - protein, 314 aa, synthetic construct
MLLLLLLLGPGISLLLPGSLAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARGGRNSFEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 123 - protein, 249 aa, synthetic construct
QSVSQHNHHVILSEAASLELGCNYSYGGTVNLFWYVQYPGQHLQLLLKYFSGDPLVKGIKGFEAEFIKSKFSFNLRKPSVQWSDTAEYFCAVKDTGFQKLVFGTGTRLLVSPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 124 - protein, 293 aa, synthetic construct
GSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARGGRNSFEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 125 - protein, 269 aa, synthetic construct; identical to SEQ ID NO: 117
  VARIANT 180: note = X at position 180 is T or C
  VARIANT 244: note = X at position 244 is S, A, V, L, I, P, F, M, or W
  VARIANT 246: note = X at position 246 is M, A, V, L, I, P, F, or W
  VARIANT 247: note = X at position 247 is G, A, V, L, I, P, F, M, or W

SEQ ID NO: 126 - protein, 314 aa, synthetic construct
  VARIANT 198: note = X at position 198 is S or C
MLLLLLLLGPGISLLLPGSLAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARGGARSYEQYFGPGTRLTVTEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 127 - protein, 249 aa, synthetic construct; identical to SEQ ID NO: 119
  VARIANT 160: note = X at position 160 is T or C
  VARIANT 224: note = X at position 224 is S, A, V, L, I, P, F, M, or W
  VARIANT 226: note = X at position 226 is M, A, V, L, I, P, F, or W
  VARIANT 227: note = X at position 227 is G, A, V, L, I, P, F, M, or W

SEQ ID NO: 128 - protein, 293 aa, synthetic construct
  VARIANT 177: note = X at position 177 is S or C
GSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARGGARSYEQYFGPGTRLTVTEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 129 - protein, 269 aa, synthetic construct; identical to SEQ ID NO: 121

SEQ ID NO: 130 - protein, 314 aa, synthetic construct
MLLLLLLLGPGISLLLPGSLAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARGGARSYEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 131 - protein, 249 aa, synthetic construct; identical to SEQ ID NO: 123

SEQ ID NO: 132 - protein, 293 aa, synthetic construct
GSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARGGARSYEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 133 - protein, 27 aa, synthetic construct
RAKRSGSGATNFSLLKQAGDVEENPGP

SEQ ID NO: 134 - protein, 602 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSDFRGKPQHFGDGTRLSILEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVNTASGTYKYIFGTGTRLKVLANIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 135 - protein, 604 aa, synthetic construct
MAIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSRPGSEKLFFGSGTQLSVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVNRAREAAGNKLTFGGGTRVLVKPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 136 - protein, 600 aa, synthetic construct
MAFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVAISGEETQYFGPGTRLLVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMKKLLAMILWLQLDRLSGELKVEQNPLFLSMQEGKNYTIYCNYSTTSDRLYWYRQDPGKSLESLFVLLSNGAVKQEGRLMASLDTKARLSTLHITAAVHDLSATYFCAVDPTGANSKLTFGKGITLSVRPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 137 - protein, 597 aa, synthetic construct
MAPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASSPSAQSSYGYTFGSGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKSWVIFGKGTRLHILPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 138 - protein, 603 aa, synthetic construct
MAPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASRTGTVTNSYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVPNSGYSTLTFGKGTMLLVSPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 139 - protein, 609 aa, synthetic construct
MLLLLLLLGPGISLLLPGSLAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARGGRNSFEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGMLLLLIPVLGMIFALRDARAQSVSQHNHHVILSEAASLELGCNYSYGGTVNLFWYVQYPGQHLQLLLKYFSGDPLVKGIKGFEAEFIKSKFSFNLRKPSVQWSDTAEYFCAVKDTGFQKLVFGTGTRLLVSPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 140 - protein, 609 aa, synthetic construct
MLLLLLLLGPGISLLLPGSLAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARGGARSYEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGMLLLLIPVLGMIFALRDARAQSVSQHNHHVILSEAASLELGCNYSYGGTVNLFWYVQYPGQHLQLLLKYFSGDPLVKGIKGFEAEFIKSKFSFNLRKPSVQWSDTAEYFCAVKDTGFQKLVFGTGTRLLVSPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 141 - protein, 9 aa, Homo sapiens
AVCPWTWLR

SEQ ID NO: 142 - protein, 9 aa, Homo sapiens
LLVDLAEEL

SEQ ID NO: 143 - protein, 10 aa, Homo sapiens
RLLVDLAEEL

SEQ ID NO: 144 - protein, 9 aa, Homo sapiens
LPVDLAEEL

SEQ ID NO: 145 - protein, 10 aa, Homo sapiens
RLPVDLAEEL

SEQ ID NO: 146 - protein, 26 aa, synthetic construct
RAKRSGSGATNFSLLKQAGDVEENPG

SEQ ID NO: 147 - protein, 156 aa, Homo sapiens
MEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPD

SEQ ID NO: 148 - protein, 132 aa, Homo sapiens
MVRRFLVTLRIRRACGPPRVRVFVVHIPRLTGEWAAPGAPAAVALVLMLLRSQRLGQQPLPRRPGHDDGQRPSGGAAAAPRRGAQLRRPRHSHPTRARRCPGGLPGHAGGAAPGRGAAGRARCLGPSARGPG

SEQ ID NO: 149 - protein, 156 aa, Homo sapiens
MEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLLVDLAEELGHRDVARYLRAAAGGTRGSNHARIDAAEGPSDIPD

SEQ ID NO: 150 - protein, 170 aa, Homo sapiens
MVRRFLVTLRIRRACGPPRVRVFVVHIPRLTGEWAAPGAPAAVALVLMLLRSQRLGQQPLPRRPGHDDGQRPSGGAAAAPRRGAQLRRPRHSHPTRARRCPGGLPGHAGGAAPGRARLDVRDAWGRLPVDLAEELGHRDVARYLRAAAGGTRGSNHARIDAAEGPSDIPD

SEQ ID NO: 151 - protein, 144 aa, Homo sapiens
MEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGRGWTCAMPGAVCPWTWLRSWAIAMSHGTCARLRGAPEAVTMPA

SEQ ID NO: 152 - protein, 158 aa, Homo sapiens
MVRRFLVTLRIRRACGPPRVRVFVVHIPRLTGEWAAPGAPAAVALVLMLLRSQRLGQQPLPRRPGHDDGQRPSWSCCCSTARSPTAPTPPLSPDPCTTLPGRASWTRWWCCTGPGRGWTCAMPGAVCPWTWLRSWAIAMSHGTCARLRGAPEAVTMPA

SEQ ID NO: 153 - protein, 117 aa, Homo sapiens
MEPAAGSSMEPSADWLATAAARGRVEEVRALLEAGALPNAPNSYGRRPIQVMMMGSARVGAAAAPRRGAQLRRPRHSHPTRARRCPGGLPGHAGGAAPGRGAAGRARCLGPSARGPG

(19) United States
(12) Patent Application Publication
(10) Pub. No.: US 2026/0146073 A1
(43) Pub. Date: May 28, 2026

(54) T CELL RECEPTORS TARGETING MUTATED CDKN2A

(71) Applicant: The United States of America, as represented by the Secretary, Department of Health and Human Servic, Bethesda, MD (US)

(72) Inventors: Sri Krishna, Bethesda, MD (US); Shoshana T. Levi, Boston, MA (US); Rami Yoseph, Gaithersburg, MD (US); Frank J. Lowery, III, Clarksburg, MD (US); Shirley K. Nah, Washington, DC (US); Paul F. Robbins, Chevy Chase, MD (US); Steven A. Rosenberg, Potomac, MD (US)

(73) Assignee: The United States of America, as represented by the Secretary, Department of Health and Human Servic, Bethesda, MD (US)

(21) Appl. No.: 19/118,202

(22) PCT Filed: Oct. 30, 2023
(86) PCT No.: PCT/US2023/078190

§ 371 Date: Apr. 3, 2025

Related U.S. Application Data
(60) Provisional application No. 63/381,591, filed on Oct. 31, 2022.

(51) Int. Cl. C07K 14/725; A61K 40/11; A61K 40/32; A61K 40/42; A61P 35/00; G01N 33/575

(52) U.S. Cl. CPC C07K 14/7051; A61K 40/11; A61K 40/32; A61K 40/42; A61P 35/00; G01N 33/5758; C07K 2319/00; G01N 2333/7051

(57) ABSTRACT

Disclosed is an isolated or purified T cell receptor (TCR), wherein the TCR has antigenic specificity for a mutated amino acid sequence encoded by mutated CDKN2A. The TCRs may recognize mutated CDKN2A peptide presented by an HLA-A molecule. Related polypeptides and proteins, as well as related nucleic acids, recombinant expression vectors, host cells, populations of cells, and pharmaceutical compositions are also provided. Also disclosed are methods of detecting the presence of cancer in a mammal and methods of treating or preventing cancer in a mammal.

### CROSS-REFERENCE TO RELATED APPLICATION

This patent application claims the benefit of U.S. Provisional Patent Application No, 63/381,591, filed Oct., 31, 2022, which is incorporated by reference in its entirety herein.

### STATEMENT REGARDING FEDERALLY SPONSORED RESEARCH OR DEVELOPMENT

This invention was made with Government support under project number ZIABC010984 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 187,155 Byte XML file named “768421_ST26.XML.” dated Oct. 27, 2023.

### BACKGROUND OF THE INVENTION

Some cancers may have verylimited treatment options, particularly when the cancer becomes metastatic and unresectable. Despite advances in treatments such as, for example, surgery, chemotherapy, and radiation therapy, the prognosis for many cancers, such as, for example, melanoma and pancreatic and gastrointestinal cancers, may be poor. Accordingly, there exists an unmet need for additional treatments for cancer.

### BRIEF SUMMARY OF THE INVENTION

An aspect of the invention provides an isolated or purified T-cell receptor (TCR) having antigenic specificity for a mutated amino acid sequence encoded by mutated cyclin-dependent kinase inhibitor 2A (CDKN2A), wherein the TCR comprises the amino acid sequence(s) of: (a) all of SEQ ID NOs: 1-3: (b) all of SEQ ID NOs: 4-6: (c) all of SEQ ID NOs: 1-6: (d) all of SEQ ID NOs: 11-13: (e) all of SEQ ID NOs: 14-16: (f) all of SEQ ID NOs: 11-16; (g) all of SEQ ID NOs: 21-23: (h) all of SEQ ID NOs: 24-26: (i) all of SEQ ID NOs: 21-26: (i) all of SEQ ID NO: 31-33: (k) all of SEQ ID NO: 34-36: (1) all of SEQ ID NO: 31-36: (m) all of SEQ ID NO: 41-43: (n) all of SEQ ID NO: 44-46: (0) all of SEQ ID NO: 41-46: (p) all of SEQ ID NO: 51-53; (q) all of SEQ ID NO: 54-56; (r) all of SEQ ID NO: 51-56: (s) all of SEQ ID NO: 61-63: (t) all of SEQ ID NO: 64-66; or (u) all of SEQ ID NO: 61-66.

Another aspect of the invention provides an isolated or purified polypeptide comprising a functional portion of the inventive TCR, wherein the functional portion comprises the amino acid sequences of: (a) all of SEQ ID NOs: 1-3: (b) all of SEQ ID NOs: 4-6: (c) all of SEQ ID NOs: 1-6: (d) all of SEQ ID NOs: 11-13: (e) all of SEQ ID NOs: 14-16: (f) all of SEQ ID NOs: 11-16: (g) all of SEQ ID NOs: 21-23: (h) all of SEQ ID NOs: 24-26; (i) all of SEQ ID NOs: 21-26: (j) all of SEQ ID NO: 31-33: (k) all of SEQ ID NO: 34-36: (1) all of SEQ ID NO: 31-36: (m) all of SEQ ID NO: 41-43: (n) all of SEQ ID NO: 44-46: (0) all of SEQ ID NO: 41-46: (p) all of SEQ ID NO: 51-53; (q) all of SEQ ID NO: 54-56; (r) all of SEQ ID NO: 51-56: (s) all of SEQ ID NO: 61-63: (t) all of SEQ ID NO: 64-66; or (u) all of SEQ ID NO: 61-66.

Still another aspect of the invention provides an isolated or purified protein. comprising: (a) a first polypeptide chain comprising the amino acid sequences of SEQ ID NOs: 1-3 and a second polypeptide chain comprising the amino acid sequences of SEQ ID NOs: 4-6: (b) a first polypeptide chain comprising the amino acid sequences of SEQ ID NOs: 11-13 and a second polypeptide chain comprising the amino acid sequences of SEQ ID NOS: 14-16: (c) a first polypeptide chain comprising the amino acid sequences of SEQ ID NOs: 21-23 and a second polypeptide chain comprising the amino acid sequences of SEQ ID NOs: 24-26: (d) a first polypeptide chain comprising the amino acid sequences of SEQ ID NOs: 31-33 and a second polypeptide chain comprising the amino acid sequences of SEQ ID NOs: 34-36; (e) a first polypeptide chain comprising the amino acid sequences of SEQ ID NOs: 41-43 and a second polypeptide chain comprising the amino acid sequences of SEQ ID NOs: 44-46: (f) a first polypeptide chain comprising the amino acid sequences of SEQ ID NOs: 51-53 and a second polypeptide chain comprising the amino acid sequences of SEQ ID NOs: 54-56; or (g) a first polypeptide chain comprising the amino acid sequences of SEQ ID NOs: 61-63 and a second polypeptide chain comprising the amino acid sequences of SEQ ID NOs: 64-66.

Another aspect of the invention provides a bispecific engager TCR fusion protein comprising (i) any of the inventive TCRs, polypeptides, or proteins described herein and (ii) an anti-CD3 engager.

Further aspects of the invention provide related nucleic acids, recombinant expression vectors, host cells, populations of cells, and pharmaceutical compositions relating to the inventive TCRs, polypeptides, and proteins.

An aspect of the invention provides an isolated or purified nucleic acid comprising, from 5′ to 3′, a first nucleic acid sequence and a second nucleotide sequence, wherein the first and second nucleotide sequence, respectively, encode the amino sequences of SEQ ID NOs: 7 and 8:8 and 7; 9 and 10; 10 and 9; 17 and 18; 18 and 17; 19 and 20; 20 and 19; 27 and 28; 28 and 27; 29 and 30; 30 and 29; 37 and 38; 38 and 37; 39 and 40; 40 and 39; 47 and 48; 48 and 47; 49 and 50; 50 and 49; 57 and 58; 58 and 57; 59 and 60; 60 and 59; 67 and 68; 68 and 67; 69 and 70; 70 and 69; 77 and 78; 78 and 77; 79 and 80; 80 and 79; 81 and 82; 82 and 81; 83 and 84; 84 and 83; 85 and 86; 86 and 85; 87 and 88; 88 and 87; 89 and 90; 90 and 89; 91 and 92; 92 and 91; 93 and 94; 94 and 93; 95 and 96; 96 and 95; 97 and 98; 98 and 97; 99 and 100; 100 and 99; 101 and 102; 102 and 101; 103 and 104; 104 and 103; 105 and 106; 106 and 105; 107 and 108; 108 and 107; 109 and 110; 111 and 112; 112 and 111; 113 and 114; 114 and 113; 115 and 116; 116 and 115; 117 and 118; 118 and 117; 119 and 120; 120 and 119; 121 and 122; 122 and 121; 123 and 124; 124 and 123; 125 and 126; 126 and 125; 127 and 128; 128 and 127; 129 and 130; 130 and 129; 131 and 132; or 132 and 131.

Another aspect of the invention provides a method of producing a host cell expressing a TCR that has antigenic specificity for the peptide of AVCPWTWLR (SEQ ID NO: 141), LLVDLAEEL (SEQ ID NO: 142), or RLLVDLAEEL (SEQ ID NO: 143), the method comprising contacting a cell with the inventive recombinant expression vector under conditions that allow introduction of the recombinant expression vector into the cell.

Still another aspect of the invention provides a method of producing the inventive TCR, polypeptide, or protein, the method comprising culturing the inventive host cell or the population of host cells so that the inventive TCR, polypeptide, or protein is produced.

Another aspect of the invention provides a method of detecting the presence of cancer in mammal, the method comprising: (a) contacting a sample comprising cells of the cancer with the inventive TCR, polypeptide, protein, nucleic acid, recombinant expression vector, host cell, population, or pharmaceutical composition, thereby forming a complex; and (b) detecting the complex, wherein detection of the complex is indicative of the presence of cancer in the mammal.

Another aspect of the invention provides a method of inducing an immune response against cancer in a mammal, the method comprising administering to the mammal the inventive TCR, polypeptide, protein, nucleic acid, recombinant expression vector, host cell, population, or pharmaceutical composition, in an amount effective to induce the immune response against cancer in the mammal.

Another aspect of the invention provides a method of treating or preventing cancer in a mammal, the method comprising administering to the mammal the inventive TCR, polypeptide, protein, nucleic acid, recombinant expression vector, host cell, population, or pharmaceutical composition in an amount effective to treat or prevent cancer in the mammal.

### BRIEF DESCRIPTION OF THE SEVERAL VIEWS OF THE DRAWINGS

FIG. 1 shows graphs depicting experimental data (dot plots) illustrating the percentage of TIL from Patient 3333, which had been previously determined to recognize autologous tumor, which bound to the CDKN2A frameshift mutated peptide AVCPWTWLR (SEQ ID NO: 141) tetramer conjugated to APC or PE (right plot: TET+4.09) or conjugated to no fluorophore (left plot: TET+8.76×10⁴).

FIG. 2 is a graph showing the percentage of effector cells (positive for expression of the murine TCR constant region (mTCR) and CD8) that upregulated 4-1BB expression following co-culture with target cells. The effector cells were healthy donor PBL independently transduced with a retroviral vector encoding the 3333 TCR-A, 3333 TCR-C, or 1913 TCR-41BB. Target cells were Cos7 cells independently transfected with HLA-A*03:01 or HLA-A*11:01 and pulsed with mutated peptide AVCPWTWLR (SEQ ID NO: 141). Effector cells treated with PMA served as a positive control. Effector cells cultured alone (without target cells) served as a negative control. Untransduced PBL co-cultured with target cells served as a negative control.

FIGS. 3A-3C are graphs showing the percentage of effector cells (positive for expression of mTCR and CD8) that upregulated 4-1BB expression following co-culture with target cells. The effector cells were healthy donor PBL independently transduced with a retroviral vector encoding the 3333 TCR-A (3A), 3333 TCR-C(3B), or 1913 TCR-41BB (3C). The target cells were HLA-A*03:01+. HLA-A*11:01+DCs that were (i) transfected with P16INK4a RNA encoding mutated peptide AVCPWTWLR (SEQ ID NO: 141) at the amounts shown (circles) or (ii) transfected with P16INK4a RNA encoding the corresponding WT peptide at the amounts shown (squares).

FIGS. 3D-3F are graphs showing the percentage of effector cells (positive for expression of mTCR and CD8) that upregulated 4-1BB expression following co-culture with target cells. The effector cells were healthy donor PBL independently transduced with a retroviral vector encoding the 3333 TCR-A (3D), 3333 TCR-C(3E), or 1913 TCR-41BB (3F). The target cells were HLA-A*03:01+. HLA-A*11:01+DCs that were (i) pulsed with mutated peptide AVCPWTWLR (SEQ ID NO: 141) at the concentrations shown (circles) or (ii) pulsed with an irrelevant CDKN2A peptide at the concentrations shown (squares).

FIG. 4 is a graph showing the percentage of effector cells (positive for expression of mTCR and CD8) that upregulated 4-1BB expression following co-culture with target cells. The effector cells were healthy donor PBL independently transduced with a retroviral vector encoding 3333 TCR-A, 3333 TCR-C, or 1913 TCR-41BB. Target cells were (i) autologous tumor from Patient 3333, (ii) autologous tumor from Patient 3333 pulsed with CDKN24 frameshift mutated peptide AVCPWTWLR (SEQ ID NO: 141), (iii) autologous tumor from Patient 1913, (iv) autologous tumor from Patient 1913 pulsed with CDKN2A frameshift mutated peptide AVCPWTWLR (SEQ ID NO: 141), or (v) autologous tumor from Patient 2514. The target cells that were positive for expression of mutated peptide AVCPWTWLR (SEQ ID NO: 141), HLA-A*03:01, or HLA-A*11:01 are indicated by the symbol “+.” The target cells that were negative for expression of HLA-A*03:01 or HLA-A*11:01 are indicated by the symbol “−.”

FIG. 5 shows graphs depicting experimental data (dot plots) illustrating the percentage of the CD8+CD45RO+CD45RA−HLADR+CD39+CD103+anti-tumor T cell fraction of a peripheral blood sample from Patient 4286 which bound to 9-mer LLVDLAEEL (SEQ ID NO: 142) tetramer (middle plot: TET+0.062) or 10-mer RLLVDLAEEL (SEQ ID NO: 143) tetramer (right plot: TET+0).10) or to no tetramer (left plot: TET+ ( ).

FIG. 6 shows graphs depicting experimental data (dot plots) illustrating the percentage of PBL independently transduced with 4286 TCR 10-1, 4286 TCR 10-2, 4286 TCR 10-3, or 4286 TCR 10-4 which bound to 9-mer LLVDLAEEL (SEQ ID NO: 142) tetramer or 10-mer RLLVDLAEEL (SEQ ID NO: 143) tetramer or to no tetramer (negative control).

FIGS. 7A-7H are graphs showing the percentage of effector cells (positive for expression of the murine TCR constant region and CD8) that upregulated 4-1BB expression following co-culture with target cells. The effector cells were healthy donor PBL independently transduced with a retroviral vector encoding 4286 TCR 10-1 (7A and 7E), 4286 TCR 10-2 (7B and 7F), 4286 TCR 10-3 (7C and 7G), or 4286 TCR 10-4 (7D and 7H). The target cells were HLA-A*02:01+DCs that were pulsed with (i) mutated 9-mer peptide LLVDLAEEL (SEQ ID NO: 142) (7A, 7B, 7C, and 7D) at the concentrations shown (circles), (ii) corresponding WT 9-mer peptide (7A, 7B, 7C, and 7D) at the concentrations shown (squares). (iii) mutated 10-mer peptide RLLVDLAEEL (SEQ ID NO: 143) (7E, 7F, 7G, and 7H) at the concentrations shown (circles), or (iv) the corresponding WT 10-mer peptide (7E, 7F, 7G, and 7H) at the concentrations shown (squares).

### DETAILED DESCRIPTION OF THE INVENTION

CDKN2A is a gene located at band p21.3 of chromosome 9 in humans. CDKN2A encodes two distinct tumor suppressor proteins translated from alternatively spliced mRNAs. The alpha RNA transcript of CDKN2A comprises exons 1α, 2 and 3 and encodes the cyclin-dependent kinase inhibitor p16INK4a. Wild-type (WT) p16INK4a has the amino acid sequence of SEQ ID NO: 147. The beta RNA transcript of CDKN2A comprises exons 1B, 2 and 3 and encodes p14ARF. WT p14ARF has the amino acid sequence of SEQ ID NO: 148. The primary amino acid sequences of p14ARF and p16INK4a are completely unrelated because they are produced by translating the common exon 2 sequences in different reading frames.

CDKN2A is the second most commonly inactivated gene through somatic mutations across human cancers. CDKN2A mutations have a high prevalence in melanoma (about 50%), as well as in gastrointestinal and pancreatic cancers. Germline mutations in CDKN2A may also predispose humans to develop a variety of cancers. Somatic mutations arising in cancer cells that lead to inactivation of the p16INK4a and p14ARF proteins are found in about 10% of melanoma and pancreatic cancers.

Within the CDKN2A gene, about 51% of mutations are frameshift mutations. CDKN2A frameshift mutations are common in melanoma. The neoepitope AVCPWTWLR (SEQ ID NO: 141) is generated by any frameshift mutation in the nucleotide sequence encoding the amino acid residues positioned (i) between the methionine at position 54 (M54) and the tryptophan at position 100 (W100) of the of the p16INK4a protein (−1 or +2 indels) or (ii) between the glycine at position 69 (G69) and the leucine at position 124 (L124) of the p14ARF protein (−2 or +1 indels). A frame shift is described using “fs” after the first amino acid residue affected by the change. Examples of full-length mutated amino acid sequences encoded by CDKN2A with a frameshift mutation include SEQ ID NO: 150 (encoded by CDKN2A with frameshift mutation c.304delG, p.A102fs). SEQ ID NO: 151 (encoded by CDKN2A with frameshift mutation c.304delG, p.A102fs). SEQ ID NO: 152 (encoded by CDKN2A with frameshift mutation c.222_223delCG, c.G220T), and SEQ ID NO: 153 (encoded by CDKN2A with frameshift mutation c.222_223delCG, c.G220T). The mutated amino acid sequences of SEQ ID NOs: 151 and 152 each include the neoepitope AVCPWTWLR (SEQ ID NO: 141).

About 2% of CDKN2A mutations are nonsynonymous mutations in the nucleotide sequence encoding the amino acid residue at position 114, resulting in a substitution of proline with leucine (referred to as “P114L”) (a nonsynonymous substitution). An example of a full-length p16INK4a amino acid sequence with the P114L mutation is SEQ ID NO: 149.

An aspect of the invention provides an isolated or purified TCR having antigenic specificity for a mutated amino acid sequence encoded by mutated CDKN2A. In aspects of the invention, the mutated CDKN2A is mutated human CDKN2A. Hereinafter, references to a “TCR” also refer to functional portions and functional variants of the TCR, unless specified otherwise.

The mutated amino acid sequence may be encoded by CDKN2A with a frameshift mutation, as described above. In an aspect of the invention, the TCR has antigenic specificity for the mutated amino acid sequence of AVCPWTWLR (SEQ ID NO: 141). In this regard, aspects of the invention provide TCRs with antigenic specificity for any mutated human p16INK4a or mutated human p14ARF protein, polypeptide or peptide comprising the amino acid sequence of AVCPWTWLR (SEQ ID NO: 141).

The mutated amino acid sequence may be encoded by CDKN2A with a nonsynonymous substitution, as described above. In an aspect of the invention, the mutated amino acid sequence comprises a human p16INK4a amino acid sequence with a substitution of proline at position 114 with leucine, wherein position 114 is defined by reference to the corresponding WT p16INK4a protein (SEQ ID NO: 147). In this regard, aspects of the invention provide TCRs with antigenic specificity for any human p16INK4a protein. polypeptide or peptide amino acid sequence with a P114L mutation.

Mutations and substitutions of p16INK4a are defined herein by reference to the amino acid sequence of the corresponding WT p16INK4a protein. Thus, mutations and substitutions of p16INK4a are described herein by reference to the amino acid residue present at a particular position in WT p16INK4a protein (namely, position 114), followed by the position number, followed by the amino acid residue with which that residue has been replaced in the particular mutation or substitution under discussion. A p16INK4a amino acid sequence (e.g., a p16INK4a peptide) may comprise fewer than all of the amino acid residues of the full-length. WT p16INK4a protein. Accordingly, position 114 is defined herein by reference to the WT full-length p16INK4a protein (namely, SEQ ID NO: 147) with the understanding that the actual position of the corresponding residue in a particular example of a p16INK4a amino acid sequence may be different. When the positions are as defined by SEQ ID NO: 147, the term “P114” refers to the proline normally present at position 114 of SEQ ID NO: 147, and “P114L” indicates that the proline normally present at position 114 of any one of SEQ ID NO: 147 is replaced byleucine. For example, when a particular example of a p16INK4a amino acid sequence is e.g., RLPVDLAEEL (SEQ ID NO: 145) (an exemplary WT p16INK4a peptide corresponding to contiguous amino acid residues 112 to 121 of SEQ ID NO: 147), “P114L” refers to a substitution of the underlined proline in SEQ ID NO: 145 with leucine, even though the actual position of the underlined glycine in SEQ ID NO: 145 is 3. Human p16INK4a amino acid sequences with the P114L mutation are hereinafter referred to as “P114L p16INK4a.”

Hereinafter, references to “mutated (DKN2A peptide” collectively refer to the mutated amino acid sequences encoded by mutated CDKN2A described herein (having the frameshift mutation described herein or the nonsynonymous substitution described herein), unless specified otherwise.

In an aspect of the invention, the TCR has antigenic specificity for a mutated CDKN2A peptide described above, wherein the mutated CDKN2A peptide has anylength. In an aspect of the invention, the mutated CDKN2A peptide has anylength suitable for binding to any of the HLA Class I molecules described herein. For example, the TCR may have antigenic specificity for a mutated CDKN2A peptide, the mutated CDKN2A peptide having a length of about 9 to about 10 amino acid residues. In an aspect of the invention, the P114L p16INK4a peptide may comprise any contiguous amino acid residues of mutated p16INK4a protein which include the P114L mutation. In an aspect of the invention, the mutated CDKN2A peptide may comprise any contiguous amino acid residues of mutated p16INK4a protein or mutated p14ARF protein comprising AVCPWTWLR (SEQ ID NO: 141) and which is encoded by the CDKN2A frameshift mutation described herein. In an aspect of the invention, the TCR may have antigenic specificity for a mutated CDKN2A peptide, the mutated CDKN2A peptide having a length of about 9 amino acid residues or about 10 amino acid residues. Examples of specific peptides, which may be recognized by the inventive TCR are 9-mer AVCPWTWLR (SEQ ID NO: 141), 9-mer LLVDLAEEL (SEQ ID NO: 142), or 10-mer RLLVDLAEEL (SEQ ID NO: 143). In an aspect of the invention, the TCR does not have antigenic specificity for the wild-type human p16INK4a amino acid sequence of wild-type amino acid sequence of LPVDLAEEL (SEQ ID NO: 144) or RLPVDLAEEL (SEQ ID NO: 145).

In an aspect of the invention, the inventive TCRs are able to recognize the mutated CDKN2A peptide presented by an HLA Class I molecule. In this regard, the TCR may elicit an immune response upon binding to the mutated CDKN2A peptide within the context of an HLA Class I molecule. The inventive TCRs are able to recognize mutated CDKN2A peptide that is presented by an HLA Class I molecule and may bind to the HLA Class I molecule in addition to the mutated CDKN2A peptide.

In an aspect of the invention, the HLA Class I molecule is an HLA-A molecule. The HLA-A molecule is a heterodimer of an a chain and B2 microglobulin. The HLA-A a chain may be encoded by an HLA-A gene. B2 microglobulin binds non-covalently to the a1, a2 and a3 domains of the a chain to build the HLA-A complex. The HLA-A molecule may be any HLA-A molecule.

In an aspect of the invention, the HLA Class I molecule is an HLA-All molecule. The HLA-All molecule may be any HLA-All molecule. Examples of HLA-All molecules may include, but are not limited to, those encoded by the HLA-A*11:01. HLA-A*11:02. HLA-A*11:03, or HLA-A*11:04 alleles. In an aspect, the HLA Class I molecule is encoded by the HLA-A*11:01 allele.

In an aspect of the invention, the HLA Class I molecule is an HLA-A3 molecule. The HLA-A3 molecule may be any HLA-A3 molecule. Examples of HLA-A3 molecules may include, but are not limited to, those encoded by the HLA-A*3:01, HLA-A*3:02, or HLA-A*3:05 alleles. In an aspect, the HLA Class I molecule is encoded by the HLA-A*3:01 allele.

In an aspect of the invention, the HLA Class I molecule is an HLA-A2 molecule. The HLA-A2 molecule may be any HLA-A2 molecule. Examples of HLA-A2 molecules may include, but are not limited to, those encoded by the HLA-A*2:01, HLA-A*2:02, HLA-A*2:03, HLA-A*2:05, HLA-A*2:06, HLA-A*2:07, or HLA-A*2:11 alleles. In an aspect, the HLA Class I molecule is encoded by the HLA-A*2:01 allele.

The TCRs of the invention may provide any one or more of a variety of advantages, including when expressed by cells used for adoptive cell transfer. The CDKN2A mutations described herein are expressed by cancer cells and are not expressed by normal, noncancerous cells. Without being bound to a particular theory or mechanism, it is believed that the inventive TCRs advantageously target the destruction of cancer cells while minimizing or eliminating the destruction of normal, non-cancerous cells, thereby reducing, for example, by minimizing or eliminating, toxicity. The inventive TCRs may, advantageously, successfully treat or prevent cancers expressing the CDKN2A mutations described herein that do not respond to other types of treatment such as, for example, chemotherapy, surgery, or radiation. There are currently no targeted drugs against CDKN2A mutations. Additionally, the inventive TCRs may provide highly avid recognition of the mutated CDKN2A peptides described herein, which may provide the ability to recognize unmanipulated tumor cells (e.g., tumor cells that have not been treated with interferon (IFN)-γ, transfected with a vector encoding one or both of mutated CDKN2A peptide and and any of the HLA-A molecules described herein, pulsed with a mutated CDKN2.4 peptide, or a combination thereof). Moreover, the HLA-A*03:01, HLA-A*11:01, and HLA-A*02:01 alleles together account for about 8%, about 7%, and about 40%, respectively, within the population in the United States. Accordingly, the inventive TCRs may increase the number of immunotherapy-eligible cancer patients to include those patients that express the HLA-A*03:01. HLA-A*11:01, or HLA-A*02:01 allele who may not be eligible for immunotherapy using TCRs that recognize mutated CDKN2A peptide presented by other MHC molecules. Moreover, the inventive TCRs, polypeptides and proteins comprise human CDR and variable region amino acid sequences, which may reduce the risk of rejection by the human immune system as compared to, e.g., TCRs, polypeptides and proteins comprising mouse CDR and variable region amino acid sequences. Moreover, autologous tumor-specific T cells from cancer patients can target tumor mutations, but are often terminally exhausted in their phenotypic states. The inventive isolated TCRs can be used to engineer a patient's own T cells (e.g., blood-derived naïve T cells) to target their tumors.

The phrase “antigenic specificity,” as used herein, means that the TCR can specifically bind to and immunologically recognize mutated CDKN2.4 peptide with an avidity that is higher relative to that of the wildtype nonmutated CDKN2A counterpart. For example, a TCR may be considered to have “antigenic specificity” for mutated CDKN2A peptide if about 1×10⁴ to about 1×10⁵ T cells expressing the TCR secrete at least about 200 μg/mL or more (e.g., 200 μg/mL or more, 300 μg/mL or more, 400 μg/mL or more, 500 μg/mL, or more, 600 μg/mL or more, 700 μg/mL or more, 1000 μg/mL or more, 5,000 μg/mL, or more, 7.000 μg/mL or more, 10.000 μg/mL or more, 20,000 μg/mL or more, or a range defined by any two of the foregoing values) of IFN-γ upon co-culture with (a) antigen-negative, HLA Class I molecule positive target cells pulsed with a low concentration of mutated CDKN2A peptide (e.g., about 0.05 ng/mL to about 10 ng/ml, 1 ng/mL, 2 ng/ml, 5 ng/ml, 8 ng/mL, 10 ng/ml, or a range defined by any two of the foregoing values) or (b) antigen-negative, HLA Class I molecule positive target cells into which a nucleotide sequence encoding mutated CDKN2A peptide has been introduced such that the target cell expresses mutated CDKN2A peptide. Cells expressing the inventive TCRs may also secrete IFN-γ upon co-culture with antigen-negative. HLA Class I molecule positive target cells pulsed with higher concentrations of mutated CDKN2A peptide or upon culture with tumor cells expressing the mutated CDKN2A peptide and HLA Class I molecule. The HLA Class I molecule may be any of the HLA Class I molecules described herein.

Alternatively or additionally, a TCR may be considered to have “antigenic specificity” for mutated CDKN2A peptide if T cells expressing the TCR secrete at least twice (e.g., five times) as much IFN-γ upon co-culture with (a) antigen-negative, HLA Class I molecule positive target cells pulsed with a low concentration of mutated CDKN2A peptide or (b) antigen-negative. HLA Class I molecule positive target cells into which a nucleotide sequence encoding mutated CDKN2A peptide has been introduced such that the target cell expresses mutated CDKN2A peptide as compared to the amount of IFN-γ expressed by a negative control. The negative control may be, for example, (i) T cells expressing the TCR, co-cultured with (a) antigen-negative, HLA Class I molecule positive target cells pulsed with the same concentration of an irrelevant peptide (e.g., some other peptide with a different sequence from the mutated CDKN2A peptide) or (b) antigen-negative, HLA Class I molecule positive target cells into which a nucleotide sequence encoding an irrelevant peptide has been introduced such that the target cell expresses the irrelevant peptide, or (ii) untransduced T cells (e.g., derived from PBMC, which do not express the TCR) co-cultured with (a) antigen-negative, HLA Class I molecule positive target cells pulsed with the same concentration of mutated CDKN2A peptide or (b) antigen-negative. HLA Class I molecule positive target cells into which a nucleotide sequence encoding mutated CDKN2A peptide has been introduced such that the target cell expresses mutated CDKN2A peptide. The HLA Class I molecule expressed by the target cells of the negative control would be the same HLA Class I molecule expressed by the target cells that are co-cultured with the T cells being tested. Target cells may include, for example, tumor cells (human or mouse-derived), antigen presenting cell lines, or cell lines that express the mutated CDKN2A peptide and appropriate HLA molecule. The HLA Class I molecule may be any of the HLA Class I molecules described herein. IFN-γ secretion may be measured by methods known in the art such as, for example, enzyme-linked immunosorbent assay (ELISA).

Alternatively or additionally, a TCR may be considered to have “antigenic specificity” for mutated CDKN2A peptide if at least twice (e.g., five times) as many of the numbers of T cells expressing the TCR secrete IFN-γ upon co-culture with (a) antigen-negative, HLA Class I molecule positive target cells pulsed with a low concentration of mutated CDKN2.4 peptide or (b) antigen-negative. HLA Class I molecule positive target cells into which a nucleotide sequence encoding mutated CDKN2A peptide has been introduced such that the target cell expresses mutated CDKN24 peptide as compared to the numbers of negative control T cells that secrete IFN-γ. The HLA Class I molecule, concentration of peptide, and the negative control may be as described herein with respect to other aspects of the invention. The numbers of cells secreting IFN-γ may be measured by methods known in the art such as, for example, ELISPOT.

Alternatively or additionally, a TCR may be considered to have “antigenic specificity” for mutated CDKN24 peptide if T cells expressing the TCR upregulate expression of one or more T-cell activation markers as measured by, for example, flow cytometry after stimulation with target cells expressing mutated CDKN24 peptide. Examples of T-cell activation markers include 4-1BB, OX40, CD107a, CD69, and cytokines that are upregulated upon antigen stimulation (e.g., tumor necrosis factor (TNF), interleukin (IL)-2, etc.).

An aspect of the invention provides a TCR comprising two polypeptides (i.e., polypeptide chains), such as an alpha (a) chain of a TCR, a beta (B) chain of a TCR, a gamma (Y) chain of a TCR, a delta (8) chain of a TCR, or a combination thereof. The polypeptides of the inventive TCR can comprise any amino acid sequence, provided that the TCR has antigenic specificity for mutated CDKN2A peptide. In some aspects, the TCR is non-naturally occurring.

In an aspect of the invention, the TCR comprises two polypeptide chains, each of which comprises a variable region comprising a complementarity determining region (CDR) 1, a CDR2, and a CDR3 of a TCR. In an aspect of the invention, the TCR comprises a first polypeptide chain comprising a CDR1 comprising the amino acid sequence of SEQ ID NO: 1 (CDR1 of α chain of 3333 TCR-A), a CDR2 comprising the amino acid sequence of SEQ ID NO: 2 (CDR2 of α chain of 3333 TCR-A), and a CDR3 comprising the amino acid sequence of SEQ ID NO: 3 (CDR3 of α chain of 3333 TCR-A), and a second polypeptide chain comprising a CDR1 comprising the amino acid sequence of SEQ ID NO: 4 (CDR1 of β chain of 3333 TCR-A), a CDR2 comprising the amino acid sequence of SEQ ID NO: 5 (CDR2 of β chain of 3333 TCR-A), and a CDR3 comprising the amino acid sequence of SEQ ID NO: 6 (CDR3 of β chain of 3333 TCR-A).

In another aspect of the invention, the TCR comprises a first polypeptide chain comprising a CDR1 comprising the amino acid sequence of SEQ ID NO: 11 (CDR1 of a chain of 3333 TCR-C), a CDR2 comprising the amino acid sequence of SEQ ID NO: 12 (CDR2 of α chain of 3333 TCR-C), and a CDR3 comprising the amino acid sequence of SEQ ID NO: 13 (CDR3 of α chain of 3333 TCR-C), and a second polypeptide chain comprising a CDR1 comprising the amino acid sequence of SEQ ID NO: 14 (CDR1 of β chain of 3333 TCR-C), a CDR2 comprising the amino acid sequence of SEQ ID NO: 15 (CDR2 of β chain of 3333 TCR-C), and a CDR3 comprising the amino acid sequence of SEQ ID NO: 16 (CDR3 of β chain of 3333 TCR-C).

In another aspect of the invention, the TCR comprises a first polypeptide chain comprising a CDR1 comprising the amino acid sequence of SEQ ID NO: 21 (CDR1 of a chain of 1913 TCR-41BB), a CDR2 comprising the amino acid sequence of SEQ ID NO: 22 (CDR2 of α chain of 1913 TCR-41BB), and a CDR3 comprising the amino acid sequence of SEQ ID NO: 23 (CDR3 of α chain of 1913 TCR-41BB), and a second polypeptide chain comprising a CDR1 comprising the amino acid sequence of SEQ ID NO: 24 (CDR1 of β chain of 1913 TCR-41BB), a CDR2 comprising the amino acid sequence of SEQ ID NO: 25 (CDR2 of β chain of 1913 TCR-41BB), and a CDR3 comprising the amino acid sequence of SEQ ID NO: 26 (CDR3 of β chain of 1913 TCR-41BB).

In another aspect of the invention, the TCR comprises a first polypeptide chain comprising a CDR1 comprising the amino acid sequence of SEQ ID NO: 31 (CDR1 of a chain of 4286 TCR 10-1), a CDR2 comprising the amino acid sequence of SEQ ID NO: 32 (CDR2 of α chain of 4286 TCR 10-1), and a CDR3 comprising the amino acid sequence of SEQ ID NO: 33 (CDR3 of α chain of 4286 TCR 10-1), and a second polypeptide chain comprising a CDR1 comprising the amino acid sequence of SEQ ID NO: 34 (CDR1 of β chain of 4286 TCR 10-1), a CDR2 comprising the amino acid sequence of SEQ ID NO: 35 (CDR2 of β chain of 4286 TCR 10-1), and a CDR3 comprising the amino acid sequence of SEQ ID NO: 36 (CDR3 of β chain of 4286 TCR 10-1).

In another aspect of the invention, the TCR comprises a first polypeptide chain comprising a CDR1 comprising the amino acid sequence of SEQ ID NO: 41 (CDR1 of a chain of 4286 TCR 10-2), a CDR2 comprising the amino acid sequence of SEQ ID NO: 42 (CDR2 of α chain of 4286 TCR 10-2), and a CDR3 comprising the amino acid sequence of SEQ ID NO: 43 (CDR3 of α chain of 4286 TCR 10-2), and a second polypeptide chain comprising a CDR1 comprising the amino acid sequence of SEQ ID NO: 44 (CDR1 of β chain of 4286 TCR 10-2), a CDR2 comprising the amino acid sequence of SEQ ID NO: 45 (CDR2 of β chain of 4286 TCR 10-2), and a CDR3 comprising the amino acid sequence of SEQ ID NO: 46 (CDR3 of β chain of 4286 TCR 10-2).

In another aspect of the invention, the TCR comprises a first polypeptide chain comprising a CDR1 comprising the amino acid sequence of SEQ ID NO: 51 (CDR1 of a chain of 4286 TCR 10-3), a CDR2 comprising the amino acid sequence of SEQ ID NO: 52 (CDR2 of α chain of 4286 TCR 10-3), and a CDR3 comprising the amino acid sequence of SEQ ID NO: 53 (CDR3 of α chain of 4286 TCR 10-3), and a second polypeptide chain comprising a CDR1 comprising the amino acid sequence of SEQ ID NO: 54 (CDR1 of β chain of 4286 TCR 10-3), a CDR2 comprising the amino acid sequence of SEQ ID NO: 55 (CDR2 of β chain of 4286 TCR 10-3), and a CDR3 comprising the amino acid sequence of SEQ ID NO: 56 (CDR3 of β chain of 4286 TCR 10-3).

In another aspect of the invention, the TCR comprises a first polypeptide chain comprising a CDR1 comprising the amino acid sequence of SEQ ID NO: 61 (CDR1 of a chain of 4286 TCR 10-4), a CDR2 comprising the amino acid sequence of SEQ ID NO: 62 (CDR2 of α chain of 4286 TCR 10-4), and a CDR3 comprising the amino acid sequence of SEQ ID NO: 63 (CDR3 of α chain of 4286 TCR 10-4), and a second polypeptide chain comprising a CDR1 comprising the amino acid sequence of SEQ ID NO: 64 (CDR1 of β chain of 4286 TCR 10-4), a CDR2 comprising the amino acid sequence of SEQ ID NO: 65 (CDR2 of β chain of 4286 TCR 10-4), and a CDR3 comprising the amino acid sequence of SEQ ID NO: 66 (CDR3 of β chain of 4286 TCR 10-4).

In this regard, the inventive TCR can comprise any one or more of the amino acid sequences selected from the group consisting of SEQ ID NOs: 1-6, 11-16, 21-26, 31-36, 41-46, 51-56, and 61-66. In an aspect of the invention, the TCR comprises the amino acid sequences of: (a) all of SEQ ID NOs: 1-3: (b) all of SEQ ID NOs: 4-6: (c) all of SEQ ID NOs: 1-6: (d) all of SEQ ID NOs: 11-13: (e) all of SEQ ID NOs: 14-16; (f) all of SEQ ID NOs: 11-16: (g) all of SEQ ID NOs: 21-23: (h) all of SEQ ID NOs: 24-26: (i) all of SEQ ID NOs: 21-26: (i) all of SEQ ID NO: 31-33: (k) all of SEQ ID NO: 34-36: (1) all of SEQ ID NO: 31-36: (m) all of SEQ ID NO: 41-43: (n) all of SEQ ID NO: 44-46: (0) all of SEQ ID NO: 41-46: (p) all of SEQ ID NO: 51-53; (q) all of SEQ ID NO: 54-56; (r) all of SEQ ID NO: 51-56: (s) all of SEQ ID NO: 61-63: (t) all of SEQ ID NO: 64-66; or (u) all of SEQ ID NO: 61-66. In an especially preferred aspect, the TCR comprises the amino acid sequences of: (a) all of SEQ ID NOs: 1-6. (b) all of SEQ ID NOs: 11-16. (c) all of SEQ ID NOs: 21-26, (d) all of SEQ ID NOs: 31-36, (e) all of SEQ ID NOs: 41-46, (f) all of SEQ ID NOs: 51-56, or (g) all of SEQ ID NOs: 61-66.

In an aspect of the invention, the TCR comprises an amino acid sequence of a variable region of a TCR comprising the CDRs set forth above. In this regard, the TCR can comprise the amino acid sequence of: (1) SEQ ID NO: 7 (predicted sequence of variable region of α chain of 3333 TCR-A without N-terminal signal peptide): (2) SEQ ID NO: 8 (predicted sequence of variable region of β chain of 3333 TCR-A without N-terminal signal peptide): (3) SEQ ID NO: 9 (variable region of α chain of 3333 TCR-A with N-terminal signal peptide): (4) SEQ ID NO: 10 (variable region of β chain of 3333 TCR-A with N-terminal signal peptide): (5) SEQ ID NO: 17 (predicted sequence of variable region of a chain of 3333 TCR-C without N-terminal signal peptide): (6) SEQ ID NO: 18 (predicted sequence of variable region of β chain of 3333 TCR-C without N-terminal signal peptide): (7) SEQ ID NO: 19 (variable region of α chain of 3333 TCR-C with N-terminal signal peptide): (8) SEQ ID NO: 20 (variable region of β chain of 3333 TCR-C with N-terminal signal peptide): (9) SEQ ID NO: 27 (predicted sequence of variable region of α chain of 1913 TCR-41BB without N-terminal signal peptide): (10) SEQ ID NO: 28 (predicted sequence of variable region of β chain of 1913 TCR-41BB without N-terminal signal peptide): (11) SEQ ID NO: 29 (variable region of α chain of 1913 TCR-41BB with N-terminal signal peptide): (12) SEQ ID NO: 30 (variable region of β chain of 1913 TCR-41BB with N-terminal signal peptide): (13) SEQ ID NO: 37 (predicted sequence of variable region of α chain of 4286 TCR 10-1 without N-terminal signal peptide): (14) SEQ ID NO: 38 (predicted sequence of variable region of β chain of 4286 TCR 10-1 without N-terminal signal peptide): (15) SEQ ID NO: 39 (variable region of α chain of 4286 TCR 10-1 with N-terminal signal peptide): (16) SEQ ID NO: 40 (variable region of β chain of 4286 TCR 10-1 with N-terminal signal peptide): (17) SEQ ID NO: 47 (predicted sequence of variable region of α chain of 4286 TCR 10-2 without N-terminal signal peptide): (18) SEQ ID NO: 48 (predicted sequence of variable region of β chain of 4286 TCR 10-2 without N-terminal signal peptide): (19) SEQ ID NO: 49 (variable region of α chain of 4286 TCR 10-2 with N-terminal signal peptide): (20) SEQ ID NO: 50 (variable region of β chain of 4286 TCR 10-2 with N-terminal signal peptide): (21) SEQ ID NO: 57 (predicted sequence of variable region of α chain of 4286 TCR 10-3 without N-terminal signal peptide): (22) SEQ ID NO: 58 (predicted sequence of variable region of β chain of 4286 TCR 10-3 without N-terminal signal peptide): (23) SEQ ID NO: 59 (variable region of α chain of 4286 TCR 10-3 with N-terminal signal peptide): (24) SEQ ID NO: 60 (variable region of β chain of 4286 TCR 10-3 with N-terminal signal peptide): (25) SEQ ID NO: 67 (predicted sequence of variable region of α chain of 4286 TCR 10-4 without N-terminal signal peptide): (26) SEQ ID NO: 68 (predicted sequence of variable region of B chain of 4286 TCR 10-4 without N-terminal signal peptide): (27) SEQ ID NO: 69 (variable region of α chain of 4286 TCR 10-4 with N-terminal signal peptide): (28) SEQ ID NO: 70 (variable region of β chain of 4286 TCR 10-4 with N-terminal signal peptide): (29) both of SEQ ID NOs: 7 and 8: (30) both of SEQ ID NOs: 9 and 10: (31) both of SEQ ID NOs: 17 and 18: (32) both of SEQ ID NOs: 19 and 20; (33) both of SEQ ID NOs: 27 and 28: (34) both of SEQ ID NOs: 29 and 30: (35) both of SEQ ID NOs: 37 and 38: (36) both of SEQ ID NOs: 39 and 40; (37) both of SEQ ID NOs: 47 and 48: (38) both of SEQ ID NOs: 49 and 50; (39) both of SEQ ID NOs: 57 and 58: (40) both of SEQ ID NOs: 59 and 60: (41) both of SEQ ID NOs: 67 and 68; or (42) both of SEQ ID NOs: 69 and 70.

Preferably, the TCR comprises the amino acid sequences of (i) both of SEQ ID NOs: 7 and 8, (ii) both of SEQ ID NOs: 9 and 10, (iii) both of SEQ ID NOs: 17 and 18, (iv) both of SEQ ID NOs: 19 and 20, (v) both of SEQ ID NOs: 27 and 28. (vi) both of SEQ ID NOs: 29 and 30, (vii) both of SEQ ID NOs: 37 and 38, (viii) both of SEQ ID NOs: 39 and 40, (ix) both of SEQ ID NOs: 47 and 48, (x) both of SEQ ID NOs: 49 and 50, (xi) both of SEQ ID NOs: 57 and 58. (xii) both of SEQ ID NOs: 59 and 60. (xiii) both of SEQ ID NOs: 67 and 68, or (xiv) both of SEQ ID NOs: 69 and 70.

The inventive TCRs may further comprise an a chain constant region and a β chain constant region. The constant region may be derived from any suitable species such as, e.g., human or mouse. In an aspect of the invention, the TCRs further comprise murine α and β chain constant regions or human α and β chain constant regions. As used herein, the term “murine” or “human,” when referring to a TCR or any component of a TCR described herein (e.g., CDR, variable region, constant region, a chain, and/or β chain), means a TCR (or component thereof) which is derived from a mouse or a human, respectively, i.e., a TCR (or component thereof) that originated from or was, at one time, expressed by a mouse T cell or a human T cell, respectively.

An aspect of the invention provides a chimeric TCR comprising a human variable region and a murine constant region, wherein the TCR has antigenic specificity for a mutated amino acid sequence encoded by mutated CDKN2A. The murine constant region may provide any one or more advantages. For example, the murine constant region may diminish mispairing of the inventive TCR with the endogenous TCRs of the host cell into which the inventive TCR is introduced. Alternatively or additionally, the murine constant region may increase expression of the inventive TCR as compared to the same TCR with a human constant region. The chimeric TCR may comprise the amino acid sequence of SEQ ID NO: 75 (WT murine α chain constant region). SEQ ID NO: 76 (WT murine β chain constant region), or both SEQ ID NOs: 75 and 76. Preferably, the inventive TCR comprises the amino acid sequences of both of SEQ ID NOs: 75 and 76. The chimeric TCR may comprise any of the murine constant regions described herein in combination with any of the CDR regions as described herein with respect to other aspects of the invention. In this regard, the TCR may comprise the amino acid sequences of: (a) all of SEQ ID NOs: 1-3 and 75, (b) all of SEQ ID NOs: 4-6 and 76, (c) all of SEQ ID NOs: 1-6 and 75-76, (d) all of SEQ ID NOs: 11-13 and 75, (e) all of SEQ ID NOs: 14-16 and 76, (f) all of SEQ ID NOs: 11-16 and 75-76, (g) all of SEQ ID NOs: 21-23 and 75. (h) all of SEQ ID NOs: 24-26 and 76. (i) all of SEQ ID NOs: 21-26 and 75-76, (j) all of SEQ ID NOs: 31-33 and 75. (k) all of SEQ ID NOs: 34-36 and 76, (1) all of SEQ ID NOs: 31-36 and 75-76, (m) all of SEQ ID NOs: 41-43 and 75, (n) all of SEQ ID NOs: 44-46 and 76, (o) all of SEQ ID NOs: 41-46 and 75-76, (p) all of SEQ ID NOs: 51-53 and 75. (q) all of SEQ ID NOs: 54-56 and 76. (r) all of SEQ ID NOs: 51-56 and 75-76. (s) all of SEQ ID NOs: 61-63 and 75. (t) all of SEQ ID NOs: 64-66 and 76, or (u) all of SEQ ID NOs: 61-66 and 75-76.

In another aspect of the invention, the chimeric TCR may comprise any of the murine constant regions described herein in combination with any of the variable regions described herein with respect to other aspects of the invention. In this regard, the TCR may comprise the amino acid sequences of: (1) both of SEQ ID NOs: 7 and 75, (2) both of SEQ ID NOs: 8 and 76, (3) both of SEQ ID NOs: 9 and 75. (4) both of SEQ ID NOs: 10 and 76, (5) both of SEQ ID NOs: 17 and 75, (6) both of SEQ ID NOs: 18 and 76. (7) both of SEQ ID NOs: 19 and 75. (8) both of SEQ ID NOs: 20 and 76. (9) both of SEQ ID NOs: 27 and 75, (10) both of SEQ ID NOs: 28 and 76, (11) both of SEQ ID NOs: 29 and 75. (12) both of SEQ ID NOs: 30 and 76, (13) both of SEQ ID NOs: 37 and 75. (14) both of SEQ ID NOs: 38 and 76, (15) both of SEQ ID NOs: 39 and 75, (16) both of SEQ ID NOs: 40 and 76. (17) both of SEQ ID NOs: 47 and 75, (18) both of SEQ ID NOs: 48 and 76, (19) both of SEQ ID NOs: 49 and 75, (20) both of SEQ ID NOs: 50 and 76, (21) both of SEQ ID NOs: 57 and 75, (22) both of SEQ ID NOs: 58 and 76, (23) both of SEQ ID NOs: 59 and 75, (24) both of SEQ ID NOS: 60 and 76, (25) both of SEQ ID NOs: 67 and 75. (26) both of SEQ ID NOs: 68 and 76, (27) both of SEQ ID NOs: 69 and 75, (28) both of SEQ ID NOs: 70 and 76, (29) all of SEQ ID NOs: 7-8 and 75-76, (30) all of SEQ ID NOs: 9-10 and 75-76, (31) all of SEQ ID NOs: 17-18 and 75-76, (32) all of SEQ ID NOs: 19-20 and 75-76, (33) all of SEQ ID NOs: 27-28 and 75-76. (34) all of SEQ ID NOs: 29-30 and 75-76. (35) all of SEQ ID NOs: 37-38 and 75-76, (36) all of SEQ ID NOs: 39-40 and 75-76, (37) all of SEQ ID NOs: 47-48 and 75-76. (38) all of SEQ ID NOs: 49-50 and 75-76, (39) all of SEQ ID NOs: 57-58 and 75-76, (40) all of SEQ ID NOs: 59-60 and 75-76, (41) all of SEQ ID NOs: 67-68 and 75-76, or (42) all of SEQ ID NOs: 69-70 and 75-76.

In an aspect of the invention, the TCR comprises a substituted constant region. In this regard, the TCR may comprise the amino acid sequence of any of the TCRs described herein with one, two, three, or four amino acid substitution(s) in the constant region of one or both of the α and β chain. Preferably, the TCR comprises a murine constant region with one, two, three, or four amino acid substitution(s) in the murine constant region of one or both of the α and β chains. In an especially preferred aspect, the TCR comprises a murine constant region with one, two, three, or four amino acid substitution(s) in the murine constant region of the a chain and one amino acid substitution in the murine constant region of the β chain. In some aspects, the TCRs comprising the substituted constant region advantageously provide one or more of increased recognition of mutated CDKN24 peptide+targets, increased expression by a host cell, diminished mispairing with endogenous TCRs, and increased anti-tumor activity as compared to the parent TCR comprising an unsubstituted (wild-type) constant region. In general, the substituted amino acid sequences of the murine constant regions of the TCR α and β chains. SEQ ID NOs: 71 and 72, respectively, correspond with all or portions of the unsubstituted murine constant region amino acid sequences SEQ ID NOs: 75 and 76, respectively, with SEQ ID NO: 71 having one, two, three, or four amino acid substitution(s) when compared to SEQ ID NO: 75 and SEQ ID NO: 72 having one amino acid substitution when compared to SEQ ID NO: 76. In this regard, an aspect of the invention provides a TCR comprising the amino acid sequences of (a) SEQ ID NO: 71 (constant region of α chain), wherein (i) X at position 48 is Thr or Cys: (ii) X at position 112 is Ser. Ala, Val, Leu, Ile, Pro, Phe. Met, or Trp: (iii) X at position 114 is Met. Ala, Val. Leu, Ile, Pro, Phe, or Trp; and (iv) X at position 115 is Gly. Ala, Val. Leu, Ile, Pro, Phe, Met, or Trp: (b) SEQ ID NO: 72 (constant region of β chain), wherein X at position 57 is Ser or Cys: or (c) both of SEQ ID NOs: 71 and 72. In an aspect of the invention, the TCR comprising SEQ ID NO: 71 does not comprise SEQ ID NO: 75 (unsubstituted murine constant region of a chain). In an aspect of the invention, the TCR comprising SEQ ID NO: 72 does not comprise SEQ ID NO: 76 (unsubstituted murine constant region of β chain).

In an aspect of the invention, the TCR comprises an α chain comprising a variable region and a constant region and a β chain comprising a variable region and a constant region. In this regard, the TCR may comprise (a) the amino acid sequence of SEQ ID NO: 77 (a chain of 3333 TCR-A with N-terminal signal peptide), wherein: (i) X at position 182 of SEQ ID NO: 77 is Thr or Cys: (ii) X at position 246 of SEQ ID NO: 77 is Ser, Ala. Val, Leu, Ile. Pro, Phe. Met, or Trp: (iii) X at position 248 of SEQ ID NO: 77 is Met. Ala, Val. Leu, Ile, Pro. Phe, or Trp; and (iv) X at position 249 of SEQ ID NO: 77 is Gly. Ala, Val. Leu, Ile, Pro. Phe, Met, or Trp: (b) the amino acid sequence of SEQ ID NO: 78 (β chain of 3333 TCR-A with N-terminal signal peptide), wherein X at position 188 of SEQ ID NO: 78 is Ser or Cys: (c) the amino acid sequences of both of SEQ ID NOs: 77 and 78: (d) the amino acid sequence of SEQ ID NO: 79 (predicted sequence of α chain of 3333 TCR-A without N-terminal signal peptide), wherein: (i) X at position 161 of SEQ ID NO: 79 is Thr or Cys: (ii) X at position 225 of SEQ ID NO: 79 is Ser, Ala. Val, Leu, Ile, Pro, Phe, Met, or Trp: (iii) X at position 227 of SEQ ID NO: 79 is Met, Ala, Val, Leu, Ile, Pro, Phe, or Trp; and (iv) X at position 228 of SEQ ID NO: 79 is Gly, Ala. Val, Leu. Ile. Pro, Phe. Met, or Trp: (e) the amino acid sequence of SEQ ID NO: 80 (predicted sequence of β chain of 3333 TCR-A without N-terminal signal peptide), wherein X at position 166 of SEQ ID NO: 80 is Ser or Cys: (f) the amino acid sequences of both of SEQ ID NOs: 79 and 80: (g) the amino acid sequence of SEQ ID NO: 81 (a chain of cysteine-substituted, LVL-modified 3333 TCR-A with N-terminal signal peptide): (h) the amino acid sequence of SEQ ID NO: 82 (β chain of cysteine-substituted. LVL-modified 3333 TCR-A with N-terminal signal peptide): (i) the amino acid sequence of SEQ ID NO: 83 (predicted sequence of α chain of cysteine-substituted, LVL-modified 3333 TCR-A without N-terminal signal peptide): (j) the amino acid sequence of SEQ ID NO: 84 (predicted sequence of β chain of cysteine-substituted. LVL-modified 3333 TCR-A without N-terminal signal peptide): (k) the amino acid sequences of both of SEQ ID NOs: 81 and 82: (l) the amino acid sequences of both of both of SEQ ID NOs: 83 and 84: (m) the amino acid sequence of SEQ ID NO: 85 (a chain of 3333 TCR-C with N-terminal signal peptide), wherein: (i) X at position 184 of SEQ ID NO: 85 is Thr or Cys: (ii) X at position 248 of SEQ ID NO: 85 is Ser, Ala, Val, Leu, Ile, Pro, Phe, Met, or Trp: (iii) X at position 250 of SEQ ID NO: 85 is Met. Ala, Val. Leu, Ile, Pro. Phe, or Trp; and (iv) X at position 251 of SEQ ID NO: 85 is Gly. Ala, Val. Leu, Ile, Pro. Phe, Met, or Trp: (n) the amino acid sequence of SEQ ID NO: 86 (β chain of 3333 TCR-C with N-terminal signal peptide), wherein X at position 188 of SEQ ID NO: 86 is Ser or Cys: (o) the amino acid sequences of both of SEQ ID NOs: 85 and 86: (p) the amino acid sequence of SEQ ID NO: 87 (predicted sequence of α chain of 3333 TCR-C without N-terminal signal peptide), wherein: (i) X at position 163 of SEQ ID NO: 87 is Thr or Cys: (ii) X at position 227 of SEQ ID NO: 87 is Ser. Ala, Val. Leu, Ile, Pro. Phe, Met, or Trp: (iii) X at position 229 of SEQ ID NO: 87 is Met, Ala, Val, Leu. Ile. Pro, Phe, or Trp; and (iv) X at position 230 of SEQ ID NO: 87 is Gly, Ala, Val, Leu, Ile, Pro, Phe, Met, or Trp; (q) the amino acid sequence of SEQ ID NO: 88 (predicted sequence of β chain of 3333 TCR-C without N-terminal signal peptide), wherein X at position 167 of SEQ ID NO: 88 is Ser or Cys; (r) the amino acid sequences of both of SEQ ID NOs: 87 and 88: (s) the amino acid sequence of SEQ ID NO: 89 (a chain of cysteine-substituted, LVL-modified 3333 TCR-C with N-terminal signal peptide): (t) the amino acid sequence of SEQ ID NO: 90 (B chain of cysteine-substituted. LVL-modified 3333 TCR-C with N-terminal signal peptide): (u) the amino acid sequence of SEQ ID NO: 91 (predicted sequence of α chain of cysteine-substituted, LVL-modified 3333 TCR-C without N-terminal signal peptide): (v) the amino acid sequence of SEQ ID NO: 92 (predicted sequence of β chain of cysteine-substituted, LVL-modified 3333 TCR-C without N-terminal signal peptide): (w) the amino acid sequences of both of SEQ ID NOs: 89 and 90: (x) the amino acid sequences of both of SEQ ID NOs: 91 and 92: (y) the amino acid sequence of SEQ ID NO: 93 (a chain of 1913 TCR-41BB with N-terminal signal peptide), wherein: (i) X at position 178 of SEQ ID NO: 93 is Thr or Cys: (ii) X at position 242 of SEQ ID NO: 93 is Ser, Ala, Val, Leu, Ile, Pro. Phe, Met, or Trp: (iii) X at position 244 of SEQ ID NO: 93 is Met. Ala, Val. Leu, Ile, Pro. Phe, or Trp; and (iv) X at position 245 of SEQ ID NO: 93 is Gly. Ala, Val. Leu, Ile, Pro, Phe, Met, or Trp: (z) the amino acid sequence of SEQ ID NO: 94 (β chain of 1913 TCR-41BB with N-terminal signal peptide), wherein X at position 190 of SEQ ID NO: 94 is Ser or Cys: (aa) the amino acid sequences of both of SEQ ID NOs: 93 and 94: (bb) the amino acid sequence of SEQ ID NO: 95 (predicted sequence of α chain of 1913 TCR-41BB without N-terminal signal peptide), wherein: (i) X at position 160 of SEQ ID NO: 95 is Thr or Cys: (ii) X at position 224 of SEQ ID NO: 95 is Ser, Ala. Val, Leu. Ile. Pro, Phe. Met, or Trp: (iii) X at position 226 of SEQ ID NO: 95 is Met, Ala, Val, Leu, Ile, Pro, Phe, or Trp; and (iv) X at position 227 of SEQ ID NO: 95 is Gly, Ala, Val, Leu, Ile, Pro, Phe, Met, or Trp: (cc) the amino acid sequence of SEQ ID NO: 96 (predicted sequence of β chain of 1913 TCR-41BB without N-terminal signal peptide), wherein X at position 169 of SEQ ID NO: 96 is Ser or Cys: (dd) the amino acid sequences of both of SEQ ID NOs: 95 and 96: (ee) the amino acid sequence of SEQ ID NO: 97 (a chain of cysteine-substituted, LVL-modified 1913 TCR-41BB with N-terminal signal peptide): (ff) the amino acid sequence of SEQ ID NO: 98 (β chain of cysteine-substituted. LVL-modified 1913 TCR-41BB with N-terminal signal peptide): (gg) the amino acid sequence of SEQ ID NO: 99 (predicted sequence of α chain of cysteine-substituted, LVL-modified 1913 TCR-41BB without N-terminal signal peptide): (hh) the amino acid sequence of SEQ ID NO: 100 (predicted sequence of β chain of cysteine-substituted, LVL-modified 1913 TCR-41BB without N-terminal signal peptide); (ii) the amino acid sequences of both of SEQ ID NOs: 97 and 98: (jj) the amino acid sequences of both of SEQ ID NOs: 99 and 100; (kk) the amino acid sequence of SEQ ID NO: 101 (a chain of 4286 TCR 10-1 with N-terminal signal peptide), wherein: (i) X at position 176 of SEQ ID NO: 101 is Thr or Cys: (ii) X at position 240 of SEQ ID NO: 101 is Ser, Ala, Val, Leu, Ile, Pro, Phe. Met, or Trp: (iii) X at position 242 of SEQ ID NO: 101 is Met. Ala. Val. Leu. Ile. Pro. Phe, or Trp; and (iv) X at position 243 of SEQ ID NO: 101 is Gly, Ala. Val, Leu, Ile, Pro, Phe, Met, or Trp: (11) the amino acid sequence of SEQ ID NO: 102 (β chain of 4286 TCR 10-1 with N-terminal signal peptide), wherein X at position 190 of SEQ ID NO: 102 is Ser or Cys: (mm) the amino acid sequences of both of SEQ ID NOs: 101 and 102: (nn) the amino acid sequence of SEQ ID NO: 103 (predicted sequence of α chain of 4286 TCR 10-1 without N-terminal signal peptide), wherein: (i) X at position 156 of SEQ ID NO: 103 is Thr or Cys: (ii) X at position 220 of SEQ ID NO: 103 is Ser, Ala, Val, Leu, Ile, Pro, Phe, Met, or Trp: (iii) X at position 222 of SEQ ID NO: 103 is Met, Ala. Val, Leu, Ile, Pro, Phe, or Trp; and (iv) X at position 223 of SEQ ID NO: 103 is Gly. Ala, Val. Leu. Ile. Pro, Phe. Met, or Trp: (00) the amino acid sequence of SEQ ID NO: 104 (predicted sequence of β chain of 4286 TCR 10-1 without N-terminal signal peptide), wherein X at position 169 of SEQ ID NO: 104 is Ser or Cys: (pp) the amino acid sequences of both of SEQ ID NOs: 103 and 104; (qq) the amino acid sequence of SEQ ID NO: 105 (a chain of cysteine-substituted. LVL-modified 4286 TCR 10-1 with N-terminal signal peptide); (rr) the amino acid sequence of SEQ ID NO: 106 (β chain of cysteine-substituted. LVL-modified 4286 TCR 10-1 with N-terminal signal peptide): (ss) the amino acid sequence of SEQ ID NO: 107 (predicted sequence of α chain of cysteine-substituted, LVL-modified 4286 TCR 10-1 without N-terminal signal peptide): (tt) the amino acid sequence of SEQ ID NO: 108 (predicted sequence of β chain of cysteine-substituted, LVL-modified 4286 TCR 10-1 without N-terminal signal peptide): (uu) the amino acid sequences of both of SEQ ID NOs: 105 and 106: (vv) the amino acid sequences of both of SEQ ID NOs: 107 and 108: (ww) the amino acid sequence of SEQ ID NO: 109 (a chain of 4286 TCR 10-2 with N-terminal signal peptide), wherein: (i) X at position 180 of SEQ ID NO: 109 is Thr or Cys: (ii) X at position 244 of SEQ ID NO: 109 is Ser, Ala. Val, Leu. Ile. Pro. Phe. Met, or Trp: (iii) X at position 246 of SEQ ID NO: 109 is Met. Ala. Val. Leu. Ile. Pro. Phe, or Trp; and (iv) X at position 247 of SEQ ID NO: 109 is Gly, Ala. Val, Leu, Ile, Pro, Phe, Met, or Trp: (xx) the amino acid sequence of SEQ ID NO: 110 (chain of 4286 TCR 10-2 with N-terminal signal peptide), wherein X at position 192 of SEQ ID NO: 110 is Ser or Cys: (vy) the amino acid sequences of both of SEQ ID NOs: 109 and 110: (zz) the amino acid sequence of SEQ ID NO: 111 (predicted sequence of α chain of 4286 TCR 10-2 without N-terminal signal peptide), wherein: (i) X at position 160 of SEQ ID NO: 111 is Thr or Cys: (ii) X at position 224 of SEQ ID NO: 111 is Ser, Ala, Val, Leu, Ile, Pro, Phe, Met, or Trp: (iii) X at position 226 of SEQ ID NO: 111 is Met, Ala. Val, Leu. Ile. Pro, Phe, or Trp; and (iv) X at position 227 of SEQ ID NO: 111 is Gly. Ala. Val. Leu. Ile. Pro. Phe. Met, or Trp: (aaa) the amino acid sequence of SEQ ID NO: 112 (predicted sequence of β chain of 4286 TCR 10-2 without N-terminal signal peptide), wherein X at position 171 of SEQ ID NO: 112 is Ser or Cys: (bbb) the amino acid sequences of both of SEQ ID NOs: 111 and 112: (ccc) the amino acid sequence of SEQ ID NO: 113 (a chain of cysteine-substituted, LVL-modified 4286 TCR 10-2 with N-terminal signal peptide): (ddd) the amino acid sequence of SEQ ID NO: 114 (β chain of cysteine-substituted, LVL-modified 4286 TCR 10-2 with N-terminal signal peptide): (eee) the amino acid sequence of SEQ ID NO: 115 (predicted sequence of α chain of cysteine-substituted, LVL-modified 4286 TCR 10-2 without N-terminal signal peptide): (fff) the amino acid sequence of SEQ ID NO: 116 (predicted sequence of β chain of cysteine-substituted, LVL-modified 4286 TCR 10-2 without N-terminal signal peptide): (ggg) the amino acid sequences of both of SEQ ID NOs: 113 and 114: (hhh) the amino acid sequences of both of SEQ ID NOs: 115 and 116; (iii) the amino acid sequence of SEQ ID NO: 117 (a chain of 4286 TCR 10-3 with N-terminal signal peptide), wherein: (i) X at position 180 of SEQ ID NO: 117 is Thr or Cys: (ii) X at position 244 of SEQ ID NO: 117 is Ser, Ala. Val, Leu. Ile. Pro, Phe. Met, or Trp: (iii) X at position 246 of SEQ ID NO: 117 is Met, Ala. Val, Leu. Ile. Pro, Phe, or Trp; and (iv) X at position 247 of SEQ ID NO: 117 is Gly, Ala, Val, Leu. Ile. Pro, Phe. Met, or Trp: (jjj) the amino acid sequence of SEQ ID NO: 118 (β chain of 4286 TCR 10-3 with N-terminal signal peptide), wherein X at position 198 of SEQ ID NO: 118 is Ser or Cys: (kkk) the amino acid sequences of both of SEQ ID NOs: 117 and 118: (III) the amino acid sequence of SEQ ID NO: 119 (predicted sequence of α chain of 4286 TCR 10-3 without N-terminal signal peptide), wherein: (i) X at position 160 of SEQ ID NO: 119 is Thr or Cys: (ii) X at position 224 of SEQ ID NO: 119 is Ser, Ala. Val, Leu, Ile, Pro, Phe, Met, or Trp: (iii) X at position 226 of SEQ ID NO: 119 is Met. Ala. Val. Leu. Ile. Pro. Phe, or Trp; and (iv) X at position 227 of SEQ ID NO: 119 is Gly, Ala. Val, Leu, Ile, Pro, Phe, Met, or Trp: (mmm) the amino acid sequence of SEQ ID NO: 120 (predicted sequence of β chain of 4286 TCR 10-3 without N-terminal signal peptide), wherein X at position 177 of SEQ ID NO: 120 is Ser or Cys: (nnn) the amino acid sequences of both of SEQ ID NOs: 119 and 120: (000) the amino acid sequence of SEQ ID NO: 121 (a chain of cysteine-substituted. LVL-modified 4286 TCR 10-3 with N-terminal signal peptide): (ppp) the amino acid sequence of SEQ ID NO: 122 (B chain of cysteine-substituted, LVL-modified 4286 TCR 10-3 with N-terminal signal peptide): (qqq) the amino acid sequence of SEQ ID NO: 123 (predicted sequence of α chain of cysteine-substituted. LVL-modified 4286 TCR 10-3 without N-terminal signal peptide); (rrr) the amino acid sequence of SEQ ID NO: 124 (predicted sequence of β chain of cysteine-substituted, LVL-modified 4286 TCR 10-3 without N-terminal signal peptide): (sss) the amino acid sequences of both of SEQ ID NOs: 121 and 122: (ttt) the amino acid sequences of both of SEQ ID NOs: 123 and 124: (uuu) the amino acid sequence of SEQ ID NO: 125 (a chain of 4286 TCR 10-4 with N-terminal signal peptide), wherein: (i) X at position 180 of SEQ ID NO: 125 is Thr or Cys: (ii) X at position 244 of SEQ ID NO: 125 is Ser, Ala, Val, Leu, Ile, Pro, Phe, Met, or Trp: (iii) X at position 246 of SEQ ID NO: 125 is Met, Ala, Val, Leu, Ile, Pro. Phe, or Trp; and (iv) X at position 247 of SEQ ID NO: 125 is Gly, Ala. Val, Leu. Ile. Pro, Phe. Met, or Trp: (vvv) the amino acid sequence of SEQ ID NO: 126 (β chain of 4286 TCR 10-4 with N-terminal signal peptide), wherein X at position 198 of SEQ ID NO: 126 is Ser or Cys: (www) the amino acid sequences of both of SEQ ID NOs: 125 and 126: (xxx) the amino acid sequence of SEQ ID NO: 127 (predicted sequence of α chain of 4286 TCR 10-4 without N-terminal signal peptide), wherein: (i) X at position 160 of SEQ ID NO: 127 is Thr or Cys: (ii) X at position 224 of SEQ ID NO: 127 is Ser, Ala, Val, Leu, Ile, Pro, Phe, Met, or Trp: (iii) X at position 226 of SEQ ID NO: 127 is Met. Ala, Val, Leu. Ile. Pro, Phe, or Trp; and (iv) X at position 227 of SEQ ID NO: 127 is Gly. Ala, Val, Leu, Ile, Pro, Phe, Met, or Trp: (yyy) the amino acid sequence of SEQ ID NO: 128 (predicted sequence of β chain of 4286 TCR 10-4 without N-terminal signal peptide), wherein X at position 177 of SEQ ID NO: 128 is Ser or Cys: (zzz) the amino acid sequences of both of SEQ ID NOs: 127 and 128: (aaaa) the amino acid sequence of SEQ ID NO: 129 (a chain of cysteine-substituted, LVL-modified 4286 TCR 10-4 with N-terminal signal peptide): (bbbb) the amino acid sequence of SEQ ID NO: 130 (β chain of cysteine-substituted, LVL-modified 4286 TCR 10-4 with N-terminal signal peptide): (cccc) the amino acid sequence of SEQ ID NO: 131 (predicted sequence of α chain of cysteine-substituted. LVL-modified 4286 TCR 10-4 without N-terminal signal peptide): (dddd) the amino acid sequence of SEQ ID NO: 132 (predicted sequence of β chain of cysteine-substituted, LVL-modified 4286 TCR 10-4 without N-terminal signal peptide): (eeee) the amino acid sequences of both of SEQ ID NOS: 129 and 130; or (ffff) the amino acid sequences of both of SEQ ID NOs: 131 and 132.

In an aspect of the invention, the substituted constant region includes cysteine substitutions in the constant region of one or both of the α and β chains to provide a cysteine-substituted TCR. Opposing cysteines in the a and the β chains provide a disulfide bond that links the constant regions of the a and the β chains of the substituted TCR to one another and which is not present in a TCR comprising the unsubstituted murine constant regions. In this regard, the TCR may be a cysteine-substituted TCR in which one or both of the native Thr at position 48 (Thr48) of SEQ ID NO: 75 and the native Ser at position 57 (Ser57) of SEQ ID NO: 76 may be substituted with Cys. Preferably, both of the native Thr48 of SEQ ID NO: 75 and the native Ser57 of SEQ ID NO: 76 are substituted with Cys. Examples of cysteine-substituted TCR constant regions sequences are set forth in Table 1. In an aspect of the invention, the cysteine-substituted TCR comprises (i) SEQ ID NO: 71, (ii) SEQ ID NO: 72, or (iii) both of SEQ ID NOs: 71 and 72, wherein both of SEQ ID NOs: 71 and 72 are as defined in Table 1. The cysteine-substituted TCRs of the invention may include the substituted constant region in addition to any of the CDRs or variable regions described herein.

In an aspect of the invention, the cysteine-substituted, chimeric TCR comprises a full length a chain and a full-length β chain. Examples of cysteine-substituted, chimeric TCR a chain and β chain sequences are set forth in Table 1. In an aspect of the invention, the TCR comprises: (1) SEQ ID NO: 77, (2) SEQ ID NO: 78, (3) SEQ ID NO: 79. (4) SEQ ID NO: 80, (5) SEQ ID NO: 85, (6) SEQ ID NO: 86, (7) SEQ ID NO: 87, (8) SEQ ID NO: 88, (9) SEQ ID NO: 93, (10) SEQ ID NO: 94, (11) SEQ ID NO: 95, (12) SEQ ID NO: 96, (13) SEQ ID NO: 101, (14) SEQ ID NO: 102, (15) SEQ ID NO: 103, (16) SEQ ID NO: 104, (17) SEQ ID NO: 109, (18) SEQ ID NO: 110, (19) SEQ ID NO: 111, (20) SEQ ID NO: 112, (21) SEQ ID NO: 117, (22) SEQ ID NO: 118, (23) SEQ ID NO: 119, (24) SEQ ID NO: 120, (25) SEQ ID NO: 125, (26) SEQ ID NO: 126, (27) SEQ ID NO: 127, (28) SEQ ID NO: 128, (29) both of SEQ ID NOs: 77 and 78, (30) both of SEQ ID NOs: 79 and 80, (31) both of SEQ ID NOs: 85 and 86, (32) both of SEQ ID NOs: 87 and 88, (33) both of SEQ ID NOs: 93 and 94, (34) both of SEQ ID NOs: 95 and 96, (35) both of SEQ ID NOs: 101 and 102, (36) both of SEQ ID NOs: 103 and 104, (37) both of SEQ ID NOs: 109 and 110, (38) both of SEQ ID NOS: 111 and 112, (39) both of SEQ ID NOs: 117 and 118, (40) both of SEQ ID NOs: 119 and 120, (41) both of SEQ ID NOs: 125 and 126, or (42) both of SEQ ID NOs: 127 and 128, wherein all of SEQ ID NOs: 77-80, 85-88, 93-96, 101-104, 109-112, 117-120, and 125-128 are as defined in Table 1.

TABLE 1

| SEQ ID NO: | Definitions of “X” |
|---|---|
| SEQ ID NO: 71 | X at position 48 is Cys, |
| (constant region α chain) | X at position 112 is Ser, |
| | X at position 114 is Met and |
| | X at position 115 is Gly. |
| SEQ ID NO: 72 | X at position 57 is Cys |
| (constant region β chain) | |
| SEQ ID NO: 77 | X at position 182 is Cys, |
| (3333 TCR-A α chain) (with N- | X at position 246 is Ser, |
| terminal signal peptide) | X at position 248 is Met and |
| | X at position 249 is Gly. |
| SEQ ID NO: 78 | X at position 188 is Cys |
| (3333 TCR-A β chain) (with N- | |
| terminal signal peptide) | |
| SEQ ID NO: 79 | X at position 161 is Cys, |
| (3333 TCR-A α chain) (predicted | X at position 225 is Ser, |
| sequence without N-terminal signal | X at position 227 is Met and |
| peptide) | X at position 228 is Gly. |
| SEQ ID NO: 80 | X at position 166 is Cys |
| (3333 TCR-A β chain) (predicted | |
| sequence without N-terminal signal | |
| peptide) | |
| SEQ ID NO: 85 | X at position 184 is Cys, |
| (3333 TCR-C α chain) (with N- | X at position 248 is Ser, |
| terminal signal peptide) | X at position 250 is Met and |
| | X at position 251 is Gly. |
| SEQ ID NO: 86 | X at position 188 is Cys |
| (3333 TCR-C β chain) (with N- | |
| terminal signal peptide) | |
| SEQ ID NO: 87 | X at position 163 is Cys, |
| (3333 TCR-C α chain) (predicted | X at position 227 is Ser, |
| sequence without N-terminal signal | X at position 229 is Met and |
| peptide) | X at position 230 is Gly. |
| SEQ ID NO: 88 | X at position 167 is Cys |
| (3333 TCR-C β chain) (predicted | |
| sequence without N-terminal signal | |
| peptide) | |
| SEQ ID NO: 93 | X at position 178 is Cys, |
| (1913 TCR-41BB α chain) (with N- | X at position 242 is Ser, |
| terminal signal peptide) | X at position 244 is Met and |
| | X at position 245 is Gly. |
| SEQ ID NO: 94 | X at position 190 is Cys |
| (1913 TCR-41BB β chain) (with N- | |
| terminal signal peptide) | |
| SEQ ID NO: 95 | X at position 160 is Cys, |
| (1913 TCR-41BB α chain) | X at position 224 is Ser, |
| (predicted sequence without N- | X at position 226 is Met and |
| terminal signal peptide) | X at position 227 is Gly. |
| SEQ ID NO: 96 | X at position 169 is Cys |
| (1913 TCR-41BB β chain) | |
| (predicted sequence without N- | |
| terminal signal peptide) | |
| SEQ ID NO: 101 | X at position 176 is Cys, |
| (4286 TCR 10-1 α chain) (with N- | X at position 240 is Ser, |
| terminal signal peptide) | X at position 242 is Met and |
| | X at position 243 is Gly. |
| SEQ ID NO: 102 | X at position 190 is Cys |
| (4286 TCR 10-1 β chain) (with N- | |
| terminal signal peptide) | |
| SEQ ID NO: 103 | X at position 156 is Cys, |
| (4286 TCR 10-1 α chain) (predicted | X at position 220 is Ser, |
| sequence without N-terminal signal | X at position 222 is Met and |
| peptide) | X at position 223 is Gly. |
| SEQ ID NO: 104 | X at position 169 is Cys |
| (4286 TCR 10-1 β chain) (predicted | |
| sequence without N-terminal signal | |
| peptide) | |
| SEQ ID NO: 109 | X at position 180 is Cys, |
| (4286 TCR 10-2 α chain) (with N- | X at position 244 is Ser, |
| terminal signal peptide) | X at position 246 is Met and |
| | X at position 247 is Gly. |
| SEQ ID NO: 110 | X at position 192 is Cys |
| (4286 TCR 10-2 β chain) (with N- | |
| terminal signal peptide) | |
| SEQ ID NO: 111 | X at position 160 is Cys, |
| (4286 TCR 10-2 α chain) (predicted | X at position 224 is Ser, |
| sequence without N-terminal signal | X at position 226 is Met and |
| peptide) | X at position 227 is Gly. |
| SEQ ID NO: 112 | X at position 171 is Cys |
| (4286 TCR 10-2 β chain) (predicted | |
| sequence without N-terminal signal | |
| peptide) | |
| SEQ ID NO: 117 | X at position 180 is Cys, |
| (4286 TCR 10-3 α chain) (with N- | X at position 244 is Ser, |
| terminal signal peptide) | X at position 246 is Met and |
| | X at position 247 is Gly. |
| SEQ ID NO: 118 | X at position 198 is Cys |
| (4286 TCR 10-3 β chain) (with N- | |
| terminal signal peptide) | |
| SEQ ID NO: 119 | X at position 160 is Cys, |
| (4286 TCR 10-3 α chain) (predicted | X at position 224 is Ser, |
| sequence without N-terminal signal | X at position 226 is Met and |
| peptide) | X at position 227 is Gly. |
| SEQ ID NO: 120 | X at position 177 is Cys |
| (4286 TCR 10-3 β chain) (predicted | |
| sequence without N-terminal signal | |
| peptide) | |
| SEQ ID NO: 125 | X at position 180 is Cys, |
| (4286 TCR 10-4 α chain) (with N- | X at position 244 is Ser, |
| terminal signal peptide) | X at position 246 is Met and |
| | X at position 247 is Gly. |
| SEQ ID NO: 126 | X at position 198 is Cys |
| (4286 TCR 10-4 β chain) (with N- | |
| terminal signal peptide) | |
| SEQ ID NO: 127 | X at position 160 is Cys, |
| (4286 TCR 10-4 α chain) (predicted | X at position 224 is Ser, |
| sequence without N-terminal signal | X at position 226 is Met and |
| peptide) | X at position 227 is Gly. |
| SEQ ID NO: 128 | X at position 177 is Cys |
| (4286 TCR 10-4 β chain) (predicted | |
| sequence without N-terminal signal | |
| peptide) | |

In an aspect of the invention, the substituted amino acid sequence includes substitutions of one, two, or three amino acids in the transmembrane (TM) domain of the constant region of the a chain with a hydrophobic amino acid to provide a hydrophobic amino acid-substituted TCR (also referred to herein as an “LVL-modified TCR”). The hydrophobic amino acid substitution(s) in the TM domain of the TCR may increase the hydrophobicity of the TM domain of the TCR as compared to a TCR that lacks the hydrophobic amino acid substitution(s) in the TM domain. In this regard, the TCR is an LVL-modified TCR in which one, two, or three of the native Ser112, Met114, and Gly 115 of SEQ ID NO: 75 may, independently, be substituted with Ala, Val, Leu, Ile, Pro, Phe. Met, or Trp; preferably with Leu, Ile, or Val. Preferably, all three of the native Serl 12, Met114, and Gly 115 of SEQ ID NO: 75 may, independently, be substituted with Ala, Val, Leu, Ile, Pro, Phe, Met, or Trp: preferably with Leu, Ile, or Val. In an aspect of the invention, the LVL-modified TCR comprises (i) SEQ ID NO: 71, (ii) SEQ ID NO: 72, or (iii) both of SEQ ID NOs: 71 and 72, wherein both of SEQ ID NOs: 71 and 72 are as defined in Table 2. The LVL-modified TCRs of the invention may include the substituted constant region in addition to any of the CDRs or variable regions described herein.

In an aspect of the invention, the LVL-modified TCR comprises a full length a chain and a full-length β chain. Examples of LVL-modified TCR a chain and βchain sequences are set forth in Table 2. In an aspect of the invention, the TCR comprises: (1) SEQ ID NO: 77, (2) SEQ ID NO: 78, (3) SEQ ID NO: 79, (4) SEQ ID NO: 80, (5) SEQ ID NO: 85, (6) SEQ ID NO: 86, (7) SEQ ID NO: 87, (8) SEQ ID NO: 88, (9) SEQ ID NO: 93, (10) SEQ ID NO: 94, (11) SEQ ID NO: 95, (12) SEQ ID NO: 96, (13) SEQ ID NO: 101, (14) SEQ ID NO: 102, (15) SEQ ID NO: 103, (16) SEQ ID NO: 104, (17) SEQ ID NO: 109, (18) SEQ ID NO: 110, (19) SEQ ID NO: 111, (20) SEQ ID NO: 112, (21) SEQ ID NO: 117, (22) SEQ ID NO: 118, (23) SEQ ID NO: 119, (24) SEQ ID NO: 120, (25) SEQ ID NO: 125, (26) SEQ ID NO: 126, (27) SEQ ID NO: 127, (28) SEQ ID NO: 128, (29) both of SEQ ID NOS: 77 and 78, (30) both of SEQ ID NOs: 79 and 80, (31) both of SEQ ID NOs: 85 and 86, (32) both of SEQ ID NOs: 87 and 88, (33) both of SEQ ID NOs: 93 and 94, (34) both of SEQ ID NOs: 95 and 96, (35) both of SEQ ID NOs: 101 and 102, (36) both of SEQ ID NOs: 103 and 104, (37) both of SEQ ID NOs: 109 and 110, (38) both of SEQ ID NOs: 111 and 112, (39) both of SEQ ID NOs: 117 and 118, (40) both of SEQ ID NOs: 119 and 120, (41) both of SEQ ID NOs: 125 and 126, or (42) both of SEQ ID NOs: 127 and 128, wherein all of SEQ ID NOs: 77-80, 85-88, 93-96, 101-104, 109-112, 117-120, and 125-128 are as defined in Table 2.

TABLE 2

| SEQ ID NO: | Definitions of “X” |
|---|---|
| SEQ ID NO: 71 | X at position 48 is Thr; |
| (constant region α chain) | X at position 112 is Ser, Ala, Val, Leu, Ile, Pro, Phe, Met, or Trp; |
| | preferably wherein X at position 112 is Leu, Ile, or Val; |
| | especially preferably wherein X at position 112 is Leu; |
| | X at position 114 is Met, Ala, Val, Leu, Ile, Pro, Phe, or Trp; |
| | preferably wherein X at position 114 is Leu, Ile, or Val; |
| | especially preferably wherein X at position 114 is Ile; and |
| | X at position 115 is Gly, Ala, Val, Leu, Ile, Pro, Phe, Met, or Trp; |
| | preferably wherein X at position 115 is Leu, Ile, or Val; |
| | especially preferably wherein X at position 115 is Val; |
| | Wherein SEQ ID NO: 71 does not comprise SEQ ID NO: 75 |
| | (unsubstituted constant region of α chain) |
| SEQ ID NO: 72 | X at position 57 is Ser |
| (constant region β chain) | |
| SEQ ID NO: 77 | X at position 182 is Thr; |
| (3333 TCR-A α chain) (with N- | X at position 246 is Ser, Ala, Val, Leu, Ile, Pro, Phe, Met, or Trp; |
| terminal signal peptide) | preferably wherein X at position 246 is Leu, Ile, or Val; |
| | especially preferably wherein X at position 246 is Leu; |
| | X at position 248 is Met, Ala, Val, Leu, Ile, Pro, Phe, or Trp; |
| | preferably wherein X at position 248 is Leu, Ile, or Val; |
| | especially preferably wherein X at position 248 is Ile; and |
| | X at position 249 is Gly, Ala, Val, Leu, Ile, Pro, Phe, Met, or Trp; |
| | preferably wherein X at position 249 is Leu, Ile, or Val; |
| | especially preferably wherein X at position 249 is Val, |
| | Wherein SEQ ID NO: 77 does not comprise SEQ ID NO: 75 |
| | (unsubstituted 3333 TCR-A α chain) |
| SEQ ID NO: 78 | X at position 188 is Ser |
| (3333 TCR-A β chain) (with N- | |
| terminal signal peptide) | |
| SEQ ID NO: 79 | X at position 161 is Thr; |
| (3333 TCR-A α chain) | X at position 225 is Ser, Ala, Val, Leu, Ile, Pro, Phe, Met, or Trp; |
| (predicted sequence without | preferably wherein X at position 225 is Leu, Ile, or Val; |
| N-terminal signal peptide) | especially preferably wherein X at position 225 is Leu; |
| | X at position 227 is Met, Ala, Val, Leu, Ile, Pro, Phe, or Trp; |
| | preferably wherein X at position 227 is Leu, Ile, or Val; |
| | especially preferably wherein X at position 227 is Ile; and |
| | X at position 228 is Gly, Ala, Val, Leu, Ile, Pro, Phe, Met, or Trp; |
| | preferably wherein X at position 228 is Leu, Ile, or Val; |
| | especially preferably wherein X at position 228 is Val, |
| | Wherein SEQ ID NO: 79 does not comprise SEQ ID NO: 75 |
| | (unsubstituted 3333 TCR-A α chain) |
| SEQ ID NO: 80 | X at position 166 is Ser |
| (3333 TCR-A β chain) | |
| (predicted sequence without | |
| N-terminal signal peptide) | |
| SEQ ID NO: 85 | X at position 184 is Thr; |
| (3333 TCR-C α chain) (with N- | X at position 248 is Ser, Ala, Val, Leu, Ile, Pro, Phe, Met, or Trp; |
| terminal signal peptide) | preferably wherein X at position 248 is Leu, Ile, or Val; |
| | especially preferably wherein X at position 248 is Leu; |
| | X at position 250 is Met, Ala, Val, Leu, Ile, Pro, Phe, or Trp; |
| | preferably wherein X at position 250 is Leu, Ile, or Val; |
| | especially preferably wherein X at position 250 is Ile; and |
| | X at position 251 is Gly, Ala, Val, Leu, Ile, Pro, Phe, Met, or Trp; |
| | preferably wherein X at position 251 is Leu, Ile, or Val; |
| | especially preferably wherein X at position 251 is Val, |
| | Wherein SEQ ID NO: 85 does not comprise SEQ ID NO: 75 |
| | (unsubstituted 3333 TCR-C α chain) |
| SEQ ID NO: 86 | X at position 188 is Ser |
| (3333 TCR-C β chain) (with N- | |
| terminal signal peptide) | |
| SEQ ID NO: 87 | X at position 163 is Thr; |
| (3333 TCR-C α chain) | X at position 227 is Ser, Ala, Val, Leu, Ile, Pro, Phe, Met, or Trp; |
| (predicted sequence without | preferably wherein X at position 227 is Leu, Ile, or Val; |
| N-terminal signal peptide) | especially preferably wherein X at position 227 is Leu; |
| | X at position 229 is Met, Ala, Val, Leu, Ile, Pro, Phe, or Trp; |
| | preferably wherein X at position 229 is Leu, Ile, or Val; |
| | especially preferably wherein X at position 229 is Ile; and |
| | X at position 230 is Gly, Ala, Val, Leu, Ile, Pro, Phe, Met, or Trp; |
| | preferably wherein X at position 230 is Leu, Ile, or Val; |
| | especially preferably wherein X at position 230 is Val, |
| | Wherein SEQ ID NO: 87 does not comprise SEQ ID NO: 75 |
| | (unsubstituted 3333 TCR-C α chain) |
| SEQ ID NO: 88 | X at position 167 is Ser |
| (3333 TCR-C β chain) | |
| (predicted sequence without | |
| N-terminal signal peptide) | |
| SEQ ID NO: 93 | X at position 178 is Thr; |
| (1913 TCR-41BB α chain) | X at position 242 is Ser, Ala, Val, Leu, Ile, Pro, Phe, Met, or Trp; |
| (with N-terminal signal | preferably wherein X at position 242 is Leu, Ile, or Val; |
| peptide) | especially preferably wherein X at position 242 is Leu; |
| | X at position 244 is Met, Ala, Val, Leu, Ile, Pro, Phe, or Trp; |
| | preferably wherein X at position 244 is Leu, Ile, or Val; |
| | especially preferably wherein X at position 244 is Ile; and |
| | X at position 245 is Gly, Ala, Val, Leu, Ile, Pro, Phe, Met, or Trp; |
| | preferably wherein X at position 245 is Leu, Ile, or Val; |
| | especially preferably wherein X at position 245 is Val, |
| | Wherein SEQ ID NO: 93 does not comprise SEQ ID NO: 75 |
| | (unsubstituted 1913 TCR-41BB α chain) |
| SEQ ID NO: 94 | X at position 190 is Ser |
| (1913 TCR-41BB β chain) | |
| (with N-terminal signal | |
| peptide) | |
| SEQ ID NO: 95 | X at position 160 is Thr; |
| (1913 TCR-41BB α chain) | X at position 224 is Ser, Ala, Val, Leu, Ile, Pro, Phe, Met, or Trp; |
| (predicted sequence without | preferably wherein X at position 224 is Leu, Ile, or Val; |
| N-terminal signal peptide) | especially preferably wherein X at position 224 is Leu; |
| | X at position 226 is Met, Ala, Val, Leu, Ile, Pro, Phe, or Trp; |
| | preferably wherein X at position 226 is Leu, Ile, or Val; |
| | especially preferably wherein X at position 226 is Ile; and |
| | X at position 227 is Gly, Ala, Val, Leu, Ile, Pro, Phe, Met, or Trp; |
| | preferably wherein X at position 227 is Leu, Ile, or Val; |
| | especially preferably wherein X at position 227 is Val, |
| | Wherein SEQ ID NO: 95 does not comprise SEQ ID NO: 75 |
| | (unsubstituted 1913 TCR-41BB α chain) |
| SEQ ID NO: 96 | X at position 169 is Ser |
| (1913 TCR-41BB β chain) | |
| (predicted sequence without | |
| N-terminal signal peptide) | |
| SEQ ID NO: 101 | X at position 176 is Thr; |
| (4286 TCR 10-1 α chain) (with | X at position 240 is Ser, Ala, Val, Leu, Ile, Pro, Phe, Met, or Trp; |
| N-terminal signal peptide) | preferably wherein X at position 240 is Leu, Ile, or Val; |
| | especially preferably wherein X at position 240 is Leu; |
| | X at position 242 is Met, Ala, Val, Leu, Ile, Pro, Phe, or Trp; |
| | preferably wherein X at position 242 is Leu, Ile, or Val; |
| | especially preferably wherein X at position 242 is Ile; and |
| | X at position 243 is Gly, Ala, Val, Leu, Ile, Pro, Phe, Met, or Trp; |
| | preferably wherein X at position 243 is Leu, Ile, or Val; |
| | especially preferably wherein X at position 243 is Val, |
| | Wherein SEQ ID NO: 101 does not comprise SEQ ID NO: 75 |
| | (unsubstituted 4286 TCR 10-1 α chain) |
| SEQ ID NO: 102 | X at position 190 is Ser |
| (4286 TCR 10-1 β chain) (with | |
| N-terminal signal peptide) | |
| SEQ ID NO: 103 | X at position 156 is Thr; |
| (4286 TCR 10-1 α chain) | X at position 220 is Ser, Ala, Val, Leu, Ile, Pro, Phe, Met, or Trp; |
| (predicted sequence without | preferably wherein X at position 220 is Leu, Ile, or Val; |
| N-terminal signal peptide) | especially preferably wherein X at position 220 is Leu; |
| | X at position 222 is Met, Ala, Val, Leu, Ile, Pro, Phe, or Trp; |
| | preferably wherein X at position 222 is Leu, Ile, or Val; |
| | especially preferably wherein X at position 222 is Ile; and |
| | X at position 223 is Gly, Ala, Val, Leu, Ile, Pro, Phe, Met, or Trp; |
| | preferably wherein X at position 223 is Leu, Ile, or Val; |
| | especially preferably wherein X at position 223 is Val, |
| | Wherein SEQ ID NO: 103 does not comprise SEQ ID NO: 75 |
| | (unsubstituted 4286 TCR 10-1 α chain) |
| SEQ ID NO: 104 | X at position 169 is Ser |
| (4286 TCR 10-1 β chain) | |
| (predicted sequence without | |
| N-terminal signal peptide) | |
| SEQ ID NO: 109 | X at position 180 is Thr; |
| (4286 TCR 10-2 α chain) (with | X at position 244 is Ser, Ala, Val, Leu, Ile, Pro, Phe, Met, or Trp; |
| N-terminal signal peptide) | preferably wherein X at position 244 is Leu, Ile, or Val; |
| | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; |
| | especially preferably wherein X at position 247 is Val, |
| | Wherein SEQ ID NO: 109 does not comprise SEQ ID NO: 75 |
| | (unsubstituted 4286 TCR 10-2 α chain) |
| SEQ ID NO: 110 | X at position 192 is Ser |
| (4286 TCR 10-2 β chain) (with | |
| N-terminal signal peptide) | |
| SEQ ID NO: 111 | X at position 160 is Thr; |
| (4286 TCR 10-2 α chain) | X at position 224 is Ser, Ala, Val, Leu, Ile, Pro, Phe, Met, or Trp; |
| (predicted sequence without | preferably wherein X at position 224 is Leu, Ile, or Val; |
| N-terminal signal peptide) | especially preferably wherein X at position 224 is Leu; |
| | X at position 226 is Met, Ala, Val, Leu, Ile, Pro, Phe, or Trp; |
| | preferably wherein X at position 226 is Leu, Ile, or Val; |
| | especially preferably wherein X at position 226 is Ile; and |
| | X at position 227 is Gly, Ala, Val, Leu, Ile, Pro, Phe, Met, or Trp; |
| | preferably wherein X at position 227 is Leu, Ile, or Val; |
| | especially preferably wherein X at position 227 is Val, |
| | Wherein SEQ ID NO: 111 does not comprise SEQ ID NO: 75 |
| | (unsubstituted 4286 TCR 10-2 α chain) |
| SEQ ID NO: 112 | X at position 171 is Ser |
| (4286 TCR 10-2 β chain) | |
| (predicted sequence without | |
| N-terminal signal peptide) | |
| SEQ ID NO: 117 | X at position 180 is Thr; |
| (4286 TCR 10-3 α chain) (with | X at position 244 is Ser, Ala, Val, Leu, Ile, Pro, Phe, Met, or Trp; |
| N-terminal signal peptide) | preferably wherein X at position 244 is Leu, Ile, or Val; |
| | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; |
| | especially preferably wherein X at position 247 is Val, |
| | Wherein SEQ ID NO: 117 does not comprise SEQ ID NO: 75 |
| | (unsubstituted 4286 TCR 10-3 α chain) |
| SEQ ID NO: 118 | X at position 198 is Ser |
| (4286 TCR 10-3 β chain) (with | |
| N-terminal signal peptide) | |
| SEQ ID NO: 119 | X at position 160 is Thr; |
| (4286 TCR 10-3 α chain) | X at position 224 is Ser, Ala, Val, Leu, Ile, Pro, Phe, Met, or Trp; |
| (predicted sequence without | preferably wherein X at position 224 is Leu, Ile, or Val; |
| N-terminal signal peptide) | especially preferably wherein X at position 224 is Leu; |
| | X at position 226 is Met, Ala, Val, Leu, Ile, Pro, Phe, or Trp; |
| | preferably wherein X at position 226 is Leu, Ile, or Val; |
| | especially preferably wherein X at position 226 is Ile; and |
| | X at position 227 is Gly, Ala, Val, Leu, Ile, Pro, Phe, Met, or Trp; |
| | preferably wherein X at position 227 is Leu, Ile, or Val; |
| | especially preferably wherein X at position 227 is Val, |
| | Wherein SEQ ID NO: 119 does not comprise SEQ ID NO: 75 |
| | (unsubstituted 4286 TCR 10-3 α chain) |
| SEQ ID NO: 120 | X at position 177 is Ser |
| (4286 TCR 10-3 β chain) | |
| (predicted sequence without | |
| N-terminal signal peptide) | |
| SEQ ID NO: 125 | X at position 180 is Thr; |
| (4286 TCR 10-4 α chain) (with | X at position 244 is Ser, Ala, Val, Leu, Ile, Pro, Phe, Met, or Trp; |
| N-terminal signal peptide) | preferably wherein X at position 244 is Leu, Ile, or Val; |
| | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; |
| | especially preferably wherein X at position 247 is Val, |
| | Wherein SEQ ID NO: 125 does not comprise SEQ ID NO: 75 |
| | (unsubstituted 4286 TCR 10-4 α chain) |
| SEQ ID NO: 126 | X at position 198 is Ser |
| (4286 TCR 10-4 β chain) (with | |
| N-terminal signal peptide) | |
| SEQ ID NO: 127 | X at position 160 is Thr; |
| (4286 TCR 10-4 α chain) | X at position 224 is Ser, Ala, Val, Leu, Ile, Pro, Phe, Met, or Trp; |
| (predicted sequence without | preferably wherein X at position 224 is Leu, Ile, or Val; |
| N-terminal signal peptide) | especially preferably wherein X at position 224 is Leu; |
| | X at position 226 is Met, Ala, Val, Leu, Ile, Pro, Phe, or Trp; |
| | preferably wherein X at position 226 is Leu, Ile, or Val; |
| | especially preferably wherein X at position 226 is Ile; and |
| | X at position 227 is Gly, Ala, Val, Leu, Ile, Pro, Phe, Met, or Trp; |
| | preferably wherein X at position 227 is Leu, Ile, or Val; |
| | especially preferably wherein X at position 227 is Val, |
| | Wherein SEQ ID NO: 127 does not comprise SEQ ID NO: 75 |
| | (unsubstituted 4286 TCR 10-4 α chain) |
| SEQ ID NO: 128 | X at position 177 is Ser |
| (4286 TCR 10-4 β chain) | |
| (predicted sequence without | |
| N-terminal signal peptide) | |

In an aspect of the invention, the substituted amino acid sequence includes the cysteine substitutions in the constant region of one or both of the α and β chains in combination with the substitution(s) of one, two, or three amino acids in the transmembrane (TM) domain of the constant region of the a chain with a hydrophobic amino acid (also referred to herein as “cysteine-substituted, LVL-modified TCR”). In this regard, the TCR is a cysteine-substituted, LVL-modified, chimeric TCR in which the native Thr48 of SEQ ID NO: 75 is substituted with Cys; one, two, or three of the native Ser112, Met114, and Glyl15 of SEQ ID NO: 75 are, independently, substituted with Ala, Val, Leu, Ile, Pro, Phe, Met, or Trp; preferably with Leu, Ile, or Val; and the native Ser57 of SEQ ID NO: 76 is substituted with Cys. Preferably, all three of the native Ser1 12, Met114, and Gly 115 of SEQ ID NO: 75 may, independently, be substituted with Ala, Val. Leu, Ile, Pro. Phe, Met, or Trp: preferably with Leu. Ile, or Val. In an aspect of the invention, the cysteine-substituted, I.VI-modified TCR comprises (i) SEQ ID NO: 71, (ii) SEQ ID NO: 72, or (iii) both of SEQ ID NOs: 71 and 72, wherein both of SEQ ID NOs: 71 and 72 are as defined in Table 3. The cysteine-substituted. LVL-modified TCRs of the invention may include the substituted constant region in addition to any of the CDRs or variable regions described herein.

In an aspect, the cysteine-substituted. LVL-modified TCR comprises a full-length α chain and a full-length β chain. Examples of cysteine-substituted, LVL-modified TCR αchain and β chain sequences are set forth in Tables 3 and 9. In an aspect of the invention, the TCR comprises: (1) SEQ ID NO: 77. (2) SEQ ID NO: 78. (3) SEQ ID NO: 79. (4) SEQ ID NO: 80. (5) SEQ ID NO: 81, (6) SEQ ID NO: 82, (7) SEQ ID NO: 83, (8) SEQ ID NO: 84, (9) SEQ ID NO: 85, (10) SEQ ID NO: 86, (11) SEQ ID NO: 87, (12) SEQ ID NO: 88, (13) SEQ ID NO: 89, (14) SEQ ID NO: 90. (15) SEQ ID NO: 91, (16) SEQ ID NO: 92, (17) SEQ ID NO: 93. (18) SEQ ID NO: 94, (19) SEQ ID NO: 95. (20) SEQ ID NO: 96, (21) SEQ ID NO: 97. (22) SEQ ID NO: 98, (23) SEQ ID NO: 99. (24) SEQ ID NO: 100. (25) SEQ ID NO: 101, (26) SEQ ID NO: 102, (27) SEQ ID NO: 103, (28) SEQ ID NO: 104, (29) SEQ ID NO: 105, (30) SEQ ID NO: 106, (31) SEQ ID NO: 107, (32) SEQ ID NO: 108, (33) SEQ ID NO: 109, (34) SEQ ID NO: 110, (35) SEQ ID NO: 111, (36) SEQ ID NO: 112, (37) SEQ ID NO: 113. (38) SEQ ID NO: 114. (39) SEQ ID NO: 115. (40) SEQ ID NO: 116. (41) SEQ ID NO: 117, (42) SEQ ID NO: 118, (43) SEQ ID NO: 119, (44) SEQ ID NO: 120, (45) SEQ ID NO: 121, (46) SEQ ID NO: 122, (47) SEQ ID NO: 123, (48) SEQ ID NO: 124, (49) SEQ ID NO: 125, (50) SEQ ID NO: 126, (51) SEQ ID NO: 127, (52) SEQ ID NO: 128, (53) SEQ ID NO: 129, (54) SEQ ID NO: 130, (55) SEQ ID NO: 131, (56) SEQ ID NO: 132, (57) both of SEQ ID NOs: 77 and 78. (58) both of SEQ ID NOs: 79 and 80, (59) both of SEQ ID NOs: 81 and 82, (60) both of SEQ ID NOs: 83 and 84, (61) both of SEQ ID NOs: 85 and 86. (62) both of SEQ ID NOs: 87 and 88. (63) both of SEQ ID NOs: 89 and 90, (64) both of SEQ ID NOs: 91 and 92. (65) both of SEQ ID NOs: 93 and 94, (66) both of SEQ ID NOs: 95 and 96, (67) both of SEQ ID NOs: 97 and 98. (68) both of SEQ ID NOs: 99 and 100, (69) both of SEQ ID NOs: 101 and 102, (70) both of SEQ ID NOs: 103 and 104, (71) both of SEQ ID NOs: 105 and 106, (72) both of SEQ ID NOs: 107 and 108, (73) both of SEQ ID NOs: 109 and 110, (74) both of SEQ ID NOs: 111 and 112, (75) both of SEQ ID NOs: 113 and 114, (76) both of SEQ ID NOs: 115 and 116. (77) both of SEQ ID NOs: 117 and 118. (78) both of SEQ ID NOs: 119 and 120, (79) both of SEQ ID NOs: 121 and 122, (80) both of SEQ ID NOs: 123 and 124. (81) both of SEQ ID NOs: 125 and 126. (82) both of SEQ ID NOs: 127 and 128, (83) both of SEQ ID NOs: 129 and 130, or (84) both of SEQ ID NOs: 131 and 132, wherein all of SEQ ID NOs: 77-80, 85-88, 93-96, 101-104, 109-112, 117-120, and 125-128 are as defined in Table 3.

TABLE 3

| SEQ ID NO: | Definitions of “X” |
|---|---|
| SEQ ID NO: 71 | X at position 48 is Cys; |
| (constant region α chain) | X at position 112 is Ser, Ala, Val, Leu, Ile, Pro, Phe, Met, or Trp; |
| | preferably wherein X at position 112 is Leu, Ile, or Val; |
| | especially preferably wherein X at position 112 is Leu; |
| | X at position 114 is Met, Ala, Val, Leu, Ile, Pro, Phe, or Trp; |
| | preferably wherein X at position 114 is Leu, Ile, or Val; |
| | especially preferably wherein X at position 114 is Ile; and |
| | X at position 115 is Gly, Ala, Val, Leu, Ile, Pro, Phe, Met, or Trp; |
| | preferably wherein X at position 115 is Leu, Ile, or Val; |
| | especially preferably wherein X at position 115 is Val |
| SEQ ID NO: 72 | X at position 57 is Cys |
| (constant region β chain) | |
| SEQ ID NO: 77 | X at position 182 is Cys; |
| (3333 TCR-A α chain) (with N- | X at position 246 is Ser, Ala, Val, Leu, Ile, Pro, Phe, Met, or Trp; |
| terminal signal peptide) | preferably wherein X at position 246 is Leu, Ile, or Val; |
| | especially preferably wherein X at position 246 is Leu; |
| | X at position 248 is Met, Ala, Val, Leu, Ile, Pro, Phe, or Trp; |
| | preferably wherein X at position 248 is Leu, Ile, or Val; |
| | especially preferably wherein X at position 248 is Ile; and |
| | X at position 249 is Gly, Ala, Val, Leu, Ile, Pro, Phe, Met, or Trp; |
| | preferably wherein X at position 249 is Leu, Ile, or Val; |
| | especially preferably wherein X at position 249 is Val |
| SEQ ID NO: 78 | X at position 188 is Cys |
| (3333 TCR-A β chain) (with N- | |
| terminal signal peptide) | |
| SEQ ID NO: 79 | X at position 161 is Cys; |
| (3333 TCR-A α chain) | X at position 225 is Ser, Ala, Val, Leu, Ile, Pro, Phe, Met, or Trp; |
| (predicted sequence without | preferably wherein X at position 225 is Leu, Ile, or Val; |
| N-terminal signal peptide) | especially preferably wherein X at position 225 is Leu; |
| | X at position 227 is Met, Ala, Val, Leu, Ile, Pro, Phe, or Trp; |
| | preferably wherein X at position 227 is Leu, Ile, or Val; |
| | especially preferably wherein X at position 227 is Ile; and |
| | X at position 228 is Gly, Ala, Val, Leu, Ile, Pro, Phe, Met, or Trp; |
| | preferably wherein X at position 228 is Leu, Ile, or Val; |
| | especially preferably wherein X at position 228 is Val |
| SEQ ID NO: 80 | X at position 166 is Cys |
| (3333 TCR-A β chain) | |
| (predicted sequence without | |
| N-terminal signal peptide) | |
| SEQ ID NO: 85 | X at position 184 is Cys; |
| (3333 TCR-C α chain) (with N- | X at position 248 is Ser, Ala, Val, Leu, Ile, Pro, Phe, Met, or Trp; |
| terminal signal peptide) | preferably wherein X at position 248 is Leu, Ile, or Val; |
| | especially preferably wherein X at position 248 is Leu; |
| | X at position 250 is Met, Ala, Val, Leu, Ile, Pro, Phe, or Trp; |
| | preferably wherein X at position 250 is Leu, Ile, or Val; |
| | especially preferably wherein X at position 250 is Ile; and |
| | X at position 251 is Gly, Ala, Val, Leu, Ile, Pro, Phe, Met, or Trp; |
| | preferably wherein X at position 251 is Leu, Ile, or Val; |
| | especially preferably wherein X at position 251 is Val |
| SEQ ID NO: 86 | X at position 188 is Cys |
| (3333 TCR-C β chain) (with N- | |
| terminal signal peptide) | |
| SEQ ID NO: 87 | X at position 163 is Cys; |
| (3333 TCR-C α chain) | X at position 227 is Ser, Ala, Val, Leu, Ile, Pro, Phe, Met, or Trp; |
| (predicted sequence without | preferably wherein X at position 227 is Leu, Ile, or Val; |
| N-terminal signal peptide) | especially preferably wherein X at position 227 is Leu; |
| | X at position 229 is Met, Ala, Val, Leu, Ile, Pro, Phe, or Trp; |
| | preferably wherein X at position 229 is Leu, Ile, or Val; |
| | especially preferably wherein X at position 229 is Ile; and |
| | X at position 230 is Gly, Ala, Val, Leu, Ile, Pro, Phe, Met, or Trp; |
| | preferably wherein X at position 230 is Leu, Ile, or Val; |
| | especially preferably wherein X at position 230 is Val |
| SEQ ID NO: 88 | X at position 167 is Cys |
| (3333 TCR-C β chain) | |
| (predicted sequence without | |
| N-terminal signal peptide) | |
| SEQ ID NO: 93 | X at position 178 is Cys; |
| (1913 TCR-41BB α chain) | X at position 242 is Ser, Ala, Val, Leu, Ile, Pro, Phe, Met, or Trp; |
| (with N-terminal signal | preferably wherein X at position 242 is Leu, Ile, or Val; |
| peptide) | especially preferably wherein X at position 242 is Leu; |
| | X at position 244 is Met, Ala, Val, Leu, Ile, Pro, Phe, or Trp; |
| | preferably wherein X at position 244 is Leu, Ile, or Val; |
| | especially preferably wherein X at position 244 is Ile; and |
| | X at position 245 is Gly, Ala, Val, Leu, Ile, Pro, Phe, Met, or Trp; |
| | preferably wherein X at position 245 is Leu, Ile, or Val; |
| | especially preferably wherein X at position 245 is Val |
| SEQ ID NO: 94 | X at position 190 is Cys |
| (1913 TCR-41BB β chain) | |
| (with N-terminal signal | |
| peptide) | |
| SEQ ID NO: 95 | X at position 160 is Cys; |
| (1913 TCR-41BB α chain) | X at position 224 is Ser, Ala, Val, Leu, Ile, Pro, Phe, Met, or Trp; |
| (predicted sequence without | preferably wherein X at position 224 is Leu, Ile, or Val; |
| N-terminal signal peptide) | especially preferably wherein X at position 224 is Leu; |
| | X at position 226 is Met, Ala, Val, Leu, Ile, Pro, Phe, or Trp; |
| | preferably wherein X at position 226 is Leu, Ile, or Val; |
| | especially preferably wherein X at position 226 is Ile; and |
| | X at position 227 is Gly, Ala, Val, Leu, Ile, Pro, Phe, Met, or Trp; |
| | preferably wherein X at position 227 is Leu, Ile, or Val; |
| | especially preferably wherein X at position 227 is Val |
| SEQ ID NO: 96 | X at position 169 is Cys |
| (1913 TCR-41BB β chain) | |
| (predicted sequence without | |
| N-terminal signal peptide) | |
| SEQ ID NO: 101 | X at position 176 is Cys; |
| (4286 TCR 10-1 α chain) (with | X at position 240 is Ser, Ala, Val, Leu, Ile, Pro, Phe, Met, or Trp; |
| N-terminal signal peptide) | preferably wherein X at position 240 is Leu, Ile, or Val; |
| | especially preferably wherein X at position 240 is Leu; |
| | X at position 242 is Met, Ala, Val, Leu, Ile, Pro, Phe, or Trp; |
| | preferably wherein X at position 242 is Leu, Ile, or Val; |
| | especially preferably wherein X at position 242 is Ile; and |
| | X at position 243 is Gly, Ala, Val, Leu, Ile, Pro, Phe, Met, or Trp; |
| | preferably wherein X at position 243 is Leu, Ile, or Val; |
| | especially preferably wherein X at position 243 is Val |
| SEQ ID NO: 102 | X at position 190 is Cys |
| (4286 TCR 10-1 β chain) (with | |
| N-terminal signal peptide) | |
| SEQ ID NO: 103 | X at position 156 is Cys; |
| (4286 TCR 10-1 α chain) | X at position 220 is Ser, Ala, Val, Leu, Ile, Pro, Phe, Met, or Trp; |
| (predicted sequence without | preferably wherein X at position 220 is Leu, Ile, or Val; |
| N-terminal signal peptide) | especially preferably wherein X at position 220 is Leu; |
| | X at position 222 is Met, Ala, Val, Leu, Ile, Pro, Phe, or Trp; |
| | preferably wherein X at position 222 is Leu, Ile, or Val; |
| | especially preferably wherein X at position 222 is Ile; and |
| | X at position 223 is Gly, Ala, Val, Leu, Ile, Pro, Phe, Met, or Trp; |
| | preferably wherein X at position 223 is Leu, Ile, or Val; |
| | especially preferably wherein X at position 223 is Val |
| SEQ ID NO: 104 | X at position 169 is Cys |
| (4286 TCR 10-1 β chain) | |
| (predicted sequence without | |
| N-terminal signal peptide) | |
| SEQ ID NO: 109 | X at position 180 is Cys; |
| (4286 TCR 10-2 α chain) (with | X at position 244 is Ser, Ala, Val, Leu, Ile, Pro, Phe, Met, or Trp; |
| N-terminal signal peptide) | preferably wherein X at position 244 is Leu, Ile, or Val; |
| | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; |
| | especially preferably wherein X at position 247 is Val |
| SEQ ID NO: 110 | X at position 192 is Cys |
| (4286 TCR 10-2 β chain) (with | |
| N-terminal signal peptide) | |
| SEQ ID NO: 111 | X at position 160 is Cys; |
| (4286 TCR 10-2 α chain) | X at position 224 is Ser, Ala, Val, Leu, Ile, Pro, Phe, Met, or Trp; |
| (predicted sequence without | preferably wherein X at position 224 is Leu, Ile, or Val; |
| N-terminal signal peptide) | especially preferably wherein X at position 224 is Leu; |
| | X at position 226 is Met, Ala, Val, Leu, Ile, Pro, Phe, or Trp; |
| | preferably wherein X at position 226 is Leu, Ile, or Val; |
| | especially preferably wherein X at position 226 is Ile; and |
| | X at position 227 is Gly, Ala, Val, Leu, Ile, Pro, Phe, Met, or Trp; |
| | preferably wherein X at position 227 is Leu, Ile, or Val; |
| | especially preferably wherein X at position 227 is Val |
| SEQ ID NO: 112 | X at position 171 is Cys |
| (4286 TCR 10-2 β chain) | |
| (predicted sequence without | |
| N-terminal signal peptide) | |
| SEQ ID NO: 117 | X at position 180 is Cys; |
| (4286 TCR 10-3 α chain) (with | X at position 244 is Ser, Ala, Val, Leu, Ile, Pro, Phe, Met, or Trp; |
| N-terminal signal peptide) | preferably wherein X at position 244 is Leu, Ile, or Val; |
| | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; |
| | especially preferably wherein X at position 247 is Val |
| SEQ ID NO: 118 | X at position 198 is Cys |
| (4286 TCR 10-3 β chain) (with | |
| N-terminal signal peptide) | |
| SEQ ID NO: 119 | X at position 160 is Cys; |
| (4286 TCR 10-3 α chain) | X at position 224 is Ser, Ala, Val, Leu, Ile, Pro, Phe, Met, or Trp; |
| (predicted sequence without | preferably wherein X at position 224 is Leu, Ile, or Val; |
| N-terminal signal peptide) | especially preferably wherein X at position 224 is Leu; |
| | X at position 226 is Met, Ala, Val, Leu, Ile, Pro, Phe, or Trp; |
| | preferably wherein X at position 226 is Leu, Ile, or Val; |
| | especially preferably wherein X at position 226 is Ile; and |
| | X at position 227 is Gly, Ala, Val, Leu, Ile, Pro, Phe, Met, or Trp; |
| | preferably wherein X at position 227 is Leu, Ile, or Val; |
| | especially preferably wherein X at position 227 is Val |
| SEQ ID NO: 120 | X at position 177 is Cys |
| (4286 TCR 10-3 β chain) | |
| (predicted sequence without | |
| N-terminal signal peptide) | |
| SEQ ID NO: 125 | X at position 180 is Cys; |
| (4286 TCR 10-4 α chain) (with | X at position 244 is Ser, Ala, Val, Leu, Ile, Pro, Phe, Met, or Trp; |
| N-terminal signal peptide) | preferably wherein X at position 244 is Leu, Ile, or Val; |
| | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; |
| | especially preferably wherein X at position 247 is Val |
| SEQ ID NO: 126 | X at position 198 is Cys |
| (4286 TCR 10-4 β chain) (with | |
| N-terminal signal peptide) | |
| SEQ ID NO: 127 | X at position 160 is Cys; |
| (4286 TCR 10-4 α chain) | X at position 224 is Ser, Ala, Val, Leu, Ile, Pro, Phe, Met, or Trp; |
| (predicted sequence without | preferably wherein X at position 224 is Leu, Ile, or Val; |
| N-terminal signal peptide) | especially preferably wherein X at position 224 is Leu; |
| | X at position 226 is Met, Ala, Val, Leu, Ile, Pro, Phe, or Trp; |
| | preferably wherein X at position 226 is Leu, Ile, or Val; |
| | especially preferably wherein X at position 226 is Ile; and |
| | X at position 227 is Gly, Ala, Val, Leu, Ile, Pro, Phe, Met, or Trp; |
| | preferably wherein X at position 227 is Leu, Ile, or Val; |
| | especially preferably wherein X at position 227 is Val |
| SEQ ID NO: 128 | X at position 177 is Cys |
| (4286 TCR 10-4 β chain) | |
| (predicted sequence without | |
| N-terminal signal peptide) | |

In an aspect of the invention, the cysteine-substituted, LVL-modified TCR comprises (a) SEQ ID NO: 73 (α chain constant region of cysteine-substituted, LVL-modified TCR); (b) SEQ ID NO: 74 (β chain constant region of cysteine-substituted. LVL-modified TCR); or (c) both (a) and (b).

Also provided by the invention is a polypeptide comprising a functional portion of any of the TCRs described herein. The term “polypeptide,” as used herein, includes oligopeptides and refers to a single chain of amino acids connected by one or more peptide bonds.

With respect to the inventive polypeptides, the functional portion can be any portion comprising contiguous amino acids of the TCR of which it is a part, provided that the functional portion specifically binds to mutated CDKN2A peptide. The term “functional portion,” when used in reference to a TCR, refers to any part or fragment of the TCR of the invention, which part or fragment retains the biological activity of the TCR of which it is a part (the parent TCR). Functional portions encompass, for example, those parts of a TCR that retain the ability to specifically bind to mutated CDKN2A peptide (e.g., within the context of any of the HLA Class I molecules described herein), or detect, treat, or prevent cancer, to a similar extent, the same extent, or to a higher extent, as the parent TCR. In reference to the parent TCR, the functional portion can comprise, for instance, about 10%, about 25%, about 30%, about 50%, about 70%, about 80%, about 90%, about 95%, or more, of the parent TCR.

The functional portion can comprise additional amino acids at the amino or carboxy terminus of the portion, or at both termini, which additional amino acids are not found in the amino acid sequence of the parent TCR. Desirably, the additional amino acids do not interfere with the biological function of the functional portion, e.g., specifically binding to mutated CDKN2A peptide; and/or having the ability to detect cancer, treat or prevent cancer, etc. More desirably, the additional amino acids enhance the biological activity, as compared to the biological activity of the parent TCR.

The polypeptide can comprise a functional portion of either or both of the α and βchains of the TCRs of the invention, such as a functional portion comprising one or more of the CDR1, CDR2, and CDR3 of the variable region(s) of the α chain and/or β chain of a TCR of the invention. In an aspect of the invention, the polypeptide can comprise the amino acid sequence of SEQ ID NO: 1 (CDR1 of α chain of 3333 TCR-A), SEQ ID NO: 2 (CDR2 of αchain of 3333 TCR-A), SEQ ID NO: 3 (CDR3 of α chain of 3333 TCR-A). SEQ ID NO: 4 (CDR1 of β chain of 3333 TCR-A). SEQ ID NO: 5 (CDR2 of β chain of 3333 TCR-A). SEQ ID NO: 6 (CDR3 of β chain of 3333 TCR-A), SEQ ID NO: 11 (CDR1 of α chain of 3333 TCR-C), SEQ ID NO: 12 (CDR2 of α chain of 3333 TCR-C), SEQ ID NO: 13 (CDR3 of αchain of 3333 TCR-C). SEQ ID NO: 14 (CDR1 of β chain of 3333 TCR-C), SEQ ID NO: 15 (CDR2 of β chain of 3333 TCR-C), SEQ ID NO: 16 (CDR3 of β chain of 3333 TCR-C). SEQ ID NO: 21 (CDR1 of α chain of 1913 TCR-41BB), SEQ ID NO: 22 (CDR2 of α chain of 1913 TCR-41BB), SEQ ID NO: 23 (CDR3 of α chain of 1913 TCR-41BB), SEQ ID NO: 24 (CDR1 of β chain of 1913 TCR-41BB), SEQ ID NO: 25 (CDR2 of β chain of 1913 TCR-41BB). SEQ ID NO: 26 (CDR3 of β chain of 1913 TCR-41BB), SEQ ID NO: 31 (CDR1 of α chain of 4286 TCR 10-1). SEQ ID NO: 32 (CDR2 of α chain of 4286 TCR 10-1). SEQ ID NO: 33 (CDR3 of α chain of 4286 TCR 10-1). SEQ ID NO: 34 (CDR1 of β chain of 4286 TCR 10-1). SEQ ID NO: 35 (CDR2 of β chain of 4286 TCR 10-1), SEQ ID NO: 36 (CDR3 of β chain of 4286 TCR 10-1). SEQ ID NO: 41 (CDR1 of α chain of 4286 TCR 10-2), SEQ ID NO: 42 (CDR2 of α chain of 4286 TCR 10-2). SEQ ID NO: 43 (CDR3 of α chain of 4286 TCR 10-2). SEQ ID NO: 44 (CDR1 of β chain of 4286 TCR 10-2), SEQ ID NO: 45 (CDR2 of β chain of 4286 TCR 10-2). SEQ ID NO: 46 (CDR3 of β chain of 4286 TCR 10-2), SEQ ID NO: 51 (CDR1 of α chain of 4286 TCR 10-3), SEQ ID NO: 52 (CDR2 of α chain of 4286 TCR 10-3). SEQ ID NO: 53 (CDR3 of α chain of 4286 TCR 10-3), SEQ ID NO: 54 (CDR1 of β chain of 4286 TCR 10-3), SEQ ID NO: 55 (CDR2 of β chain of 4286 TCR 10-3), SEQ ID NO: 56 (CDR3 of β chain of 4286 TCR 10-3), SEQ ID NO: 61 (CDR1 of α chain of 4286 TCR 10-4). SEQ ID NO: 62 (CDR2 of α chain of 4286 TCR 10-4), SEQ ID NO: 63 (CDR3 of α chain of 4286 TCR 10-4), SEQ ID NO: 64 (CDR1 of β chain of 4286 TCR 10-4), SEQ ID NO: 65 (CDR2 of β chain of 4286 TCR 10-4), SEQ ID NO: 66 (CDR3 of β chain of 4286 TCR 10-4), or a combination thereof. In this regard, the inventive polypeptide can comprise any one or more of the amino acid sequences selected from the group consisting of SEQ ID NOs: 1-6, 11-16, 21-26, 31-36, 41-46, 51-56, and 61-66. In an aspect of the invention, the polypeptide comprises the amino acid sequences of: (a) all of SEQ ID NOs: 1-3, (b) all of SEQ ID NOs: 4-6, (c) all of SEQ ID NOs: 1-6, (d) all of SEQ ID NOs: 11-13, (e) all of SEQ ID NOs: 14-16, (f) all of SEQ ID NOs: 11-16. (g) all of SEQ ID NOs: 21-23. (h) all of SEQ ID NOs: 24-26. (i) all of SEQ ID NOs: 21-26, (j) all of SEQ ID NOs: 31-33, (k) all of SEQ ID NOs: 34-36. (1) all of SEQ ID NOs: 31-36, (m) all of SEQ ID NOs: 41-43, (n) all of SEQ ID NOs: 44-46, (o) all of SEQ ID NOs: 41-46, (p) all of SEQ ID NOs: 51-53, (q) all of SEQ ID NOs: 54-56, (r) all of SEQ ID NOs: 51-56, (s) all of SEQ ID NOs: 61-63. (t) all of SEQ ID NOs: 64-66, or (u) all of SEQ ID NOs: 61-66. In a preferred aspect, the polypeptide comprises the amino acid sequences of all of (i) SEQ ID NOs: 1-6, (ii) SEQ ID NOs: 11-16, (iii) SEQ ID NOs: 21-26, (iv) SEQ ID NOs: 31-36, (v) SEQ ID NOs: 41-46, (vi) SEQ ID NOs: 51-56, or (vii) SEQ ID NOs: 61-66.

In an aspect of the invention, the inventive polypeptide can comprise. for instance, the variable region of the inventive TCR comprising a combination of the CDR regions set forth above. In this regard, the polypeptide can comprise the amino acid sequence(s) of (1) SEQ ID NO: 7. (2) SEQ ID NO: 8, (3) SEQ ID NO: 9, (4) SEQ ID NO: 10. (5) SEQ ID NO: 17, (6) SEQ ID NO: 18, (7) SEQ ID NO: 19, (8) SEQ ID NO: 20. (9) SEQ ID NO: 27, (10) SEQ ID NO: 28, (11) SEQ ID NO: 29. (12) SEQ ID NO: 30. (13) SEQ ID NO: 37, (14) SEQ ID NO: 38, (15) SEQ ID NO: 39, (16) SEQ ID NO: 40, (17) SEQ ID NO: 47. (18) SEQ ID NO: 48, (19) SEQ ID NO: 49, (20) SEQ ID NO: 50. (21) SEQ ID NO: 57, (22) SEQ ID NO: 58, (23) SEQ ID NO: 59, (24) SEQ ID NO: 60, (25) SEQ ID NO: 67. (26) SEQ ID NO: 68, (27) SEQ ID NO: 69. (28) SEQ ID NO: 70. (29) both of SEQ ID NOs: 7 and 8. (30) both of SEQ ID NOs: 9 and 10, (31) both of SEQ ID NOs: 17 and 18. (32) both of SEQ ID NOs: 19 and 20. (33) both of SEQ ID NOs: 27 and 28, (34) both of SEQ ID NOs: 29 and 30, (35) both of SEQ ID NOs: 37 and 38, (36) both of SEQ ID NOs: 39 and 40. (37) both of SEQ ID NOS: 47 and 48, (38) both of SEQ ID NOs: 49 and 50, (39) both of SEQ ID NOs: 57 and 58, (40) both of SEQ ID NOs: 59 and 60. (41) both of SEQ ID NOs: 67 and 68, or (42) both of SEQ ID NOs: 69 and 70. In a preferred aspect, the polypeptide comprises the amino acid sequences of (a) both of SEQ ID NOs: 7 and 8. (b) both of SEQ ID NOs: 9 and 10, (c) both of SEQ ID NOs: 17 and 18, (d) both of SEQ ID NOs: 19 and 20. (e) both of SEQ ID NOs: 27 and 28, (f) both of SEQ ID NOs: 29 and 30, (g) both of SEQ ID NOs: 37 and 38, (h) both of SEQ ID NOs: 39 and 40. (i) both of SEQ ID NOs: 47 and 48, (i) both of SEQ ID NOs: 49 and 50. (k) both of SEQ ID NOs: 57 and 58. (1) both of SEQ ID NOs: 59 and 60. (m) both of SEQ ID NOs: 67 and 68, or (n) both of SEQ ID NOs: 69 and 70.

In an aspect of the invention, the inventive polypeptide can further comprise the constant region of the inventive TCR set forth above. In this regard, the polypeptide can further comprise the amino acid sequence of SEQ ID NO: 75 (WT murine constant region of a chain), SEQ ID NO: 76 (WT murine constant region of β chain), SEQ ID NO: 71 (substituted murine constant region of α chain), SEQ ID NO: 72 (substituted murine constant region of β chain), SEQ ID NO: 73 (α chain constant region of cysteine-substituted, LVL-modified TCR): SEQ ID NO: 74 (β chain constant region of cysteine-substituted, LVL-modified TCR): both SEQ ID NOs: 71 and 72, both SEQ ID NOs: 73 and 74, or both SEQ ID NOs: 75 and 76. Preferably, the polypeptide further comprises the amino acid sequences of both of SEQ ID NOs: 71 and 72, both of SEQ ID NO: 73 and 74, or both of SEQ ID NOs: 75 and 76 in combination with any of the CDR regions or variable regions described herein with respect to other aspects of the invention. In an aspect of the invention, one or both of SEQ ID NOs: 71 and 72 of the polypeptide are as defined in any one of Tables 1-3.

In an aspect of the invention, the inventive polypeptide can comprise the entire length of an a or β chain of the TCR described herein. In this regard, the inventive polypeptide can comprise the amino acid sequence of SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95. SEQ ID NO: 96, SEQ ID NO: 97, SEQ ID NO: 98, SEQ ID NO: 99, SEQ ID NO: 100. SEQ ID NO: 101. SEQ ID NO: 102. SEQ ID NO: 103. SEQ ID NO: 104. SEQ ID NO: 105, SEQ ID NO: 106, SEQ ID NO: 107, SEQ ID NO: 108. SEQ ID NO: 109, SEQ ID NO: 110, SEQ ID NO: 111. SEQ ID NO: 112, SEQ ID NO: 113. SEQ ID NO: 114. SEQ ID NO: 115. SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123. SEQ ID NO: 124, SEQ ID NO: 125, SEQ ID NO: 126, SEQ ID NO: 127, SEQ ID NO: 128. SEQ ID NO: 129, SEQ ID NO: 130, SEQ ID NO: 131, or SEQ ID NO: 132. Alternatively, the polypeptide of the invention can comprise both chains of the TCRs described herein. For example, the polypeptide may comprise the amino acid sequences of: both of SEQ ID NOs: 77-78, both of SEQ ID NOs: 79-80, both of SEQ ID NOs: 81-82, both of SEQ ID NOs: 83-84, both of SEQ ID NOs: 85-86, both of SEQ ID NOs: 87-88, both of SEQ ID NOs: 89-90, both of SEQ ID NOs: 91-92, both of SEQ ID NOs: 93-94, both of SEQ ID NOs: 95-96, both of SEQ ID NOs: 97-98, both of SEQ ID NOs: 99-100, both of SEQ ID NOs: 101-102, both of SEQ ID NOs: 103-104, both of SEQ ID NOs: 105-106, both of SEQ ID NOs: 107-108, both of SEQ ID NOs: 109-110, both of SEQ ID NOs: 111-112, both of SEQ ID NOs: 113-114, both of SEQ ID NOs: 115-116, both of SEQ ID NOs: 117-118, both of SEQ ID NOs: 119-120, both of SEQ ID NOS: 121-122, both of SEQ ID NOs: 123-124, both of SEQ ID NOs: 125-126, both of SEQ ID NOs: 127-128, both of SEQ ID NOs: 129-130, or both of SEQ ID NOs: 131-132.

For example, the polypeptide of the invention can comprise (a) the amino acid sequence of SEQ ID NO: 77 (α chain of 3333 TCR-A with N-terminal signal peptide), wherein: (i) X at position 182 of SEQ ID NO: 77 is Thr or Cys: (ii) X at position 246 of SEQ ID NO: 77 is Ser. Ala, Val. Leu, Ile, Pro, Phe, Met, or Trp: (iii) X at position 248 of SEQ ID NO: 77 is Met, Ala, Val, Leu, Ile, Pro, Phe, or Trp; and (iv) X at position 249 of SEQ ID NO: 77 is Gly, Ala, Val, Leu, Ile, Pro, Phe, Met, or Trp: (b) the amino acid sequence of SEQ ID NO: 78 (β chain of 3333 TCR-A with N-terminal signal peptide), wherein X at position 188 of SEQ ID NO: 78 is Ser or Cys: (c) the amino acid sequences of both of SEQ ID NOs: 77 and 78: (d) the amino acid sequence of SEQ ID NO: 79 (predicted sequence of α chain of 3333 TCR-A without N-terminal signal peptide), wherein: (i) X at position 161 of SEQ ID NO: 79 is Thr or Cys: (ii) X at position 225 of SEQ ID NO: 79 is Ser. Ala, Val, Leu, Ile, Pro, Phe. Met, or Trp: (iii) X at position 227 of SEQ ID NO: 79 is Met. Ala, Val. Leu. Ile. Pro, Phe, or Trp; and (iv) X at position 228 of SEQ ID NO: 79 is Gly, Ala. Val, Leu, Ile, Pro, Phe, Met, or Trp: (e) the amino acid sequence of SEQ ID NO: 80) (predicted sequence of β chain of 3333 TCR-A without N-terminal signal peptide), wherein X at position 166 of SEQ ID NO: 80 is Ser or Cys: (f) the amino acid sequences of both of SEQ ID NOs: 79 and 80; (g) the amino acid sequence of SEQ ID NO: 81 (α chain of cysteine-substituted, LVL-modified 3333 TCR-A with N-terminal signal peptide): (h) the amino acid sequence of SEQ ID NO: 82 (β chain of cysteine-substituted, LVL-modified 3333 TCR-A with N-terminal signal peptide): (i) the amino acid sequence of SEQ ID NO: 83 (predicted sequence of α chain of cysteine-substituted. LVL-modified 3333 TCR-A without N-terminal signal peptide): (j) the amino acid sequence of SEQ ID NO: 84 (predicted sequence of β chain of cysteine-substituted, LVL-modified 3333 TCR-A without N-terminal signal peptide): (k) the amino acid sequences of both of SEQ ID NOs: 81 and 82: (1) the amino acid sequences of both of both of SEQ ID NOs: 83 and 84: (m) the amino acid sequence of SEQ ID NO: 85 (α chain of 3333 TCR-C with N-terminal signal peptide), wherein: (i) X at position 184 of SEQ ID NO: 85 is Thr or Cys: (ii) X at position 248 of SEQ ID NO: 85 is Ser. Ala. Val. Leu. Ile. Pro. Phe. Met. or Trp: (iii) X at position 250 of SEQ ID NO: 85 is Met. Ala, Val. Leu, Ile, Pro. Phe, or Trp; and (iv) X at position 251 of SEQ ID NO: 85 is Gly, Ala, Val, Leu, Ile, Pro, Phe, Met, or Trp: (n) the amino acid sequence of SEQ ID NO: 86 (β chain of 3333 TCR-C with N-terminal signal peptide), wherein X at position 188 of SEQ ID NO: 86 is Ser or Cys: (o) the amino acid sequences of both of SEQ ID NOs: 85 and 86: (p) the amino acid sequence of SEQ ID NO: 87 (predicted sequence of α chain of 3333 TCR-C without N-terminal signal peptide), wherein: (i) X at position 163 of SEQ ID NO: 87 is Thr or Cys: (ii) X at position 227 of SEQ ID NO: 87 is Ser, Ala, Val, Leu. Ile. Pro, Phe. Met, or Trp: (iii) X at position 229 of SEQ ID NO: 87 is Met. Ala. Val. Leu. Ile. Pro. Phe, or Trp; and (iv) X at position 230 of SEQ ID NO: 87 is Gly. Ala, Val. Leu, Ile, Pro, Phe, Met, or Trp; (q) the amino acid sequence of SEQ ID NO: 88 (predicted sequence of β chain of 3333 TCR-C without N-terminal signal peptide), wherein X at position 167 of SEQ ID NO: 88 is Ser or Cys; (r) the amino acid sequences of both of SEQ ID NOs: 87 and 88: (s) the amino acid sequence of SEQ ID NO: 89 (α chain of cysteine-substituted, LVL-modified 3333 TCR-C with N-terminal signal peptide): (t) the amino acid sequence of SEQ ID NO: 90 (β chain of cysteine-substituted, LVL-modified 3333 TCR-C with N-terminal signal peptide): (u) the amino acid sequence of SEQ ID NO: 91 (predicted sequence of α chain of cysteine-substituted, LVL-modified 3333 TCR-C without N-terminal signal peptide): (v) the amino acid sequence of SEQ ID NO: 92 (predicted sequence of β chain of cysteine-substituted, LVL-modified 3333 TCR-C without N-terminal signal peptide): (w) the amino acid sequences of both of SEQ ID NOs: 89 and 90; (x) the amino acid sequences of both of SEQ ID NOs: 91 and 92: (y) the amino acid sequence of SEQ ID NO: 93 (α chain of 1913 TCR-41BB with N-terminal signal peptide), wherein: (i) X at position 178 of SEQ ID NO: 93 is Thr or Cys: (ii) X at position 242 of SEQ ID NO: 93 is Ser, Ala. Val, Leu. Ile. Pro, Phe. Met, or Trp: (iii) X at position 244 of SEQ ID NO: 93 is Met, Ala. Val, Leu. Ile. Pro, Phe, or Trp; and (iv) X at position 245 of SEQ ID NO: 93 is Gly, Ala. Val, Leu, Ile, Pro, Phe, Met, or Trp: (z) the amino acid sequence of SEQ ID NO: 94 (βchain of 1913 TCR-41BB with N-terminal signal peptide), wherein X at position 190 of SEQ ID NO: 94 is Ser or Cys: (aa) the amino acid sequences of both of SEQ ID NOs: 93 and 94; (bb) the amino acid sequence of SEQ ID NO: 95 (predicted sequence of α chain of 1913 TCR-41BB without N-terminal signal peptide), wherein: (i) X at position 160 of SEQ ID NO: 95 is Thr or Cys: (ii) X at position 224 of SEQ ID NO: 95 is Ser. Ala, Val, Leu, Ile, Pro, Phe, Met, or Trp: (iii) X at position 226 of SEQ ID NO: 95 is Met, Ala. Val, Leu, Ile. Pro, Phe, or Trp; and (iv) X at position 227 of SEQ ID NO: 95 is Gly. Ala. Val. Leu. Ile. Pro. Phe. Met, or Trp: (cc) the amino acid sequence of SEQ ID NO: 96 (predicted sequence of β chain of 1913 TCR-41BB without N-terminal signal peptide), wherein X at position 169 of SEQ ID NO: 96 is Ser or Cys: (dd) the amino acid sequences of both of SEQ ID NOs: 95 and 96; (ee) the amino acid sequence of SEQ ID NO: 97 (α chain of cysteine-substituted, LVL-modified 1913 TCR-41BB with N-terminal signal peptide): (ff) the amino acid sequence of SEQ ID NO: 98 (β chain of cysteine-substituted, LVL-modified 1913 TCR-41BB with N-terminal signal peptide): (gg) the amino acid sequence of SEQ ID NO: 99 (predicted sequence of αchain of cysteine-substituted, LVL-modified 1913 TCR-41BB without N-terminal signal peptide): (hh) the amino acid sequence of SEQ ID NO: 100 (predicted sequence of β chain of cysteine-substituted, LVL-modified 1913 TCR-41BB without N-terminal signal peptide): (ii) the amino acid sequences of both of SEQ ID NOs: 97 and 98: (jj) the amino acid sequences of both of SEQ ID NOs: 99 and 100; (kk) the amino acid sequence of SEQ ID NO: 101 (αchain of 4286 TCR 10-1 with N-terminal signal peptide), wherein: (i) X at position 176 of SEQ ID NO: 101 is Thr or Cys: (ii) X at position 240 of SEQ ID NO: 101 is Ser, Ala, Val, Leu, Ile, Pro, Phe, Met, or Trp: (iii) X at position 242 of SEQ ID NO: 101 is Met, Ala, Val, Leu, Ile, Pro, Phe, or Trp; and (iv) X at position 243 of SEQ ID NO: 101 is Gly, Ala, Val, Leu, Ile, Pro. Phe, Met, or Trp: (11) the amino acid sequence of SEQ ID NO: 102 (β chain of 4286 TCR 10-1 with N-terminal signal peptide), wherein X at position 190 of SEQ ID NO: 102 is Ser or Cys: (mm) the amino acid sequences of both of SEQ ID NOs: 101 and 102: (nn) the amino acid sequence of SEQ ID NO: 103 (predicted sequence of α chain of 4286 TCR 10-1 without N-terminal signal peptide), wherein: (i) X at position 156 of SEQ ID NO: 103 is Thr or Cys: (ii) X at position 220 of SEQ ID NO: 103 is Ser. Ala. Val. Leu. Ile. Pro. Phe. Met, or Trp: (iii) X at position 222 of SEQ ID NO: 103 is Met, Ala. Val, Leu. Ile. Pro, Phe, or Trp; and (iv) X at position 223 of SEQ ID NO: 103 is Gly, Ala. Val, Leu. Ile. Pro, Phe. Met. or Trp: (oo) the amino acid sequence of SEQ ID NO: 104 (predicted sequence of β chain of 4286 TCR 10-1 without N-terminal signal peptide), wherein X at position 169 of SEQ ID NO: 104 is Ser or Cys: (pp) the amino acid sequences of both of SEQ ID NOs: 103 and 104; (qq) the amino acid sequence of SEQ ID NO: 105 (α chain of cysteine-substituted, LVL-modified 4286 TCR 10-1 with N-terminal signal peptide); (rr) the amino acid sequence of SEQ ID NO: 106 (β chain of cysteine-substituted, LVL-modified 4286 TCR 10-1 with N-terminal signal peptide): (ss) the amino acid sequence of SEQ ID NO: 107 (predicted sequence of α chain of cysteine-substituted, LVL-modified 4286 TCR 10-1 without N-terminal signal peptide): (tt) the amino acid sequence of SEQ ID NO: 108 (predicted sequence of β chain of cysteine-substituted, L.VI.-modified 4286 TCR 10-1 without N-terminal signal peptide): (uu) the amino acid sequences of both of SEQ ID NOs: 105 and 106: (vv) the amino acid sequences of both of SEQ ID NOs: 107 and 108: (ww) the amino acid sequence of SEQ ID NO: 109 (α chain of 4286 TCR 10-2 with N-terminal signal peptide), wherein: (i) X at position 180 of SEQ ID NO: 109 is Thr or Cys: (ii) X at position 244 of SEQ ID NO: 109 is Ser, Ala Val, Leu, Ile, Pro, Phe, Met, or Trp; (iii) X at position 246 of SEQ ID NO: 109 is Met, Ala, Val, Leu, Ile, Pro. Phe, or Trp; and (iv) X at position 247 of SEQ ID NO: 109 is Gly, Ala, Val, Leu, Ile, Pro. Phe, Met, or Trp: (xx) the amino acid sequence of SEQ ID NO: 110 (β chain of 4286 TCR 10-2 with N-terminal signal peptide), wherein X at position 192 of SEQ ID NO: 110 is Ser or Cys: (yy) the amino acid sequences of both of SEQ ID NOs: 109 and 110; (zz) the amino acid sequence of SEQ ID NO: 111 (predicted sequence of α chain of 4286 TCR 10-2 without N-terminal signal peptide), wherein: (i) X at position 160 of SEQ ID NO: 111 is Thr or Cys: (ii) X at position 224 of SEQ ID NO: 111 is Ser, Ala. Val, Leu. Ile. Pro, Phe. Met, or Trp: (iii) X at position 226 of SEQ ID NO: 111 is Met, Ala, Val, Leu, Ile, Pro. Phe, or Trp; and (iv) X at position 227 of SEQ ID NO: 111 is Gly, Ala, Val, Leu, Ile, Pro, Phe, Met, or Trp: (aaa) the amino acid sequence of SEQ ID NO: 112 (predicted sequence of β chain of 4286 TCR 10-2 without N-terminal signal peptide), wherein X at position 171 of SEQ ID NO: 112 is Ser or Cys: (bbb) the amino acid sequences of both of SEQ ID NOs: 111 and 112: (ccc) the amino acid sequence of SEQ ID NO: 113 (α chain of cysteine-substituted, LVL-modified 4286 TCR 10-2 with N-terminal signal peptide): (ddd) the amino acid sequence of SEQ ID NO: 114 (βchain of cysteine-substituted. LVL-modified 4286 TCR 10-2 with N-terminal signal peptide): (eee) the amino acid sequence of SEQ ID NO: 115 (predicted sequence of α chain of cysteine-substituted. LVL-modified 4286 TCR 10-2 without N-terminal signal peptide): (fff) the amino acid sequence of SEQ ID NO: 116 (predicted sequence of β chain of cysteine-substituted, LVL-modified 4286 TCR 10-2 without N-terminal signal peptide): (ggg) the amino acid sequences of both of SEQ ID NOs: 113 and 114: (hhh) the amino acid sequences of both of SEQ ID NOs: 115 and 116; (iii) the amino acid sequence of SEQ ID NO: 117 (αchain of 4286 TCR 10-3 with N-terminal signal peptide), wherein: (i) X at position 180 of SEQ ID NO: 117 is Thr or Cys: (ii) X at position 244 of SEQ ID NO: 117 is Ser, Ala, Val, Leu, Ile, Pro, Phe, Met, or Trp: (iii) X at position 246 of SEQ ID NO: 117 is Met, Ala, Val, Leu, Ile, Pro. Phe, or Trp; and (iv) X at position 247 of SEQ ID NO: 117 is Gly, Ala. Val, Leu. Ile. Pro. Phe. Met, or Trp: (jjj) the amino acid sequence of SEQ ID NO: 118 (β chain of 4286 TCR 10-3 with N-terminal signal peptide), wherein X at position 198 of SEQ ID NO: 118 is Ser or Cys: (kkk) the amino acid sequences of both of SEQ ID NOs: 117 and 118; (III) the amino acid sequence of SEQ ID NO: 119 (predicted sequence of α chain of 4286 TCR 10-3 without N-terminal signal peptide), wherein: (i) X at position 160 of SEQ ID NO: 119 is Thr or Cys: (ii) X at position 224 of SEQ ID NO: 119 is Ser, Ala Val, Leu. Ile. Pro, Phe. Met, or Trp: (iii) X at position 226 of SEQ ID NO: 119 is Met, Ala, Val, Leu. Ile. Pro, Phe, or Trp; and (iv) X at position 227 of SEQ ID NO: 119 is Gly, Ala, Val, Leu. Ile. Pro, Phe. Met, or Trp: (mmm) the amino acid sequence of SEQ ID NO: 120 (predicted sequence of β chain of 4286 TCR 10-3 without N-terminal signal peptide), wherein X at position 177 of SEQ ID NO: 120 is Ser or Cys: (nnn) the amino acid sequences of both of SEQ ID NOs: 119 and 120; (000) the amino acid sequence of SEQ ID NO: 121 (α chain of cysteine-substituted, LVL-modified 4286 TCR 10-3 with N-terminal signal peptide): (ppp) the amino acid sequence of SEQ ID NO: 122 (β chain of cysteine-substituted. LVL-modified 4286 TCR 10-3 with N-terminal signal peptide); (qqq) the amino acid sequence of SEQ ID NO: 123 (predicted sequence of α chain of cysteine-substituted, LVL-modified 4286 TCR 10-3 without N-terminal signal peptide); (rrr) the amino acid sequence of SEQ ID NO: 124 (predicted sequence of β chain of cysteine-substituted, LVL-modified 4286 TCR 10-3 without N-terminal signal peptide): (sss) the amino acid sequences of both of SEQ ID NOs: 121 and 122: (ttt) the amino acid sequences of both of SEQ ID NOs: 123 and 124; (uuu) the amino acid sequence of SEQ ID NO: 125 (α chain of 4286 TCR 10-4 with N-terminal signal peptide), wherein: (i) X at position 180 of SEQ ID NO: 125 is Thr or Cys: (ii) X at position 244 of SEQ ID NO: 125 is Ser. Ala. Val. Leu. Ile. Pro. Phe. Met, or Trp: (iii) X at position 246 of SEQ ID NO: 125 is Met, Ala. Val, Leu. Ile. Pro, Phe, or Trp; and (iv) X at position 247 of SEQ ID NO: 125 is Gly, Ala. Val, Leu, Ile, Pro, Phe, Met, or Trp: (vvv) the amino acid sequence of SEQ ID NO: 126 (chain of 4286 TCR 10-4 with N-terminal signal peptide), wherein X at position 198 of SEQ ID NO: 126 is Ser or Cys: (www) the amino acid sequences of both of SEQ ID NOs: 125 and 126: (xxx) the amino acid sequence of SEQ ID NO: 127 (predicted sequence of α chain of 4286 TCR 10-4 without N-terminal signal peptide), wherein: (i) X at position 160 of SEQ ID NO: 127 is Thr or Cys: (ii) X at position 224 of SEQ ID NO: 127 is Ser. Ala, Val, Leu, Ile, Pro, Phe, Met, or Trp: (iii) X at position 226 of SEQ ID NO: 127 is Met, Ala. Val, Leu, Ile, Pro, Phe, or Trp; and (iv) X at position 227 of SEQ ID NO: 127 is Gly, Ala. Val, Leu. Ile, Pro, Phe, Met, or Trp: (yyy) the amino acid sequence of SEQ ID NO: 128 (predicted sequence of β chain of 4286 TCR 10-4 without N-terminal signal peptide), wherein X at position 177 of SEQ ID NO: 128 is Ser or Cys: (zzz) the amino acid sequences of both of SEQ ID NOs: 127 and 128; (aaaa) the amino acid sequence of SEQ ID NO: 129 (α chain of cysteine-substituted, LVL-modified 4286 TCR 10-4 with N-terminal signal peptide): (bbbb) the amino acid sequence of SEQ ID NO: 130 (βchain of cysteine-substituted. LVL-modified 4286 TCR 10-4 with N-terminal signal peptide): (cccc) the amino acid sequence of SEQ ID NO: 131 (predicted sequence of α chain of cysteine-substituted, LVL-modified 4286 TCR 10-4 without N-terminal signal peptide): (dddd) the amino acid sequence of SEQ ID NO: 132 (predicted sequence of β chain of cysteine-substituted. LVL-modified 4286 TCR 10-4 without N-terminal signal peptide): (eeee) the amino acid sequences of both of SEQ ID NOs: 129 and 130; or (ffff) the amino acid sequences of both of SEQ ID NOs: 131 and 132. In an aspect of the invention, one or more of SEQ ID NOs: 77-80, 85-88, 93-96, 101-104, 109-112, 117-120, and 125-128 of the polypeptide are as defined in any one of Tables 1-3.

An aspect of the invention provides a protein comprising at least one of the polypeptides described herein. By “protein” is meant a molecule comprising one or more polypeptide chains.

In an aspect of the invention, the protein of the invention can comprise (a) a first polypeptide chain comprising the amino acid sequences of SEQ ID NOs: 1-3 and a second polypeptide chain comprising the amino acid sequences of SEQ ID NOs: 4-6: (b) a first polypeptide chain comprising the amino acid sequences of SEQ ID NOs: 11-13 and a second polypeptide chain comprising the amino acid sequences of SEQ ID NOs: 14-16: (c) a first polypeptide chain comprising the amino acid sequences of SEQ ID NOs: 21-23 and a second polypeptide chain comprising the amino acid sequences of SEQ ID NOs: 24-26: (d) a first polypeptide chain comprising the amino acid sequences of SEQ ID NOs: 31-33 and a second polypeptide chain comprising the amino acid sequences of SEQ ID NOs: 34-36: (e) a first polypeptide chain comprising the amino acid sequences of SEQ ID NOs: 41-43 and a second polypeptide chain comprising the amino acid sequences of SEQ ID NOs: 44-46; (f) a first polypeptide chain comprising the amino acid sequences of SEQ ID NOs: 51-53 and a second polypeptide chain comprising the amino acid sequences of SEQ ID NOs: 54-56; or (g) a first polypeptide chain comprising the amino acid sequences of SEQ ID NOs: 61-63 and a second polypeptide chain comprising the amino acid sequences of SEQ ID NOs: 64-66.

In another aspect of the invention, (i) the first polypeptide chain comprises the amino acid sequence of SEQ ID NO: 7 and the second polypeptide chain comprises the amino acid sequence of SEQ ID NO: 8: (ii) the first polypeptide chain comprises the amino acid sequence of SEQ ID NO: 9 and the second polypeptide chain comprises the amino acid sequence of SEQ ID NO: 10; (iii) the first polypeptide chain comprises the amino acid sequence of SEQ ID NO: 17 and the second polypeptide chain comprises the amino acid sequence of SEQ ID NO: 18: (iv) the first polypeptide chain comprises the amino acid sequence of SEQ ID NO: 19 and the second polypeptide chain comprises the amino acid sequence of SEQ ID NO: 20: (v) the first polypeptide chain comprises the amino acid sequence of SEQ ID NO: 27 and the second polypeptide chain comprises the amino acid sequence of SEQ ID NO: 28: (vi) the first polypeptide chain comprises the amino acid sequence of SEQ ID NO: 29 and the second polypeptide chain comprises the amino acid sequence of SEQ ID NO: 30: (vii) the first polypeptide chain comprises the amino acid sequence of SEQ ID NO: 37 and the second polypeptide chain comprises the amino acid sequence of SEQ ID NO: 38: (viii) the first polypeptide chain comprises the amino acid sequence of SEQ ID NO: 39 and the second polypeptide chain comprises the amino acid sequence of SEQ ID NO: 40: (ix) the first polypeptide chain comprises the amino acid sequence of SEQ ID NO: 47 and the second polypeptide chain comprises the amino acid sequence of SEQ ID NO: 48: (x) the first polypeptide chain comprises the amino acid sequence of SEQ ID NO: 49 and the second polypeptide chain comprises the amino acid sequence of SEQ ID NO: 50: (xi) the first polypeptide chain comprises the amino acid sequence of SEQ ID NO: 57 and the second polypeptide chain comprises the amino acid sequence of SEQ ID NO: 58: (xii) the first polypeptide chain comprises the amino acid sequence of SEQ ID NO: 59 and the second polypeptide chain comprises the amino acid sequence of SEQ ID NO: 60; (xiii) the first polypeptide chain comprises the amino acid sequence of SEQ ID NO: 67 and the second polypeptide chain comprises the amino acid sequence of SEQ ID NO: 68: or (xiv) the first polypeptide chain comprises the amino acid sequence of SEQ ID NO: 69 and the second polypeptide chain comprises the amino acid sequence of SEQ ID NO: 70.

The inventive protein may further comprise any of the constant regions described herein with respect to other aspects of the invention. In this regard, in an aspect of the invention, (i) the first polypeptide chain may further comprise the amino acid sequence of SEQ ID NO: 71 and the second polypeptide chain may further comprise the amino acid sequence of SEQ ID NO: 72: (ii) the first polypeptide chain may further comprise the amino acid sequence of SEQ ID NO: 73 and the second polypeptide chain may further comprise the amino acid sequence of SEQ ID NO: 74: or (ii) the first polypeptide chain may comprise the amino acid sequence of SEQ ID NO: 75 and the second polypeptide chain may comprise the amino acid sequence of SEQ ID NO: 76. In an aspect of the invention, one or both of SEQ ID NOs: 71 and 72 of the protein are as defined in any one of Tables 1-3.

The inventive protein may comprise a full length a or β chain, as described herein with respect to other aspects of the invention. In this regard, in an aspect of the invention, (1) the first polypeptide chain comprises the amino acid sequence of SEQ ID NO: 77, wherein: (i) X at position 182 of SEQ ID NO: 77 is Thr or Cys: (ii) X at position 246 of SEQ ID NO: 77 is Ser. Ala. Val. Leu. Ile. Pro. Phe. Met, or Trp: (iii) X at position 248 of SEQ ID NO: 77 is Met, Ala. Val, Leu, Ile, Pro, Phe, or Trp; and (iv) X at position 249 of SEQ ID NO: 77 is Gly, Ala, Val, Leu, Ile. Pro, Phe. Met, or Trp: (2) the second polypeptide chain comprises the amino acid sequence of SEQ ID NO: 78, wherein X at position 188 of SEQ ID NO: 78 is Ser or Cys: (3) both (1) and (2): (4) the first polypeptide chain comprises the amino acid sequence of SEQ ID NO: 79, wherein: (i) X at position 161 of SEQ ID NO: 79 is Thr or Cys: (ii) X at position 225 of SEQ ID NO: 79 is Ser, Ala, Val, Leu. Ile, Pro, Phe, Met, or Trp: (iii) X at position 227 of SEQ ID NO: 79 is Met, Ala, Val, Leu, Ile, Pro. Phe, or Trp; and (iv) X at position 228 of SEQ ID NO: 79 is Gly, Ala. Val, Leu. Ile. Pro, Phe. Met, or Trp: (5) the second polypeptide chain comprises the amino acid sequence of SEQ ID NO: 80, wherein X at position 166 of SEQ ID NO: 80 is Ser or Cys: (6) both (4) and (5): (7) SEQ ID NO: 81: (8) SEQ ID NO: 82: (9) SEQ ID NO: 83: (10) SEQ ID NO: 84: (11) both (7) and (8): (12) both (9) and (10): (13) the first polypeptide chain comprises the amino acid sequence of SEQ ID NO: 85, wherein: (i) X at position 184 of SEQ ID NO: 85 is Thr or Cys: (ii) X at position 248 of SEQ ID NO: 85 is Ser, Ala, Val, Leu, Ile, Pro, Phe, Met, or Trp: (iii) X at position 250 of SEQ ID NO: 85 is Met, Ala. Val, Leu. Ile. Pro, Phe, or Trp; and (iv) X at position 251 of SEQ ID NO: 85 is Gly. Ala, Val. Leu, Ile, Pro. Phe, Met, or Trp: (14) the second polypeptide chain comprises the amino acid sequence of SEQ ID NO: 86, wherein X at position 188 of SEQ ID NO: 86 is Ser or Cys: (15) both (13) and (14): (16) the first polypeptide chain comprises the amino acid sequence of SEQ ID NO: 87, wherein: (i) X at position 163 of SEQ ID NO: 87 is Thr or Cys: (ii) X at position 227 of SEQ ID NO: 87 is Ser, Ala. Val, Leu. Ile. Pro. Phe, Met, or Trp: (iii) X at position 229 of SEQ ID NO: 87 is Met, Ala, Val, Leu, Ile, Pro, Phe, or Trp; and (iv) X at position 230 of SEQ ID NO: 87 is Gly, Ala, Val, Leu, Ile, Pro, Phe, Met, or Trp: (17) the second polypeptide chain comprises the amino acid sequence of SEQ ID NO: 88, wherein X at position 167 of SEQ ID NO: 88 is Ser or Cys: (18) both (16) and (17): (19) SEQ ID NO: 89: (20) SEQ ID NO: 90; (21) SEQ ID NO: 91: (22) SEQ ID NO: 92: (23) both (19) and (20): (24) both (21) and (22): (25) the first polypeptide chain comprises the amino acid sequence of SEQ ID NO: 93, wherein: (i) X at position 178 of SEQ ID NO: 93 is Thr or Cys: (ii) X at position 242 of SEQ ID NO: 93 is Ser. Ala, Val. Leu, Ile, Pro. Phe, Met, or Trp: (iii) X at position 244 of SEQ ID NO: 93 is Met, Ala. Val, Leu, Ile, Pro, Phe, or Trp; and (iv) X at position 245 of SEQ ID NO: 93 is Gly. Ala, Val. Leu, Ile, Pro, Phe, Met, or Trp: (26) the second polypeptide chain comprises the amino acid sequence of SEQ ID NO: 94, wherein X at position 190 of SEQ ID NO: 94 is Ser or Cys: (27) both (25) and (26): (28) the first polypeptide chain comprises the amino acid sequence of SEQ ID NO: 95, wherein: (i) X at position 160 of SEQ ID NO: 95 is Thr or Cys: (ii) X at position 224 of SEQ ID NO: 95 is Ser, Ala, Val, Leu, Ile, Pro. Phe, Met, or Trp: (iii) X at position 226 of SEQ ID NO: 95 is Met, Ala, Val, Leu. Ile. Pro, Phe, or Trp; and (iv) X at position 227 of SEQ ID NO: 95 is Gly, Ala. Val, Leu. Ile. Pro, Phe. Met, or Trp: (29) the second polypeptide chain comprises the amino acid sequence of SEQ ID NO: 96, wherein X at position 169 of SEQ ID NO: 96 is Ser or Cys: (30) both (28) and (29): (31) SEQ ID NO: 97: (32) SEQ ID NO: 98: (33) SEQ ID NO: 99: (34) SEQ ID NO: 100: (35) both (31) and (32): (36) both (33) and (34): (37) the first polypeptide chain comprises the amino acid sequence of SEQ ID NO: 101, wherein: (i) X at position 176 of SEQ ID NO: 101 is Thr or Cys: (ii) X at position 240 of SEQ ID NO: 101 is Ser, Ala Val, Leu. Ile, Pro, Phe, Met, or Trp; (iii) X at position 242 of SEQ ID NO: 101 is Met, Ala, Val, Leu, Ile, Pro. Phe, or Trp; and (iv) X at position 243 of SEQ ID NO: 101 is Gly, Ala, Val, Leu, Ile, Pro. Phe, Met, or Trp: (38) the second polypeptide chain comprises the amino acid sequence of SEQ ID NO: 102, wherein X at position 190 of SEQ ID NO: 102 is Ser or Cys: (39) both (37) and (38): (40) the first polypeptide chain comprises the amino acid sequence of SEQ ID NO: 103, wherein: (i) X at position 156 of SEQ ID NO: 103 is Thr or Cys: (ii) X at position 220 of SEQ ID NO: 103 is Ser. Ala, Val. Leu, Ile, Pro, Phe, Met, or Trp: (iii) X at position 222 of SEQ ID NO: 103 is Met, Ala. Val, Leu, Ile, Pro, Phe, or Trp; and (iv) X at position 223 of SEQ ID NO: 103 is Gly, Ala. Val, Leu. Ile. Pro, Phe. Met, or Trp: (41) the second polypeptide chain comprises the amino acid sequence of SEQ ID NO: 104, wherein X at position 169 of SEQ ID NO: 104 is Ser or Cys: (42) both (40) and (41): (43) SEQ ID NO: 105: (44) SEQ ID NO: 106; (45) SEQ ID NO: 107: (46) SEQ ID NO: 108: (47) both (43) and (44): (48) both (45) and (46): (49) the first polypeptide chain comprises the amino acid sequence of SEQ ID NO: 109, wherein: (i) X at position 180 of SEQ ID NO: 109 is Thr or Cys: (ii) X at position 244 of SEQ ID NO: 109 is Ser, Ala Val, Leu. Ile. Pro, Phe. Met, or Trp: (iii) X at position 246 of SEQ ID NO: 109 is Met, Ala, Val, Leu, Ile, Pro. Phe, or Trp; and (iv) X at position 247 of SEQ ID NO: 109 is Gly, Ala, Val, Leu, Ile, Pro, Phe, Met, or Trp: (50) the second polypeptide chain comprises the amino acid sequence of SEQ ID NO: 110, wherein X at position 192 of SEQ ID NO: 110 is Ser or Cys: (51) both (49) and (50): (52) the first polypeptide chain comprises the amino acid sequence of SEQ ID NO: 111, wherein: (i) X at position 160 of SEQ ID NO: 111 is Thr or Cys: (ii) X at position 224 of SEQ ID NO: 111 is Ser, Ala, Val, Leu, Ile, Pro, Phe. Met, or Trp: (iii) X at position 226 of SEQ ID NO: 111 is Met. Ala. Val. Leu. Ile. Pro. Phe, or Trp; and (iv) X at position 227 of SEQ ID NO: 111 is Gly, Ala, Val, Leu, Ile. Pro, Phe. Met, or Trp: (53) the second polypeptide chain comprises the amino acid sequence of SEQ ID NO: 112, wherein X at position 171 of SEQ ID NO: 112 is Ser or Cys: (54) both (52) and (53): (55) SEQ ID NO: 113: (56) SEQ ID NO: 114: (57) SEQ ID NO: 115: (58) SEQ ID NO: 116: (59) both (55) and (56): (60) both (57) and (58): (61) the first polypeptide chain comprises the amino acid sequence of SEQ ID NO: 117, wherein: (i) X at position 180 of SEQ ID NO: 117 is Thr or Cys: (ii) X at position 244 of SEQ ID NO: 117 is Ser, Ala. Val, Leu, Ile, Pro, Phe, Met, or Trp: (iii) X at position 246 of SEQ ID NO: 117 is Met. Ala, Val. Leu, Ile, Pro. Phe, or Trp; and (iv) X at position 247 of SEQ ID NO: 117 is Gly, Ala. Val, Leu. Ile. Pro, Phe, Met, or Trp: (62) the second polypeptide chain comprises the amino acid sequence of SEQ ID NO: 118, wherein X at position 198 of SEQ ID NO: 118 is Ser or Cys: (63) both (61) and (62): (64) the first polypeptide chain comprises the amino acid sequence of SEQ ID NO: 119, wherein: (i) X at position 160 of SEQ ID NO: 119 is Thr or Cys: (ii) X at position 224 of SEQ ID NO: 119 is Ser. Ala, Val. Leu, Ile, Pro. Phe, Met, or Trp: (iii) X at position 226 of SEQ ID NO: 119 is Met, Ala. Val, Leu, Ile, Pro, Phe, or Trp; and (iv) X at position 227 of SEQ ID NO: 119 is Gly, Ala, Val, Leu, Ile. Pro, Phe. Met, or Trp: (65) the second polypeptide chain comprises the amino acid sequence of SEQ ID NO: 120, wherein X at position 177 of SEQ ID NO: 120 is Ser or Cys: (66) both (64) and (65): (67) SEQ ID NO: 121: (68) SEQ ID NO: 122: (69) SEQ ID NO: 123: (70) SEQ ID NO: 124: (71) both (67) and (68): (72) both (69) and (70): (73) the first polypeptide chain comprises the amino acid sequence of SEQ ID NO: 125, wherein: (i) X at position 180 of SEQ ID NO: 125 is Thr or Cys: (ii) X at position 244 of SEQ ID NO: 125 is Ser, Ala. Val, Leu, Ile, Pro, Phe, Met, or Trp: (iii) X at position 246 of SEQ ID NO: 125 is Met. Ala, Val. Leu, Ile, Pro. Phe, or Trp; and (iv) X at position 247 of SEQ ID NO: 125 is Gly. Ala. Val. Leu. Ile. Pro. Phe. Met, or Trp: (74) the second polypeptide chain comprises the amino acid sequence of SEQ ID NO: 126, wherein X at position 198 of SEQ ID NO: 126 is Ser or Cys: (75) both (73) and (74): (76) the first polypeptide chain comprises the amino acid sequence of SEQ ID NO: 127, wherein: (i) X at position 160 of SEQ ID NO: 127 is Thr or Cys: (ii) X at position 224 of SEQ ID NO: 127 is Ser. Ala, Val. Leu, Ile, Pro. Phe, Met, or Trp: (iii) X at position 226 of SEQ ID NO: 127 is Met, Ala, Val, Leu. Ile. Pro, Phe, or Trp; and (iv) X at position 227 of SEQ ID NO: 127 is Gly, Ala, Val, Leu. Ile. Pro, Phe. Met, or Trp: (77) the second polypeptide chain comprises the amino acid sequence of SEQ ID NO: 128, wherein X at position 177 of SEQ ID NO: 128 is Ser or Cys: (78) both (76) and (77): (79) SEQ ID NO: 129: (80) SEQ ID NO: 130: (81) SEQ ID NO: 131: (82) SEQ ID NO: 132: (83) both (79) and (80): or (84) both (81) and (82). In an aspect of the invention, one or more of SEQ ID NOs: 77-80, 85-88, 93-96, 101-104, 109-112, 117-120, and 125-128 of the protein are as defined in any one of Tables 1-3.

The protein of the invention can be a TCR. Alternatively, if, for example, the protein comprises a single polypeptide chain comprising the amino acid sequences of both the TCR α and β chains, or if the first and/or second polypeptide chain(s) of the protein further comprise(s) other amino acid sequences, e.g., an amino acid sequence encoding an immunoglobulin or a portion thereof, then the inventive protein can be a fusion protein. In this regard, the invention also provides a fusion protein comprising at least one of the inventive polypeptides described herein along with at least one other polypeptide. The other polypeptide can exist as a separate polypeptide of the fusion protein, or can exist as a polypeptide, which is expressed in frame (in tandem) with one of the inventive polypeptides described herein. The other polypeptide can encode any peptidic or proteinaceous molecule, or a portion thereof, including, but not limited to an immunoglobulin. CD3. CD4, CD8, an MHC molecule, a CD1 molecule, e.g., CD1a, CD1b, CD1c, CD1d, etc.

The fusion protein can comprise one or more copies of the inventive polypeptide and/or one or more copies of the other polypeptide. For instance, the fusion protein can comprise 1, 2, 3, 4, 5, or more, copies of the inventive polypeptide and/or of the other polypeptide. Suitable methods of making fusion proteins are known in the art, and include, for example, recombinant methods.

In some aspects of the invention, the TCRs, polypeptides, and proteins of the invention may be expressed as a single protein comprising a linker peptide linking the α chain and the βchain. In this regard, the TCRs, polypeptides, and proteins of the invention may further comprise a linker peptide. The linker peptide may advantageously facilitate the expression of a recombinant TCR, polypeptide, and/or protein in a host cell. The linker peptide may comprise any suitable amino acid sequence. The linker peptide may be a cleavable linker peptide. For example, the linker peptide may be a furin-SGSG-P2A linker peptide comprising the amino acid sequence of RAKRSGSGATNFSLLKQAGDVEENPGP (SEQ ID NO: 133) or RAKRSGSGATNFSLLKQAGDVEENPG (SEQ ID NO: 146). Upon expression of the construct including the linker peptide by a host cell, the linker peptide may be cleaved, resulting in separated α and β chains. In an aspect of the invention, the TCR, polypeptide, or protein may comprise an amino acid sequence comprising a full-length a chain, a full-length β chain, and a linker peptide positioned between the α and β chains.

The protein of the invention can be a recombinant antibody, or an antigen binding portion thereof, comprising at least one of the inventive polypeptides described herein. As used herein, “recombinant antibody” refers to a recombinant (e.g., genetically engineered) protein comprising at least one of the polypeptides of the invention and a polypeptide chain of an antibody, or an antigen binding portion thereof. The polypeptide of an antibody, or antigen binding portion thereof, can be a heavy chain, a light chain, a variable or constant region of a heavy or light chain, a single chain variable fragment (scFv), or an Fc, Fab, or F(ab)₂′ fragment of an antibody, etc. The polypeptide chain of an antibody, or an antigen binding portion thereof, can exist as a separate polypeptide of the recombinant antibody. Alternatively, the polypeptide chain of an antibody, or an antigen binding portion thereof, can exist as a polypeptide, which is expressed in frame (in tandem) with the polypeptide of the invention. The polypeptide of an antibody, or an antigen binding portion thereof, can be a polypeptide of any antibody or any antibody fragment, including any of the antibodies and antibody fragments described herein.

Included in the scope of the invention are functional variants of the inventive TCRs, polypeptides, or proteins described herein. The term “functional variant,” as used herein, refers to a TCR, polypeptide, or protein having substantial or significant sequence identity or similarity to a parent TCR, polypeptide, or protein, which functional variant retains the biological activity of the TCR, polypeptide, or protein of which it is a variant. Functional variants encompass, for example, those variants of the TCR, polypeptide, or protein described herein (the parent TCR, polypeptide, or protein) that retain the ability to specifically bind to the mutated CDKN2A peptide for which the parent TCR has antigenic specificity or to which the parent polypeptide or protein specifically binds, to a similar extent, the same extent, or to a higher extent, as the parent TCR, polypeptide, or protein. In reference to the parent TCR, polypeptide, or protein, the functional variant can, for instance, be at least about 30%, about 50%, about 75%, about 80%, about 90%, about 95%, about 96%, about 97%, about 98%, about 99% or more identical in amino acid sequence to the parent TCR, polypeptide, or protein, respectively.

The functional variant can, for example, comprise the amino acid sequence of the parent TCR, polypeptide, or protein with at least one conservative amino acid substitution. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

Alternatively or additionally, the functional variants can comprise the amino acid sequence of the parent TCR, polypeptide, or protein with at least one non-conservative amino acid substitution. In this case, it is preferable for the non-conservative amino acid substitution to not interfere with or inhibit the biological activity of the functional variant. Preferably, the non-conservative amino acid substitution enhances the biological activity of the functional variant, such that the biological activity of the functional variant is increased as compared to the parent TCR, polypeptide, or protein.

The TCR, polypeptide, or protein can consist essentially of the specified amino acid sequence or sequences described herein, such that other components of the TCR, polypeptide, or protein, e.g., other amino acids, do not materially change the biological activity of the TCR, polypeptide, or protein. In this regard, the inventive TCR, polypeptide, or protein can, for example, consist essentially of the amino acid sequence of SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88. SEQ ID NO: 89. SEQ ID NO: 90. SEQ ID NO: 91. SEQ ID NO: 92. SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107. SEQ ID NO: 108, SEQ ID NO: 109, SEQ ID NO: 110. SEQ ID NO: 111, SEQ ID NO: 112. SEQ ID NO: 113, SEQ ID NO: 114. SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122. SEQ ID NO: 123, SEQ ID NO: 124, SEQ ID NO: 125, SEQ ID NO: 126, SEQ ID NO: 127. SEQ ID NO: 128, SEQ ID NO: 129, SEQ ID NO: 130. SEQ ID NO: 131, or SEQ ID NO: 132, both of SEQ ID NOs: 77-78, both of SEQ ID NOs: 79-80, both of SEQ ID NOs: 81-82, both of SEQ ID NOS: 83-84, both of SEQ ID NOs: 85-86, both of SEQ ID NOs: 87-88, both of SEQ ID NOs: 89-90, both of SEQ ID NOs: 91-92, both of SEQ ID NOs: 93-94, both of SEQ ID NOs: 95-96, both of SEQ ID NOs: 97-98, both of SEQ ID NOs: 99-100, both of SEQ ID NOs: 101-102, both of SEQ ID NOs: 103-104, both of SEQ ID NOs: 105-106, both of SEQ ID NOs: 107-108, both of SEQ ID NOs: 109-110, both of SEQ ID NOs: 111-112, both of SEQ ID NOS: 113-114, both of SEQ ID NOs: 115-116, both of SEQ ID NOs: 117-118, both of SEQ ID NOs: 119-120, both of SEQ ID NOs: 121-122, both of SEQ ID NOs: 123-124, both of SEQ ID NOs: 125-126, both of SEQ ID NOs: 127-128, both of SEQ ID NOs: 129-130, or both of SEQ ID NOs: 131-132.

Also, for instance, the inventive TCRs, polypeptides, or proteins can consist essentially of the amino acid sequence(s) of (1) SEQ ID NO: 7, (2) SEQ ID NO: 8, (3) SEQ ID NO: 9, (4) SEQ ID NO: 10. (5) SEQ ID NO: 17, (6) SEQ ID NO: 18, (7) SEQ ID NO: 19, (8) SEQ ID NO: 20. (9) SEQ ID NO: 27. (10) SEQ ID NO: 28. (11) SEQ ID NO: 29. (12) SEQ ID NO: 30, (13) SEQ ID NO: 37. (14) SEQ ID NO: 38, (15) SEQ ID NO: 39, (16) SEQ ID NO: 40. (17) SEQ ID NO: 47, (18) SEQ ID NO: 48. (19) SEQ ID NO: 49, (20) SEQ ID NO: 50. (21) SEQ ID NO: 57, (22) SEQ ID NO: 58. (23) SEQ ID NO: 59, (24) SEQ ID NO: 60, (25) SEQ ID NO: 67. (26) SEQ ID NO: 68, (27) SEQ ID NO: 69, (28) SEQ ID NO: 70, (29) both of SEQ ID NOs: 7 and 8, (30) both of SEQ ID NOs: 9 and 10, (31) both of SEQ ID NOs: 17 and 18, (32) both of SEQ ID NOs: 19 and 20. (33) both of SEQ ID NOs: 27 and 28, (34) both of SEQ ID NOs: 29 and 30. (35) both of SEQ ID NOs: 37 and 38. (36) both of SEQ ID NOs: 39 and 40, (37) both of SEQ ID NOs: 47 and 48, (38) both of SEQ ID NOs: 49 and 50, (39) both of SEQ ID NOs: 57 and 58, (40) both of SEQ ID NOs: 59 and 60, (41) both of SEQ ID NOs: 67 and 68, or (42) both of SEQ ID NOs: 69 and 70.

Furthermore, the inventive TCRs, polypeptides, or proteins can consist essentially of the amino acid sequences of (a) all of SEQ ID NOs: 1-3, (b) all of SEQ ID NOs: 4-6, (c) all of SEQ ID NOs: 1-6, (d) all of SEQ ID NOs: 11-13, (e) all of SEQ ID NOs: 14-16, (f) all of SEQ ID NOs: 11-16, (g) all of SEQ ID NOs: 21-23, (h) all of SEQ ID NOs: 24-26, (i) all of SEQ ID NOs: 21-26, (j) all of SEQ ID NOs: 31-33. (k) all of SEQ ID NOs: 34-36. (1) all of SEQ ID NOs: 31-36, (m) all of SEQ ID NOs: 41-43. (n) all of SEQ ID NOs: 44-46. (o) all of SEQ ID NOs: 41-46, (p) all of SEQ ID NOs: 51-53, (q) all of SEQ ID NOs: 54-56, (r) all of SEQ ID NOs: 51-56, (s) all of SEQ ID NOs: 61-63, (t) all of SEQ ID NOs: 64-66, or (u) all of SEQ ID NOS: 61-66.

The TCRs, polypeptides, and proteins of the invention can be of anylength, i.e., can comprise any number of amino acids, provided that the TCRs, polypeptides, or proteins retain their biological activity, e.g., the ability to specifically bind to mutated CDKN2A peptide: detect cancer in a mammal: or treat or prevent cancer in a mammal, etc. For example, the polypeptide can be in the range of from about 50 to about 5000 amino acids long, such as about 50, about 70, about 75, about 100, about 125, about 150, about 175, about 200, about 300, about 400, about 500, about 600, about 700, about 800, about 900, about 1000 or more amino acids in length. In this regard, the polypeptides of the invention also include oligopeptides.

The TCRs, polypeptides, and proteins of the invention can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine. S-acetylaminomethyl-cysteine, trans-3-and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,α-diaminopropionic acid, homophenylalanine, and α-tert-butylglycine.

The TCRs, polypeptides, and proteins of the invention can be glycosylated, amidated, carboxylated, phosphorylated, esterified, N-acylated, cyclized via, e.g., a disulfide bridge, or converted into an acid addition salt and/or optionally dimerized or polymerized, or conjugated.

The inventive TCRs, polypeptides, and proteins described herein (including any of the functional portions or variants thereof) are also contemplated to be useful as the soluble TCR component of bispecific engager TCR fusion proteins (e.g., IMMTAC (immune-mobilizing monoclonal TCRs against cancer) molecules). Bispecific engager TCR fusion proteins have two components. One component comprises a soluble TCR. The other component comprises an anti-CD3 effector. The anti-CD3 effector may be any molecule that engages with a CD3 molecule on T cells and activates a T cell immune response. For example, the anti-CD3 effector may be an anti-CD3 antibody or anti-CD3 antibody fragment. The soluble TCR component of the bispecific engager TCR fusion protein binds to the target antigen presented on the surface of cancer cells presented by an HLA molecule. The anti-CD3 effector component engages a CD3 molecule on T cells. The engagement of these components of the bispecific engager TCR fusion protein triggers the activation and recruitment of T cells and redirects T-cell killing to tumor cells. An aspect of the invention provides a bispecific engager TCR fusion protein comprising (i) any of the inventive TCRs, polypeptides, or proteins (including any of the functional portions or variants thereof) described herein and (ii) an anti-CD3 engager. Hereinafter, references to “protein(s)” also encompass the bispecific engager TCR fusion proteins described herein, unless specified otherwise.

The TCR, polypeptide, and/or protein of the invention can be obtained by methods known in the art such as, for example, de novo synthesis. Also, polypeptides and proteins can be recombinantly produced using the nucleic acids described herein using standard recombinant methods. See, for instance. Green and Sambrook, Molecular Cloning: A Laboratory Manual, 4^{th} ed., Cold Spring Harbor Press, Cold Spring Harbor. NY (2012). Alternatively, the TCRs, polypeptides, and/or proteins described herein can be synthesized by any of a variety of commercial entities. In this respect, the inventive TCRs, polypeptides, and proteins can be synthetic, recombinant, isolated, and/or purified. An aspect of the invention provides an isolated or purified TCR, polypeptide, protein, or bispecific engager TCR fusion protein encoded by any of the nucleic acids or vectors described herein with respect to other aspects of the invention. Another aspect of the invention provides an isolated or purified TCR, polypeptide, protein, or bispecific engager TCR fusion protein that results from expression of any of the nucleic acids or vectors described herein in a cell. Still another aspect of the invention provides a method of producing any of the TCRs, polypeptides. proteins, or bispecific engager TCR fusion proteins described herein, the method comprising culturing any of the host cells or populations of host cells described herein so that the TCR, polypeptide, protein, or bispecific engager TCR fusion protein is produced.

Included in the scope of the invention are conjugates, e.g., bioconjugates. comprising any of the inventive TCRs, polypeptides, or proteins (including any of the functional portions or variants thereof), nucleic acids, recombinant expression vectors, host cells, or populations of host cells. Conjugates, as well as methods of synthesizing conjugates in general, are known in the art.

An aspect of the invention provides a nucleic acid comprising a nucleotide sequence encoding any of the TCRs, polypeptides, proteins, or bispecific engager TCR fusion proteins described herein. “Nucleic acid,” as used herein, includes “polynucleotide,” “oligonucleotide.” and “nucleic acid molecule,” and generally means a polymer of DNA or RNA, which can be single-stranded or double-stranded, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In an aspect, the nucleic acid comprises complementary DNA (cDNA). It is generally preferred that the nucleic acid does not comprise any insertions, deletions. inversions, and/or substitutions. However, it may be suitable in some instances, as discussed herein, for the nucleic acid to comprise one or more insertions, deletions, inversions, and/or substitutions.

Preferably, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. See, for example, Green and Sambrook et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxy methylaminomethyl-2-thiouridine, 5-carboxy methylaminomethyluracil, dihydrouracil. α-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxy aminomethyl-2-thiouracil, α-D-mannosylqueosine, 5′-methoxy carboxy methyluracil, 5-methoxyuracil, 2-methylthio-N⁶-isopentenyladenine, uracil-5-oxyacetic acid (v), wy butoxosine, pseudouracil. queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be synthesized by any of a variety of commercial entities.

The nucleic acid can comprise any nucleotide sequence which encodes any of the TCRs, polypeptides, or proteins described herein. In an aspect of the invention, the nucleic acid comprises a codon-optimized nucleotide sequence encoding any of the TCRs, polypeptides, or proteins described herein. Without being bound to any particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid. but can be translated by tRNA that is more readily available within a cell, thus increasing translation efficiency. Optimization of the nucleotide sequence may also reduce secondary mRNA structures that would interfere with translation, thus increasing translation efficiency.

An aspect of the invention also provides a nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive TCRs. It is generally appreciated that conditions can be rendered more stringent by the addition of increasing amounts of formamide.

An aspect of the invention provides an isolated or purified nucleic acid comprising, from 5′ to 3″, a first nucleic acid sequence and a second nucleotide sequence, wherein the first and second nucleotide sequence, respectively, encode the amino sequences of SEQ ID NOs: 7 and 8; 8 and 7; 9 and 10; 10 and 9; 17 and 18; 18 and 17; 19 and 20; 20 and 19; 27 and 28; 28 and 27; 29 and 30; 30 and 29; 37 and 38; 38 and 37; 39 and 40; 40 and 39; 47 and 48; 48 and 47; 49 and 50; 50 and 49; 57 and 58; 58 and 57; 59 and 60; 60 and 59; 67 and 68; 68 and 67; 69 and 70; 70 and 69; 77 and 78; 78 and 77; 79 and 80; 80 and 79; 81 and 82; 82 and 81; 83 and 84; 84 and 83; 85 and 86; 86 and 85; 87 and 88; 88 and 87; 89 and 90; 90 and 89; 91 and 92; 92 and 91; 93 and 94; 94 and 93; 95 and 96; 96 and 95; 97 and 98; 98 and 97; 99 and 100; 100 and 99; 101 and 102; 102 and 101; 103 and 104; 104 and 103; 105 and 106; 106 and 105; 107 and 108; 108 and 107; 109 and 110; 111 and 112; 112 and 111; 113 and 114; 114 and 113; 115 and 116; 116 and 115; 117 and 118; 118 and 117; 119 and 120; 120 and 119; 121 and 122; 122 and 121; 123 and 124; 124 and 123; 125 and 126; 126 and 125; 127 and 128; 128 and 127; 129 and 130; 130 and 129; 131 and 132; or 132 and 131.

In an aspect of the invention, the isolated or purified nucleic acid further comprises a third nucleotide sequence interposed between the first and second nucleotide sequence, wherein the third nucleotide sequence encodes a cleavable linker peptide. In an aspect of the invention, the cleavable linker peptide comprises the amino acid sequence of RAKRSGSGATNFSLLKQAGDVEENPGP (SEQ ID NO: 133) or RAKRSGSGATNFSLLKQAGDVEENPG (SEQ ID NO: 146).

The nucleic acids of the invention can be incorporated into a recombinant expression vector. In this regard, the invention provides a recombinant expression vector comprising any of the nucleic acids of the invention. In an aspect of the invention, the recombinant expression vector comprises a nucleotide sequence encoding the a chain, the β chain, and linker peptide.

For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotide, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages do not hinder the transcription or replication of the vector.

The recombinant expression vector of the invention can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences), the pBluescript series (Stratagene. LaJolla. CA). the pET series (Novagen. Madison, WI), the pGEX series (Pharmacia Biotech, Uppsala, Sweden), and the pEX series (Clontech. Palo Alto, CA). Bacteriophage vectors, such as λGT10, λGT11, λZapII (Stratagene), λEMBL4, and λNM1149, also can be used. Examples of plant expression vectors include pBIO1, pBI101.2, pBI101.3, pBI121 and pBIN19 (Clontech). Examples of animal expression vectors include pEUK-Cl, pMAM and pMAMneo (Clontech). Preferably, the recombinant expression vector is a viral vector, e.g., a retroviral vector. In an especially preferred aspect, the recombinant expression vector is an MSGVI vector. In an aspect of the invention, the recombinant expression vector is a transposon or a lentiviral vector.

The recombinant expression vectors of the invention can be prepared using standard recombinant DNA techniques described in, for example. Green and Sambrook et al . . . supra. Constructs of expression vectors, which are circular or linear, can be prepared to contain a replication system functional in a prokaryotic or eukaryotic host cell. Replication systems can be derived, e.g., from ColEl, 2 μ plasmid, λ, SV40, bovine papillomavirus, and the like.

Desirably, the recombinant expression vector comprises regulatory sequences. such as transcription and translation initiation and termination codons, which are specific to the type of host cell (e.g., bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected host cells. Marker genes include biocide resistance, e.g., resistance to antibiotics, heavy metals, etc., complementation in an auxotrophic host cell to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operablylinked to the nucleotide sequence encoding the TCR, polypeptide, or protein, or to the nucleotide sequence which is complementary to or which hybridizes to the nucleotide sequence encoding the TCR, polypeptide, or protein. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, nitroreductase, and the inducible caspase 9 gene system.

Another aspect of the invention further provides a host cell comprising any of the nucleic acids or recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DHSα E, coli cells, Chinese hamster ovarian cells, monkey VERO cells, COS cells, HEK293 cells, and the like. For purposes of amplifying or replicating the recombinant expression vector, the host cell is preferably a prokaryotic cell. e.g., a DH5a cell. For purposes of producing a recombinant TCR, polypeptide, or protein, the host cell is preferably a mammalian cell. Most preferably, the host cell is a human cell. While the host cell can be of any cell type, can originate from any type of tissue, and can be of any developmental stage, the host cell preferably is a peripheral blood lymphocyte (PBL) or a peripheral blood mononuclear cell (PBMC). More preferably, the host cell is a T cell. In an aspect of the invention, the host cell is a human lymphocyte. In another aspect of the invention, the host cell is selected from the group consisting of a T cell, a natural killer T (NKT) cell, an invariant natural killer T (INKT) cell, a natural killer (NK) cell, a macrophage, a pluripotent cell, and a multipotent cell. Still another aspect of the invention provides a method of producing a host cell expressing a TCR that has antigenic specificity for the peptide of AVCPWTWLR (SEQ ID NO: 141), LLVDLAEEL (SEQ ID NO: 142), or RLLVDLAEEL (SEQ ID NO: 143), the method comprising contacting a cell with any of the vectors described herein under conditions that allow introduction of the vector into the cell. For purposes herein, the T cell can be any T cell, such as a cultured T cell, e.g., a primary T cell, or a T cell from a cultured T cell line, e.g., Jurkat. SupTI, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. Preferably, the T cell is a human T cell. The T cell can be any type of T cell and can be of any developmental stage, including but not limited to CD4+/CD8+double positive T cells. CD4+ helper T cells, e.g., Thi and The cells, CD4+ T cells, CD8+ T cells (e.g., cytotoxic T cells), tumor infiltrating lymphocytes (TILs), memory T cells (e.g., central memory T cells and effector memory T cells), naïve T cells, and the like.

In an aspect of the invention, the host cell is a pluripotent cell or a multipotent cell. Pluripotent cells have the capacity to give rise to any of the three germ layers; endoderm, mesoderm, and ectoderm. Pluripotent cells may comprise, for example, stem cells, e.g., embryonic stem cells, nuclear transfer derived embryonic stem cells, induced pluripotent stem cells (iPSC), etc. Multipotent cells may comprise, for example, hematopoietic stem cells. Modifying, e.g., reprogramming, cells to a pluripotent state refers to the reversion of a cell to a pluripotent cell and is described for example, in Crompton et al., Trends Immunol., 35 (4); 178-185 (2014). Exemplary techniques may include somatic cell nuclear transfer (SCNT), cell-cell fusion, and direct reprogramming. Examples of methods for carrying out cell-cell fusion are described, for example, in Ogle et al., Nat. Rev. Mol. Cell Biol, 6; 567-75 (2005) and Zhou et al., Cell Stem Cell, 3; 382-388 (2008). Examples of methods for carrying out SCNT are described, for example, in Hanna et al., Cell, 143; 508-525 (2010); Stadtfeld et al., Genes Dev., 24; 2239-2263 (2010); Wilmut et al., Nature, 385; 810-813 (1997); Vizcardo et al., Cell Stem Cell, 12; 31-36 (2013); and Crompton et al., Cell Stem Cell, 12; 6-8 (2013). In an aspect of the invention, the host cell is an iPSC that was prepared by reprogramming, any of the host cells described herein (e.g., T cells, NK cells, or invariant natural killer T cells) to a pluripotent state.

Also provided by the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, aa neutrophil, an erythrocyte, a hepatocyte, an endothelial cell, an epithelial cells, a muscle cell. a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one aspect of the invention, the population of cells is a clonal population comprising host cells comprising a recombinant expression vector as described herein.

In an aspect of the invention, the numbers of cells in the population may be rapidly expanded. Expansion of the numbers of T cells can be accomplished by any of a number of methods as are known in the art as described in, for example, U.S. Pat. No, 8,034,334; U.S. Pat. Nos, 8,383,099; 11,401,503; Dudley et al., J. Immunother . . . 26; 332-42 (2003); and Riddell et al., J. Immunol. Methods, 128; 189-201 (1990). In an aspect, expansion of the numbers of T cells is carried out by culturing the T cells with OKT3 antibody. IL-2, and feeder PBMC (e.g., irradiated allogeneic PBMC).

The inventive TCRs, polypeptides, proteins, nucleic acids, recombinant expression vectors, and host cells (including populations thereof), can be isolated and/or purified. The term “isolated,” as used herein, means having been removed from its natural environment. The term “purified,” as used herein, means having been increased in purity, wherein “purity” is a relative term, and not to be necessarily construed as absolute purity. For example, the purity can be at least about 50%, can be greater than about 60%, about 70%, about 80%, about 90%, about 95%, or can be about 100%.

The inventive TCRs, polypeptides, proteins, bispecific engager TCR fusion proteins, nucleic acids, recombinant expression vectors, and host cells (including populations thereof), all of which are collectively referred to as “inventive TCR materials” hereinafter, can be formulated into a composition, such as a pharmaceutical composition. In this regard, the invention provides a pharmaceutical composition comprising any of the TCRs, polypeptides, proteins, bispecific engager TCR fusion proteins, nucleic acids, expression vectors, and host cells (including populations thereof), described herein, and a pharmaceutically acceptable carrier. The inventive pharmaceutical compositions containing any of the inventive TCR materials can comprise more than one inventive TCR material, e.g., a polypeptide and a nucleic acid, or two or more different TCRs. Alternatively, the pharmaceutical composition can comprise an inventive TCR material in combination with another pharmaceutically active agent(s) or drug(s), such as a chemotherapeutic agents, e.g., asparaginase. busulfan, carboplatin, cisplatin, daunorubicin, doxorubicin, fluorouracil, gemcitabine, hydroxyurea, methotrexate, paclitaxel, rituximab, vinblastine, vincristine, etc.

Preferably, the carrier is a pharmaceutically acceptable carrier. With respect to pharmaceutical compositions, the carrier can be any of those conventionally used for the particular inventive TCR material under consideration. Methods for preparing administrable compositions are known or apparent to those skilled in the art and are described in more detail in, for example, Remington; The Science and Practice of Pharmacy, 23^{rd} Ed. . . . Pharmaceutical Press (2020). It is preferred that the pharmaceutically acceptable carrier be one which has no detrimental side effects or toxicity under the conditions of use.

The choice of carrier will be determined in part by the particular inventive TCR material, as well as by the particular method used to administer the inventive TCR material. Accordingly, there are a variety of suitable formulations of the pharmaceutical composition of the invention. Suitable formulations may include any of those for parenteral, subcutaneous, intravenous, intramuscular, intraarterial, intrathecal, intratumoral, or interperitoneal administration. More than one route can be used to administer the inventive TCR materials, and in certain instances, a particular route can provide a more immediate and more effective response than another route.

Preferably, the inventive TCR material is administered by injection, e.g., intravenously. When the inventive TCR material is a host cell (or population thereof) expressing the inventive TCR, the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter. Deerfield, IL), about 5% dextrose in water, or Ringer's lactate. In an aspect, the pharmaceutically acceptable carrier is supplemented with human serum albumen.

For purposes of the invention, the amount or dose (e.g., numbers of cells when the inventive TCR material is one or more cells) of the inventive TCR material administered should be sufficient to effect, e.g., a therapeutic or prophylactic response, in the subject or animal over a reasonable time frame. For example, the dose of the inventive TCR material should be sufficient to bind to a cancer antigen (e.g., mutated CDKN2A peptide), or detect, treat or prevent cancer in a period of from about 2 hours or longer, e.g., 12 to 24 or more hours, from the time of administration. In certain aspects, the time period could be even longer. The dose will be determined by the efficacy of the particular inventive TCR material and the condition of the animal (e.g., human), as well as the body weight of the animal (e.g., human) to be treated.

Many assays for determining an administered dose are known in the art. For purposes of the invention, an assay, which comprises comparing the extent to which target cells are lysed or IFN-γ is secreted by T cells expressing the inventive TCR, polypeptide, or protein upon administration of a given dose of such T cells to a mammal among a set of mammals of which each is given a different dose of the T cells, could be used to determine a starting dose to be administered to a mammal. The extent to which target cells are lysed or IFN-γ is secreted upon administration of a certain dose can be assayed by methods known in the art.

The dose of the inventive TCR material also will be determined by the existence, nature and extent of any adverse side effects that might accompany the administration of a particular inventive TCR material. Typically, the attending physician will decide the dosage of the inventive TCR material with which to treat each individual patient, taking into consideration a variety of factors, such as age, body weight, general health, diet, sex, inventive TCR material to be administered, route of administration, and the severity of the cancer being treated. In an aspect in which the inventive TCR material is a population of cells, the number of cells administered per infusion may vary, e.g., from about 1×10⁶ to about 1×10¹² cells or more. In certain aspects, fewer than 1×10⁶ cells may be administered.

One of ordinary skill in the art will readily appreciate that the inventive TCR materials of the invention can be modified in any number of ways, such that the therapeutic or prophylactic efficacy of the inventive TCR materials is increased through the modification. For instance, the inventive TCR materials can be conjugated either directly or indirectly through a bridge to a chemotherapeutic agent. The practice of conjugating compounds to a chemotherapeutic agent is known in the art. One of ordinary skill in the art recognizes that sites on the inventive TCR materials, which are not necessary for the function of the inventive TCR materials, are suitable sites for attaching a bridge and/or a chemotherapeutic agent, provided that the bridge and/or chemotherapeutic agent, once attached to the inventive TCR materials, do(es) not interfere with the function of the inventive TCR materials, i.e., the ability to bind to mutated CDKN2A peptide or to detect, treat, or prevent cancer.

It is contemplated that the inventive pharmaceutical compositions, TCRs, polypeptides, proteins, bispecific engager TCR fusion proteins, nucleic acids, recombinant expression vectors, host cells, and populations of cells can be used in methods of treating or preventing cancer. Without being bound to a particular theory, the inventive TCRs are believed to bind specifically to mutated CDKN2A peptide, such that the TCR (or related inventive polypeptide or protein), when expressed by a cell, is able to mediate an immune response against a target cell expressing mutated CDKN2A peptide. In this regard, an aspect of the invention provides a method of treating or preventing cancer in a mammal, comprising administering to the mammal any of the pharmaceutical compositions, TCRs, polypeptides, proteins, or bispecific engager TCR fusion proteins described herein, any nucleic acid or recombinant expression vector comprising a nucleotide sequence encoding any of the TCRs, polypeptides, proteins, or bispecific engager TCR fusion proteins described herein, or any host cell or population of cells comprising a recombinant vector which encodes any of the TCRs, polypeptides, proteins, or bispecific engager TCR fusion proteins described herein, in an amount effective to treat or prevent cancer in the mammal.

An aspect of the invention provides a method of inducing an immune response against a cancer in a mammal, comprising administering to the mammal any of the pharmaceutical compositions, TCRs, polypeptides, proteins, or bispecific engager TCR fusion proteins described herein, any nucleic acid or recombinant expression vector comprising a nucleotide sequence encoding any of the TCRs, polypeptides, proteins, or bispecific engager TCR fusion proteins described herein, or any host cell or population of cells comprising a recombinant vector which encodes any of the TCRs, polypeptides. proteins, or bispecific engager TCR fusion proteins described herein, in an amount effective to induce an immune response against the cancer in the mammal.

An aspect of the invention provides any of the pharmaceutical compositions, TCRs, polypeptides, proteins, or bispecific engager TCR fusion proteins, described herein, any nucleic acid or recombinant expression vector comprising a nucleotide sequence encoding any of the TCRs, polypeptides, proteins, or bispecific engager TCR fusion proteins described herein, or any host cell or population of cells comprising a recombinant vector which encodes any of the TCRs, polypeptides, proteins, or bispecific engager TCR fusion proteins described herein, for use in the treatment or prevention of cancer in a mammal.

An aspect of the invention provides any of the pharmaceutical compositions, TCRs, polypeptides, proteins, or bispecific engager TCR fusion proteins described herein, any nucleic acid or recombinant expression vector comprising a nucleotide sequence encoding any of the TCRs, polypeptides, proteins, or bispecific engager TCR fusion proteins described herein, or any host cell or population of cells comprising a recombinant vector which encodes any of the TCRs, polypeptides, proteins, or bispecific engager TCR fusion proteins described herein, for use in inducing an immune response against a cancer in a mammal.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the cancer being treated or prevented. For example, treatment or prevention can include promoting the regression of a tumor. Also, for purposes herein, “prevention” can encompass delaying the onset of the cancer, or a symptom or condition thereof. Alternatively or additionally, “prevention” may encompass preventing or delaying the recurrence of cancer, or a symptom or condition thereof.

Also provided is a method of detecting the presence of cancer in a mammal. The method comprises (i) contacting a sample comprising one or more cells from the mammal with any of the inventive TCRs, polypeptides, proteins, bispecific engager TCR fusion proteins, nucleic acids, recombinant expression vectors, host cells, populations of cells, or pharmaceutical compositions described herein, thereby forming a complex, and (ii) detecting the complex, wherein detection of the complex is indicative of the presence of cancer in the mammal.

With respect to the inventive method of detecting cancer in a mammal, the sample of cells can be a sample comprising whole cells, lysates thereof, or a fraction of the whole cell lysates, e.g., a nuclear or cytoplasmic fraction, a whole protein fraction, or a nucleic acid fraction.

For purposes of the inventive method of detecting cancer, the contacting can take place in vitro or in vivo with respect to the mammal. Preferably, the contacting is in vitro.

Also, detection of the complex can occur through any number of ways known in the art. For instance, the inventive TCRs, polypeptides, proteins, nucleic acids, recombinant expression vectors, host cells, or populations of cells, described herein, can be labeled with a detectable label such as, for instance, a radioisotope, a fluorophore (e.g., fluorescein isothiocyanate (FITC), phycoerythrin (PE)), an enzyme (e.g., alkaline phosphatase. horseradish peroxidase), and element particles (e.g., gold particles).

For purposes of the inventive methods, wherein host cells or populations of cells are administered, the cells can be cells that are allogeneic or autologous to the mammal. Preferably, the cells are autologous to the mammal.

With respect to the inventive methods, the cancer can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, colorectal cancer, endometrial cancer, esophageal cancer, uterine cervical cancer, gastrointestinal carcinoid tumor, glioma. Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx. ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer. A preferred cancer is melanoma, pancreatic cancer, or gastrointestinal cancer. In an aspect of the invention, the cancer expresses a mutated amino acid sequence encoded by mutated CDKN2A. The mutated CDKN2A expressed by the cancer may be as described herein with respect to other aspects of the invention.

The mammal referred to in the inventive methods can be any mammal. As used herein, the term “mammal” refers to any mammal, including, but not limited to, mammals of the order Rodentia, such as mice and hamsters, and mammals of the order Lagomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

The following examples further illustrate the invention but, of course, should not be construed as in any waylimiting its scope.

### EXAMPLE 1

This example demonstrates the isolation of TCRs from Patient 3333.

Tumor infiltrating lymphocytes (TIL) were isolated from Patient 3333 with metastatic melanoma. Patient 3333 expressed the HLA-A*03; 01 allele and a frameshift mutation in the CDKN2A gene (c.304delG, p.A102fs). The mutated amino acid sequences encoded by the mutated CDKN2A gene in Patient 3333 are shown in Table 4. In Table 4, the open reading frame (orf) 3 encoding the T cell epitope AVCPWTWLR (SEQ ID NO: 141) is underlined. The mutated amino acid sequence of SEQ ID NO: 150 includes portions of WT p16INK4a and WT p14ARF; Amino acid residues 116-170 of the mutated amino acid sequence of SEQ ID NO: 150 are 100% identical to amino acid residues 102-156 of WT p16INK4a SEQ ID NO: 147. Amino acid residues 1-115 of the mutated amino acid sequence of SEQ ID NO: 150 are 100% identical to amino acid residues 1-115 of WT p14ARF SEQ ID NO: 148.

TABLE 4

| | Mutated Amino Acid Sequence |
|---|---|
| Protein | Encoded by Mutated CDKN2A gene |
| p14ARF/p16INK4a | MVRRFLVTLRIRRACGPPRVRVFVVHIPRLTGEWAAPGAPA |
| | AVALVLMLLRSQRLGQQPLPRRPGHDDGQRPSGGAAAAPR |
| | RGAQLRRPRHSHPTRARRCPGGLPGHAGGAAPGRARLDVR |
| | DAWGRLPVDLAEELGHRDVARYLRAAAGGTRGSNHARID |
| | AAEGPSDIPD (SEQ ID NO: 150) |
| p16INK4a | MEPAAGSSMEPSADWLATAAARGRVEEVRALLEAGALPN |
| orf3 | APNSYGRRPIQVMMMGSARVAELLLLHGAEPNCADPATLT |
| | RPVHDAAREGFLDTLVVLHRAGRGWTCAMPGAVCPWTW |
| | LRSWAIAMSHGTCARLRGAPEAVTMPA (SEQ ID NO: 151) |

Tetramer staining technology with the fluorophores PE (phycoerythrin) and APC (allophycocyanin) was utilized to test whether the TIL were reactive against the mutated peptide AVCPWTWLR (SEQ ID NO: 141). The tetramer was conjugated to both of these fluorophores or to neither fluorophore (negative control). The isolated TIL, which had been previously determined to recognize autologous tumor, were stained with the tetramer AVCPWTWLR (SEQ ID NO: 141).

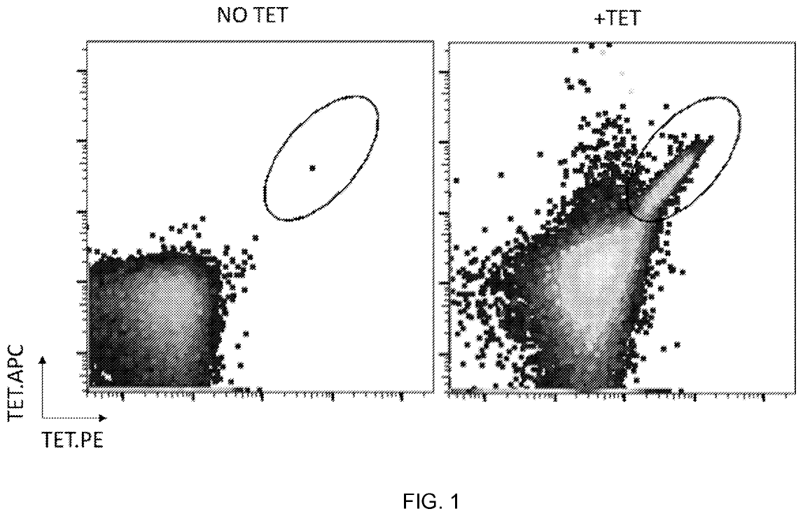

The results for the tetramer conjugated to both fluorophores are shown in FIG. 1 (right plot). The results for the negative control are shown in FIG. 1 (left plot). As shown in FIG. 1, anti-tumor TIL stained with the tetramer.

The tetramer-sorted cells were sequenced using a single cell sequencing method. Four TCRs were found to be expressed by the tetramer-sorted cells; 3333 TCR-A (alpha (TRAV 12-2)/beta (TRBV 24-1)), 3333 TCR-B, 3333 TCR-C(alpha (TRAV 12-2)/beta (TRBV 25-1)), and 3333 TCR-D. To facilitate cloning of the TCR expression cassette into the MSGVI vector 5′Ncol site, the second amino acid in the N-terminal signal peptide of the TCRVβ chain was changed to an alanine (A). The alpha and beta chain variable region amino acid sequences for 3333 TCR-A and 3333 TCR-C are shown in Table 5. The CDRs are underlined. The N-terminal signal peptides are in bold font.

TABLE 5

| | | Amino acid sequence |
|---|---|---|
| TCR Name | TCR chain | (complementarity determining regions are underlined) |
| 3333 TCR-A | Variable α | QQKEVEQNSGPLSVPEGAIASLNCTYSDRGSQSFFWYRQY |
| | (Predicted | SGKSPELIMSIYSNGDKEDGRFTAQLNKASQYVSLLIRDSQP |
| | sequence | SDSATYLCAVNTASGTYKYIFGTGTRLKVLA (SEQ ID NO: 7) |
| | without N- | |
| | terminal signal | |
| | peptide) | |
| | Variable β | VTQTPRNRITKTGKRIMLECSQTKGHDRMYWYRQDPGLGL |
| | (Predicted | RLIYYSFDVKDINKGEISDGYSVSRQAQAKFSLSLESAIPNQT |
| | sequence | ALYFCATSDFRGKPQHFGDGTRLSIL (SEQ ID NO: 8) |
| | without N- | |
| | terminal signal | |
| | peptide) | |
| | Variable α | MMKSLRVLLVILWLQLSWVWSQQKEVEQNSGPLSVPEGAI |
| | (With N- | ASLNCTYSDRGSQSFFWYRQYSGKSPELIMSIYSNGDKEDG |
| | terminal signal | RFTAQLNKASQYVSLLIRDSQPSDSATYLCAVNTASGTYKYI |
| | peptide) | FGTGTRLKVLA (SEQ ID NO: 9) |
| | Variable β | MASLLFFCGAFYLLGTGSMDADVTQTPRNRITKTGKRIMLE |
| | (With N- | CSQTKGHDRMYWYRQDPGLGLRLIYYSFDVKDINKGEISDG |
| | terminal signal | YSVSRQAQAKFSLSLESAIPNQTALYFCATSDFRGKPQHFG |
| | peptide) | DGTRLSIL (SEQ ID NO: 10) |
| 3333 TCR-C | Variable α | QQKEVEQNSGPLSVPEGAIASLNCTYSDRGSQSFFWYRQY |
| | (Predicted | SGKSPELIMSIYSNGDKEDGRFTAQLNKASQYVSLLIRDSQP |
| | sequence | SDSATYLCAVNRAREAAGNKLTFGGGTRVLVKP (SEQ ID |
| | without N- | NO: 17) |
| | terminal signal | |
| | peptide) | |
| | Variable β | DIYQTPRYLVIGTGKKITLECSQTMGHDKMYWYQQDPGMEL |
| | (Predicted | HLIHYSYGVNSTEKGDLSSESTVSRIRTEHFPLTLESARPSH |
| | sequence | TSQYLCASSRPGSEKLFFGSGTQLSVL (SEQ ID NO: 18) |
| | without N- | |
| | terminal signal | |
| | peptide) | |
| | Variable α | MMKSLRVLLVILWLQLSWVWSQQKEVEQNSGPLSVPEGAI |
| | (With N- | ASLNCTYSDRGSQSFFWYRQYSGKSPELIMSIYSNGDKEDG |
| | terminal signal | RFTAQLNKASQYVSLLIRDSQPSDSATYLCAVNRAREAAGN |
| | peptide) | KLTFGGGTRVLVKP (SEQ ID NO: 19) |
| | Variable β | MAIRLLCYMGFYFLGAGLMEADIYQTPRYLVIGTGKKITLEC |
| | (With N- | SQTMGHDKMYWYQQDPGMELHLIHYSYGVNSTEKGDLSSE |
| | terminal signal | STVSRIRTEHFPLTLESARPSHTSQYLCASSRPGSEKLFFGS |
| | peptide)) | GTQLSVL (SEQ ID NO: 20) |

### EXAMPLE 2

This example demonstrates the isolation of a TCR from an infusion product used to treat Patient 1913.

Autologous TIL were isolated from an infusion product used to treat Patient 1913 with metastatic melanoma. Patient 1913 expressed the HLA-A+011; 01 allele and a frameshift mutation in the CDKN2A gene (c.222_223delCG, c.G220T). The mutated amino acid sequences encoded by the mutated CDKN2A gene in Patient 1913 are shown in Table 6. In Table 6, the open reading frame (orf) 3 encoding the T cell epitope AVCPWTWLR (SEQ ID NO: 141) is underlined. The mutated amino acid sequence of SEQ ID NO: 153 includes portions of WT p16INK4a and WT p14ARF; Amino acid residues 60-117 of the mutated amino acid sequence of SEQ ID NO: 153 are 100% identical to amino acid residues 75-132 of WT p14ARF SEQ ID NO: 148. Amino acid residues 1-59 of the mutated amino acid sequence of SEQ ID NO: 153 are 100% identical to amino acid residues 1-59 of WT p16INK4a SEQ ID NO: 147.

TABLE 6

| | Mutated Amino Acid Sequence |
|---|---|
| Protein | Encoded by Mutated CDKN2A gene |
| p14ARF | MVRRFLVTLRIRRACGPPRVRVFVVHIPRLTGEWAAPGAPA |
| orf3 | AVALVLMLLRSQRLGQQPLPRRPGHDDGQRPSWSCCCSTA |
| | RSPTAPTPPLSPDPCTTLPGRASWTRWWCCTGPGRGWTCA |
| | MPGAVCPWTWLRSWAIAMSHGTCARLRGAPEAVTMPA |
| | (SEQ ID NO: 152) |
| p16INK4a/p14arf | MEPAAGSSMEPSADWLATAAARGRVEEVRALLEAGALPN |
| | APNSYGRRPIQVMMMGSARVGAAAAPRRGAQLRRPRHSH |
| | PTRARRCPGGLPGHAGGAAPGRGAAGRARCLGPSARGPG |
| | (SEQ ID NO: 153) |

Tetramer staining technology with the fluorophores PE and APC was utilized to test whether the TIL were reactive against the mutated peptide AVCPWTWLR (SEQ ID NO: 141). The tetramer was conjugated to both of these fluorophores or to neither fluorophore (negative control). The isolated TIL, which had been previously determined to recognize autologous tumor, were stained with the tetramer AVCPWTWLR (SEQ ID NO: 141). The anti-tumor TIL stained with the tetramer.

The tetramer-sorted cells were sequenced using a single cell sequencing method. One TCR was found to be expressed by the tetramer-sorted cells; 1913 TCR-41BB (alpha (TRAV 39)/beta (TRBV 9)). To facilitate cloning of the TCR expression cassette into the MSGVI vector 5′Ncol site, the second amino acid in the N-terminal signal peptide of the TCRVβ chain was changed to an alanine (A). The alpha and beta chain variable region amino acid sequences for 1913 TCR-41BB are shown in Table 7. The CDRs are underlined. The N-terminal signal peptides are in bold font.

TABLE 7

| | | Amino acid sequence |
|---|---|---|
| TCR Name | TCR chain | (complementarity determining regions are underlined) |
| 1913 TCR- | Variable α | ELKVEQNPLFLSMQEGKNYTIYCNYSTTSDRLYWYRQDPGK |
| 41BB | (Predicted | SLESLFVLLSNGAVKQEGRLMASLDTKARLSTLHITAAVHDL |
| | sequence | SATYFCAVDPTGANSKLTFGKGITLSVRP (SEQ ID NO: 27) |
| | without N- | |
| | terminal signal | |
| | peptide) | |
| | Variable β | GVTQTPKHLITATGQRVTLRCSPRSGDLSVYWYQQSLDQGL |
| | (Predicted | QFLIQYYNGEERAKGNILERFSAQQFPDLHSELNLSSLELGD |
| | sequence | SALYFCASSVAISGEETQYFGPGTRLLVL (SEQ ID NO: 28) |
| | without N- | |
| | terminal signal | |
| | peptide) | |
| | Variable α | MKKLLAMILWLQLDRLSGELKVEQNPLFLSMQEGKNYTIYC |
| | (With N- | NYSTTSDRLYWYRQDPGKSLESLFVLLSNGAVKQEGRLMA |
| | terminal signal | SLDTKARLSTLHITAAVHDLSATYFCAVDPTGANSKLTFGKGI |
| | peptide) | TLSVRP (SEQ ID NO: 29) |
| | Variable β | MAFRLLCCVAFCLLGAGPVDSGVTQTPKHLITATGQRVTLR |
| | (With N- | CSPRSGDLSVYWYQQSLDQGLQFLIQYYNGEERAKGNILER |
| | terminal signal | FSAQQFPDLHSELNLSSLELGDSALYFCASSVAISGEETQYF |
| | peptide) | GPGTRLLVL (SEQ ID NO: 30) |

### EXAMPLE 3

This example demonstrates the isolation of TCRs from the peripheral blood of Patient 4286.

Patient 4286, with metastatic melanoma, had a tumor that expressed the P114L p16INK4a mutation. A peripheral blood sample was obtained from Patient 4286. A CD8+CD45RO+CD45RA-HLADR+CD39+CD103+anti-tumor T cell fraction was separated from the peripheral blood sample using fluorescence-activated cell sorting (FACS). The separated anti-tumor T cell fraction was cultured for about 12 days.

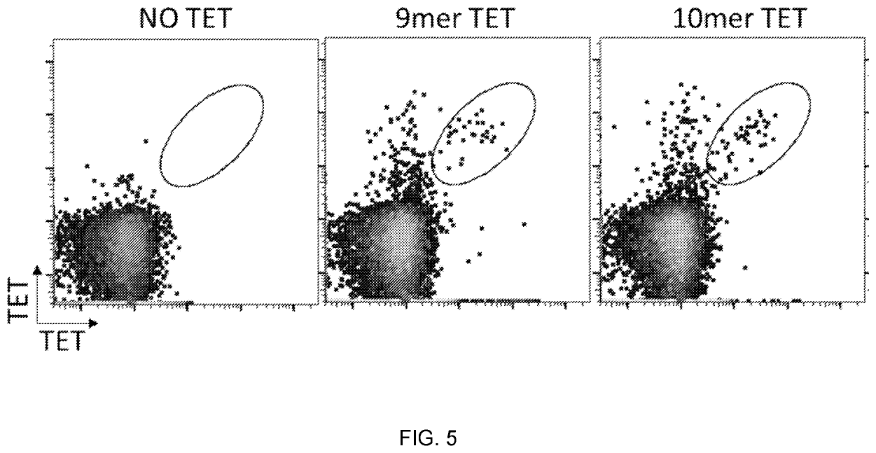

Tetramer staining technology was utilized to test whether the T cells in the separated anti-tumor T cell fraction were reactive against the mutated P114L p16INK4a 9-mer peptide LLVDLAEEL (SEQ ID NO: 142) or the mutated CDKN2A 10-mer peptide RLLVDLAEEL (SEQ ID NO: 143). The T cells in the separated anti-tumor T cell fraction were stained with the 9-mer LLVDLAEEL (SEQ ID NO: 142) tetramer, 10-mer RLLVDLAEEL (SEQ ID NO: 143) tetramer, or no tetramer. The tetramer was conjugated to fluorophore PE and APC. Staining the cells with both tetramers increases the confidence of the specificity. The results are shown in FIG. 5. The results show dual tetramer stained CD8+ T cells isolated from peripheral blood of cancer patient.

The tetramer-sorted cells were sequenced using a single cell sequencing method. Four TCRs were found to be expressed by the tetramer-sorted cells; 4286 TCR 10-1, 4286 TCR 10-2, 4286 TCR 10-3, and 4286 TCR 10-4. To facilitate cloning of the TCR expression cassette into the MSGV1 vector 5′Ncol site, the second amino acid in the N-terminal signal peptide of the 4286 TCR 10-1 TCRVβ chain and the 4286 TCR 10-2 TCRVβ chain were each changed to an alanine (A). The alpha and beta chain variable region amino acid sequences for these TCRs are shown in Table 8. The CDRs are underlined. The N-terminal signal peptides are in bold font.

TABLE 8

| | | Amino acid sequence |
|---|---|---|
| TCR Name | TCR chain | (complementarity determining regions are underlined) |
| 4286 TCR 10-1 | Variable α | QQKEVEQNSGPLSVPEGAIASLNCTYSDRGSQSFFWYRQY |
| | (Predicted | SGKSPELIMFIYSNGDKEDGRFTAQLNKASQYVSLLIRDSQP |
| | sequence | SDSATYLCAVKSWVIFGKGTRLHILP (SEQ ID NO: 37) |
| | without N- | |
| | terminal signal | |
| | peptide) | |
| | Variable β | GVTQSPTHLIKTRGQQVTLRCSPISGHKSVSWYQQVLGQGP |
| | (Predicted | QFIFQYYEKEERGRGNFPDRFSARQFPNYSSELNVNALLLG |
| | sequence | DSALYLCASSPSAQSSYGYTFGSGTRLTVV (SEQ ID NO: |
| | without N- | 38) |
| | terminal signal | |
| | peptide) | |
| | Variable α | MKSLRVLLVILWLQLSWVWSQQKEVEQNSGPLSVPEGAIA |
| | (With N- | SLNCTYSDRGSQSFFWYRQYSGKSPELIMFIYSNGDKEDGR |
| | terminal signal | FTAQLNKASQYVSLLIRDSQPSDSATYLCAVKSWVIFGKGTR |
| | peptide) | LHILP (SEQ ID NO: 39) |
| | Variable β | MAPGLLCWVLLCLLGAGPVDAGVTQSPTHLIKTRGQQVTL |
| | (With N- | RCSPISGHKSVSWYQQVLGQGPQFIFQYYEKEERGRGNFP |
| | terminal signal | DRFSARQFPNYSSELNVNALLLGDSALYLCASSPSAQSSYG |
| | peptide) | YTFGSGTRLTVV (SEQ ID NO: 40) |
| 4286 TCR 10-2 | Variable α | QQKEVEQNSGPLSVPEGAIASLNCTYSDRGSQSFFWYRQY |
| | (Predicted | SGKSPELIMFIYSNGDKEDGRFTAQLNKASQYVSLLIRDSQP |
| | sequence | SDSATYLCAVPNSGYSTLTFGKGTMLLVSP (SEQ ID NO: 47) |
| | without N- | |
| | terminal signal | |
| | peptide) | |
| | Variable β | GVTQSPTHLIKTRGQQVTLRCSPISGHKSVSWYQQVLGQGP |
| | (Predicted | QFIFQYYEKEERGRGNFPDRFSARQFPNYSSELNVNALLLG |
| | sequence | DSALYLCASRTGTVTNSYNEQFFGPGTRLTVL (SEQ ID NO: |
| | without N- | 48) |
| | terminal signal | |
| | peptide) | |
| | Variable α | MKSLRVLLVILWLQLSWVWSQQKEVEQNSGPLSVPEGAIA |
| | (With N- | SLNCTYSDRGSQSFFWYRQYSGKSPELIMFIYSNGDKEDGR |
| | terminal signal | FTAQLNKASQYVSLLIRDSQPSDSATYLCAVPNSGYSTLTFG |
| | peptide) | KGTMLLVSP (SEQ ID NO: 49) |
| | Variable β | MAPGLLCWVLLCLLGAGPVDAGVTQSPTHLIKTRGQQVTL |
| | (With N- | RCSPISGHKSVSWYQQVLGQGPQFIFQYYEKEERGRGNFP |
| | terminal signal | DRFSARQFPNYSSELNVNALLLGDSALYLCASRTGTVTNSY |
| | peptide)) | NEQFFGPGTRLTVL (SEQ ID NO: 50) |
| 4286 TCR 10-3 | Variable α | QSVSQHNHHVILSEAASLELGCNYSYGGTVNLFWYVQYPG |
| | (Predicted | QHLQLLLKYFSGDPLVKGIKGFEAEFIKSKFSFNLRKPSVQW |
| | sequence | SDTAEYFCAVKDTGFQKLVFGTGTRLLVSP (SEQ ID NO: 57) |
| | without N- | |
| | terminal signal | |
| | peptide) | |
| | Variable β | GSGLGAVVSQHPSWVICKSGTSVKIECRSLDFQATTMFWYR |
| | (Predicted | QFPKQSLMLMATSNEGSKATYEQGVEKDKFLINHASLTLSTL |
| | sequence | TVTSAHPEDSSFYICSARGGRNSFEQFFGPGTRLTVL (SEQ |
| | without N- | ID NO: 58) |
| | terminal signal | |
| | peptide) | |
| | Variable α | MLLLLIPVLGMIFALRDARAQSVSQHNHHVILSEAASLELGC |
| | (With N- | NYSYGGTVNLFWYVQYPGQHLQLLLKYFSGDPLVKGIKGFE |
| | terminal signal | AEFIKSKFSFNLRKPSVQWSDTAEYFCAVKDTGFQKLVFGT |
| | peptide) | GTRLLVSP (SEQ ID NO: 59) |
| | Variable β | MLLLLLLLGPGISLLLPGSLAGSGLGAVVSQHPSWVICKSG |
| | (With N- | TSVKIECRSLDFQATTMFWYRQFPKQSLMLMATSNEGSKAT |
| | terminal signal | YEQGVEKDKFLINHASLTLSTLTVTSAHPEDSSFYICSARGG |
| | peptide)) | RNSFEQFFGPGTRLTVL (SEQ ID NO: 60) |
| 4286 TCR 10-4 | Variable α | QSVSQHNHHVILSEAASLELGCNYSYGGTVNLFWYVQYPG |
| | (Predicted | QHLQLLLKYFSGDPLVKGIKGFEAEFIKSKFSFNLRKPSVQW |
| | sequence | SDTAEYFCAVKDTGFQKLVFGTGTRLLVSP (SEQ ID NO: 67) |
| | without N- | |
| | terminal signal | |
| | peptide) | |
| | Variable β | GSGLGAVVSQHPSWVICKSGTSVKIECRSLDFQATTMFWYR |
| | (Predicted | QFPKQSLMLMATSNEGSKATYEQGVEKDKFLINHASLTLSTL |
| | sequence | TVTSAHPEDSSFYICSARGGARSYEQYFGPGTRLTVT (SEQ |
| | without N- | ID NO: 68) |
| | terminal signal | |
| | peptide) | |
| | Variable α | MLLLLIPVLGMIFALRDARAQSVSQHNHHVILSEAASLELGC |
| | (With N- | NYSYGGTVNLFWYVQYPGQHLQLLLKYFSGDPLVKGIKGFE |
| | terminal signal | AEFIKSKFSFNLRKPSVQWSDTAEYFCAVKDTGFQKLVFGT |
| | peptide) | GTRLLVSP (SEQ ID NO: 69) |
| | Variable β | MLLLLLLLGPGISLLLPGSLAGSGLGAVVSQHPSWVICKSG |
| | (With N- | TSVKIECRSLDFQATTMFWYRQFPKQSLMLMATSNEGSKAT |
| | terminal signal | YEQGVEKDKFLINHASLTLSTLTVTSAHPEDSSFYICSARGG |
| | peptide)) | ARSYEQYFGPGTRLTVT (SEQ ID NO: 70) |

### EXAMPLE 4

This example demonstrates the construction of retroviral vectors encoding the respective TCRs of Tables 5, 7, and 8.

Nucleotide sequences encoding the variable regions of the α and β chains of the TCRs of Tables 5, 7, and 8 were obtained and codon-optimized. The TCRB VDJ regions were fused to the mouse TCR constant chain. The TCRα VJ regions were fused to the mouse TCRα constant chain. Without being bound to a particular theory or mechanism, it is believed that replacing the constant regions of the human TCRα and TCRβ chains with the corresponding murine constant regions improves TCR expression and functionality (Cohen et al., Cancer Res., 66 (17); 8878-86 (2006)).

In addition, the murine TCRα and TCRβ constant chains were cysteine-modified. Transmembrane hydrophobic mutations were introduced into the murine TCRα constant chain. Without being bound to a particular theory or mechanism, it is believed that these modifications result in preferential pairing of the introduced TCR chains and enhanced TCR surface expression and functionality (Cohen et al., Cancer Res., 67 (8); 3898-903 (2007); Haga-Friedman et al., J. Immu., 188; 5538-5546 (2012)). The full length α and β chains of each of the TCRs, including these modifications to the constant region, are shown in Table D. In Table 9, the CDRs are underlined, and the modified amino acid residues of the constant region are underlined and in bold. The N-terminal signal peptides are in bold font.

TABLE 9

| SEQ ID NO: | Sequence |
|---|---|
| SEQ ID NO: 81 | MMKSLRVLLVILWLQLSWVWSQQKEVEQNSGPLSVPEGAIASLNCTYS |
| (Cys-substituted, LVL- | DRGSQSFFWYRQYSGKSPELIMSIYSNGDKEDGRFTAQLNKASQYVSLLI |
| modified 3333 TCR-A α | RDSQPSDSATYLCAVNTASGTYKYIFGTGTRLKVLANIQNPEPAVYQLKD |
| chain with N-terminal | PRSQDSTLCLFTDFDSQINVPKTMESGTFITDKCVLDMKAMDSKSNGAIA |
| signal peptide) | WSNQTSFTCQDIFKETNATYPSSDVPCDATLTEKSFETDMNLNFQNLLVI |
| | VLRILLLKVAGFNLLMTLRLWSS |
| SEQ ID NO: 82 | MASLLFFCGAFYLLGTGSMDADVTQTPRNRITKTGKRIMLECSQTKGH |
| (Cys-substituted, LVL- | DRMYWYRQDPGLGLRLIYYSFDVKDINKGEISDGYSVSRQAQAKFSLSL |
| modified 3333 TCR-A β | ESAIPNQTALYFCATSDFRGKPQHFGDGTRLSILEDLRNVTPPKVSLFEPS |
| chain with N-terminal | KAEIANKQKATLVCLARGFFPDHVELSWWVNGKEVHSGVCTDPQAYKE |
| signal peptide) | SNYSYCLSSRLRVSATFWHNPRNHFRCQVQFHGLSEEDKWPEGSPKPVT |
| | QNISAEAWGRADCGITSASYQQGVLSATILYEILLGKATLYAVLVSTLVV |
| | MAMVKRKNS |
| SEQ ID NO: 83 | QQKEVEQNSGPLSVPEGAIASLNCTYSDRGSQSFFWYRQYSGKSPELIMSI |
| (Cys-substituted, LVL- | YSNGDKEDGRFTAQLNKASQYVSLLIRDSQPSDSATYLCAVNTASGTYK |
| modified 3333 TCR-A α | YIFGTGTRLKVLANIQNPEPAVYQLKDPRSQDSTLCLFTDFDSQINVPKTM |
| chain predicted sequence | ESGTFITDKCVLDMKAMDSKSNGAIAWSNQTSFTCQDIFKETNATYPSSD |
| without N-terminal | VPCDATLTEKSFETDMNLNFQNLLVIVLRILLLKVAGFNLLMTLRLWSS |
| signal peptide) | |
| SEQ ID NO: 84 | VTQTPRNRITKTGKRIMLECSQTKGHDRMYWYRQDPGLGLRLIYYSFDV |
| (Cys-substituted, LVL- | KDINKGEISDGYSVSRQAQAKFSLSLESAIPNQTALYFCATSDFRGKPQHF |
| modified 3333 TCR-A β | GDGTRLSILEDLRNVTPPKVSLFEPSKAEIANKQKATLVCLARGFFPDHVE |
| chain predicted sequence | LSWWVNGKEVHSGVCTDPQAYKESNYSYCLSSRLRVSATFWHNPRNHF |
| without N-terminal | RCQVQFHGLSEEDKWPEGSPKPVTQNISAEAWGRADCGITSASYQQGVL |
| signal peptide) | SATILYEILLGKATLYAVLVSTLVVMAMVKRKNS |
| SEQ ID NO: 89 | MMKSLRVLLVILWLQLSWVWSQQKEVEQNSGPLSVPEGAIASLNCTYS |
| (Cys-substituted, LVL- | DRGSQSFFWYRQYSGKSPELIMSIYSNGDKEDGRFTAQLNKASQYVSLLI |
| modified 3333 TCR-C α | RDSQPSDSATYLCAVNRAREAAGNKLTFGGGTRVLVKPNIQNPEPAVYQ |
| chain with N-terminal | LKDPRSQDSTLCLFTDFDSQINVPKTMESGTFITDKCVLDMKAMDSKSNG |
| signal peptide) | AIAWSNQTSFTCQDIFKETNATYPSSDVPCDATLTEKSFETDMNLNFQNL |
| | LVIVLRILLLKVAGFNLLMTLRLWSS |
| SEQ ID NO: 90 | MAIRLLCYMGFYFLGAGLMEADIYQTPRYLVIGTGKKITLECSQTMGH |
| (Cys-substituted, LVL- | DKMYWYQQDPGMELHLIHYSYGVNSTEKGDLSSESTVSRIRTEHFPLTLE |
| modified 3333 TCR-C β | SARPSHTSQYLCASSRPGSEKLFFGSGTQLSVLEDLRNVTPPKVSLFEPSK |
| chain with N-terminal | AEIANKQKATLVCLARGFFPDHVELSWWVNGKEVHSGVCTDPQAYKES |
| signal peptide) | NYSYCLSSRLRVSATFWHNPRNHFRCQVQFHGLSEEDKWPEGSPKPVTQ |
| | NISAEAWGRADCGITSASYQQGVLSATILYEILLGKATLYAVLVSTLVVM |
| | AMVKRKNS |
| SEQ ID NO: 91 | QQKEVEQNSGPLSVPEGAIASLNCTYSDRGSQSFFWYRQYSGKSPELIMSI |
| (Cys-substituted, LVL- | YSNGDKEDGRFTAQLNKASQYVSLLIRDSQPSDSATYLCAVNRAREAAG |
| modified 3333 TCR-C α | NKLTFGGGTRVLVKPNIQNPEPAVYQLKDPRSQDSTLCLFTDFDSQINVP |
| chain predicted sequence | KTMESGTFITDKCVLDMKAMDSKSNGAIAWSNQTSFTCQDIFKETNATY |
| without N-terminal | PSSDVPCDATLTEKSFETDMNLNFQNLLVIVLRILLLKVAGFNLLMTLRL |
| signal peptide) | WSS |
| SEQ ID NO: 92 | DIYQTPRYLVIGTGKKITLECSQTMGHDKMYWYQQDPGMELHLIHYSYG |
| (Cys-substituted, LVL- | VNSTEKGDLSSESTVSRIRTEHFPLTLESARPSHTSQYLCASSRPGSEKLFF |
| modified 3333 TCR-C β | GSGTQLSVLEDLRNVTPPKVSLFEPSKAEIANKQKATLVCLARGFFPDHV |
| chain predicted sequence | ELSWWVNGKEVHSGVCTDPQAYKESNYSYCLSSRLRVSATFWHNPRNH |
| without N-terminal | FRCQVQFHGLSEEDKWPEGSPKPVTQNISAEAWGRADCGITSASYQQGV |
| signal peptide) | LSATILYEILLGKATLYAVLVSTLVVMAMVKRKNS |
| SEQ ID NO: 97 | MKKLLAMILWLQLDRLSGELKVEQNPLFLSMQEGKNYTIYCNYSTTSD |
| (Cys-substituted, LVL- | RLYWYRQDPGKSLESLFVLLSNGAVKQEGRLMASLDTKARLSTLHITAA |
| modified 1913 TCR- | VHDLSATYFCAVDPTGANSKLTFGKGITLSVRPNIQNPEPAVYQLKDPRS |
| 41BB α chain with N- | QDSTLCLFTDFDSQINVPKTMESGTFITDKCVLDMKAMDSKSNGAIAWS |
| terminal signal peptide) | NQTSFTCQDIFKETNATYPSSDVPCDATLTEKSFETDMNLNFQNLLVIVL |
| | RILLLKVAGFNLLMTLRLWSS |
| SEQ ID NO: 98 | MAFRLLCCVAFCLLGAGPVDSGVTQTPKHLITATGQRVTLRCSPRSGD |
| (Cys-substituted, LVL- | LSVYWYQQSLDQGLQFLIQYYNGEERAKGNILERFSAQQFPDLHSELNLS |
| modified 1913 TCR- | SLELGDSALYFCASSVAISGEETQYFGPGTRLLVLEDLRNVTPPKVSLFEP |
| 41BB β chain with N- | SKAEIANKQKATLVCLARGFFPDHVELSWWVNGKEVHSGVCTDPQAYK |
| terminal signal peptide) | ESNYSYCLSSRLRVSATFWHNPRNHFRCQVQFHGLSEEDKWPEGSPKPV |
| | TQNISAEAWGRADCGITSASYQQGVLSATILYEILLGKATLYAVLVSTLV |
| | VMAMVKRKNS |
| SEQ ID NO: 99 | ELKVEQNPLFLSMQEGKNYTIYCNYSTTSDRLYWYRQDPGKSLESLFVLL |
| (Cys-substituted, LVL- | SNGAVKQEGRLMASLDTKARLSTLHITAAVHDLSATYFCAVDPTGANSK |
| modified 1913 TCR- | LTFGKGITLSVRPNIQNPEPAVYQLKDPRSQDSTLCLFTDFDSQINVPKTM |
| 41BB α chain predicted | ESGTFITDKCVLDMKAMDSKSNGAIAWSNQTSFTCQDIFKETNATYPSSD |
| sequence without N- | VPCDATLTEKSFETDMNLNFQNLLVIVLRILLLKVAGFNLLMTLRLWSS |
| terminal signal peptide) | |
| SEQ ID NO: 100 | GVTQTPKHLITATGQRVTLRCSPRSGDLSVYWYQQSLDQGLQFLIQYYN |
| (Cys-substituted, LVL- | GEERAKGNILERFSAQQFPDLHSELNLSSLELGDSALYFCASSVAISGEET |
| modified 1913 TCR- | QYFGPGTRLLVLEDLRNVTPPKVSLFEPSKAEIANKQKATLVCLARGFFP |
| 41BB β chain predicted | DHVELSWWVNGKEVHSGVCTDPQAYKESNYSYCLSSRLRVSATFWHNP |
| sequence without N- | RNHFRCQVQFHGLSEEDKWPEGSPKPVTQNISAEAWGRADCGITSASYQ |
| terminal signal peptide) | QGVLSATILYEILLGKATLYAVLVSTLVVMAMVKRKNS |
| SEQ ID NO: 105 | MKSLRVLLVILWLQLSWVWSQQKEVEQNSGPLSVPEGAIASLNCTYSD |
| (Cys-substituted, LVL- | RGSQSFFWYRQYSGKSPELIMFIYSNGDKEDGRFTAQLNKASQYVSLLIR |
| modified 4286 TCR 10-1 | DSQPSDSATYLCAVKSWVIFGKGTRLHILPNIQNPEPAVYQLKDPRSQDS |
| α chain with N-terminal | TLCLFTDFDSQINVPKTMESGTFITDKCVLDMKAMDSKSNGAIAWSNQT |
| signal peptide) | SFTCQDIFKETNATYPSSDVPCDATLTEKSFETDMNLNFQNLLVIVLRILL |
| | LKVAGFNLLMTLRLWSS |
| SEQ ID NO: 106 | MAPGLLCWVLLCLLGAGPVDAGVTQSPTHLIKTRGQQVTLRCSPISGH |
| (Cys-substituted, LVL- | KSVSWYQQVLGQGPQFIFQYYEKEERGRGNFPDRFSARQFPNYSSELNV |
| modified 4286 TCR 10-1 | NALLLGDSALYLCASSPSAQSSYGYTFGSGTRLTVVEDLRNVTPPKVSLF |
| β chain with N-terminal | EPSKAEIANKQKATLVCLARGFFPDHVELSWWVNGKEVHSGVCTDPQA |
| signal peptide) | YKESNYSYCLSSRLRVSATFWHNPRNHFRCQVQFHGLSEEDKWPEGSPK |
| | PVTQNISAEAWGRADCGITSASYQQGVLSATILYEILLGKATLYAVLVST |
| | LVVMAMVKRKNS |
| SEQ ID NO: 107 | QQKEVEQNSGPLSVPEGAIASLNCTYSDRGSQSFFWYRQYSGKSPELIMFI |
| (Cys-substituted, LVL- | YSNGDKEDGRFTAQLNKASQYVSLLIRDSQPSDSATYLCAVKSWVIFGK |
| modified 4286 TCR 10-1 | GTRLHILPNIQNPEPAVYQLKDPRSQDSTLCLFTDFDSQINVPKTMESGTFI |
| α chain predicted | TDKCVLDMKAMDSKSNGAIAWSNQTSFTCQDIFKETNATYPSSDVPCDA |
| sequence without N- | TLTEKSFETDMNLNFQNLLVIVLRILLLKVAGFNLLMTLRLWSS |
| terminal signal peptide) | |
| SEQ ID NO: 108 | GVTQSPTHLIKTRGQQVTLRCSPISGHKSVSWYQQVLGQGPQFIFQYYEK |
| (Cys-substituted, LVL- | EERGRGNFPDRFSARQFPNYSSELNVNALLLGDSALYLCASSPSAQSSYG |
| modified 4286 TCR 10-1 | YTFGSGTRLTVVEDLRNVTPPKVSLFEPSKAEIANKQKATLVCLARGFFP |
| β chain predicted | DHVELSWWVNGKEVHSGVCTDPQAYKESNYSYCLSSRLRVSATFWHNP |
| sequence without N- | RNHFRCQVQFHGLSEEDKWPEGSPKPVTQNISAEAWGRADCGITSASYQ |
| terminal signal peptide) | QGVLSATILYEILLGKATLYAVLVSTLVVMAMVKRKNS |
| SEQ ID NO: 113 | MKSLRVLLVILWLQLSWVWSQQKEVEQNSGPLSVPEGAIASLNCTYSD |
| (Cys-substituted, LVL- | RGSQSFFWYRQYSGKSPELIMFIYSNGDKEDGRFTAQLNKASQYVSLLIR |
| modified 4286 TCR 10-2 | DSQPSDSATYLCAVPNSGYSTLTFGKGTMLLVSPNIQNPEPAVYQLKDPR |
| α chain with N-terminal | SQDSTLCLFTDFDSQINVPKTMESGTFITDKCVLDMKAMDSKSNGAIAWS |
| signal peptide) | NQTSFTCQDIFKETNATYPSSDVPCDATLTEKSFETDMNLNFQNLLVIVL |
| | RILLLKVAGFNLLMTLRLWSS |
| SEQ ID NO: 114 | MAPGLLCWVLLCLLGAGPVDAGVTQSPTHLIKTRGQQVTLRCSPISGH |
| (Cys-substituted, LVL- | KSVSWYQQVLGQGPQFIFQYYEKEERGRGNFPDRFSARQFPNYSSELNV |
| modified 4286 TCR 10-2 | NALLLGDSALYLCASRTGTVTNSYNEQFFGPGTRLTVLEDLRNVTPPKVS |
| β chain with N-terminal | LFEPSKAEIANKQKATLVCLARGFFPDHVELSWWVNGKEVHSGVCTDPQ |
| signal peptide) | AYKESNYSYCLSSRLRVSATFWHNPRNHFRCQVQFHGLSEEDKWPEGSP |
| | KPVTQNISAEAWGRADCGITSASYQQGVLSATILYEILLGKATLYAVLVS |
| | TLVVMAMVKRKNS |
| SEQ ID NO: 115 | QQKEVEQNSGPLSVPEGAIASLNCTYSDRGSQSFFWYRQYSGKSPELIMFI |
| (Cys-substituted, LVL- | YSNGDKEDGRFTAQLNKASQYVSLLIRDSQPSDSATYLCAVPNSGYSTLT |
| modified 4286 TCR 10-2 | FGKGTMLLVSPNIQNPEPAVYQLKDPRSQDSTLCLFTDFDSQINVPKTME |
| α chain predicted | SGTFITDKCVLDMKAMDSKSNGAIAWSNQTSFTCQDIFKETNATYPSSDV |
| sequence without N- | PCDATLTEKSFETDMNLNFQNLLVIVLRILLLKVAGFNLLMTLRLWSS |
| terminal signal peptide) | |
| SEQ ID NO: 116 | GVTQSPTHLIKTRGQQVTLRCSPISGHKSVSWYQQVLGQGPQFIFQYYEK |
| (Cys-substituted, LVL- | EERGRGNFPDRFSARQFPNYSSELNVNALLLGDSALYLCASRTGTVTNSY |
| modified 4286 TCR 10-2 | NEQFFGPGTRLTVLEDLRNVTPPKVSLFEPSKAEIANKQKATLVCLARGF |
| β chain predicted | FPDHVELSWWVNGKEVHSGVCTDPQAYKESNYSYCLSSRLRVSATFWH |
| sequence without N- | NPRNHFRCQVQFHGLSEEDKWPEGSPKPVTQNISAEAWGRADCGITSAS |
| terminal signal peptide) | YQQGVLSATILYEILLGKATLYAVLVSTLVVMAMVKRKNS |
| SEQ ID NO: 121 | MLLLLIPVLGMIFALRDARAQSVSQHNHHVILSEAASLELGCNYSYGGT |
| (Cys-substituted, LVL- | VNLFWYVQYPGQHLQLLLKYFSGDPLVKGIKGFEAEFIKSKFSFNLRKPS |
| modified 4286 TCR 10-3 | VQWSDTAEYFCAVKDTGFQKLVFGTGTRLLVSPNIQNPEPAVYQLKDPR |
| α chain with N-terminal | SQDSTLCLFTDFDSQINVPKTMESGTFITDKCVLDMKAMDSKSNGAIAWS |
| signal peptide) | NQTSFTCQDIFKETNATYPSSDVPCDATLTEKSFETDMNLNFQNLLVIVL |
| | RILLLKVAGFNLLMTLRLWSS |
| SEQ ID NO: 122 | MLLLLLLLGPGISLLLPGSLAGSGLGAVVSQHPSWVICKSGTSVKIECRS |
| (Cys-substituted, LVL- | LDFQATTMFWYRQFPKQSLMLMATSNEGSKATYEQGVEKDKFLINHAS |
| modified 4286 TCR 10-3 | LTLSTLTVTSAHPEDSSFYICSARGGRNSFEQFFGPGTRLTVLEDLRNVTPP |
| β chain with N-terminal | KVSLFEPSKAEIANKQKATLVCLARGFFPDHVELSWWVNGKEVHSGVCT |
| signal peptide) | DPQAYKESNYSYCLSSRLRVSATFWHNPRNHFRCQVQFHGLSEEDKWPE |
| | GSPKPVTQNISAEAWGRADCGITSASYQQGVLSATILYEILLGKATLYAV |
| | LVSTLVVMAMVKRKNS |
| SEQ ID NO: 123 | QSVSQHNHHVILSEAASLELGCNYSYGGTVNLFWYVQYPGQHLQLLLKY |
| (Cys-substituted, LVL- | FSGDPLVKGIKGFEAEFIKSKFSFNLRKPSVQWSDTAEYFCAVKDTGFQK |
| modified 4286 TCR 10-3 | LVFGTGTRLLVSPNIQNPEPAVYQLKDPRSQDSTLCLFTDFDSQINVPKTM |
| α chain predicted | ESGTFITDKCVLDMKAMDSKSNGAIAWSNQTSFTCQDIFKETNATYPSSD |
| sequence without N- | VPCDATLTEKSFETDMNLNFQNLLVIVLRILLLKVAGFNLLMTLRLWSS |
| terminal signal peptide) | |
| SEQ ID NO: 129 | GSGLGAVVSQHPSWVICKSGTSVKIECRSLDFQATTMFWYRQFPKQSLM |
| (Cys-substituted, LVL- | LMATSNEGSKATYEQGVEKDKFLINHASLTLSTLTVTSAHPEDSSFYICSA |
| modified 4286 TCR 10-3 | RGGRNSFEQFFGPGTRLTVLEDLRNVTPPKVSLFEPSKAEIANKQKATLV |
| β chain predicted | CLARGFFPDHVELSWWVNGKEVHSGVCTDPQAYKESNYSYCLSSRLRV |
| sequence without N- | SATFWHNPRNHFRCQVQFHGLSEEDKWPEGSPKPVTQNISAEAWGRADC |
| terminal signal peptide) | GITSASYQQGVLSATILYEILLGKATLYAVLVSTLVVMAMVKRKNS |
| SEQ ID NO: 130 | MLLLLIPVLGMIFALRDARAQSVSQHNHHVILSEAASLELGCNYSYGGT |
| (Cys-substituted, LVL- | VNLFWYVQYPGQHLQLLLKYFSGDPLVKGIKGFEAEFIKSKFSFNLRKPS |
| modified 4286 TCR 10-4 | VQWSDTAEYFCAVKDTGFQKLVFGTGTRLLVSPNIQNPEPAVYQLKDPR |
| α chain with N-terminal | SQDSTLCLFTDFDSQINVPKTMESGTFITDKCVLDMKAMDSKSNGAIAWS |
| signal peptide) | NQTSFTCQDIFKETNATYPSSDVPCDATLTEKSFETDMNLNFQNLLVIVL |
| | RILLLKVAGFNLLMTLRLWSS |
| SEQ ID NO: 131 | MLLLLLLLGPGISLLLPGSLAGSGLGAVVSQHPSWVICKSGTSVKIECRS |
| (Cys-substituted, LVL- | LDFQATTMFWYRQFPKQSLMLMATSNEGSKATYEQGVEKDKFLINHAS |
| modified 4286 TCR 10-4 | LTLSTLTVTSAHPEDSSFYICSARGGARSYEQYFGPGTRLTVTEDLRNVTP |
| β chain with N-terminal | PKVSLFEPSKAEIANKQKATLVCLARGFFPDHVELSWWVNGKEVHSGVC |
| signal peptide) | TDPQAYKESNYSYCLSSRLRVSATFWHNPRNHFRCQVQFHGLSEEDKWP |
| | EGSPKPVTQNISAEAWGRADCGITSASYQQGVLSATILYEILLGKATLYA |
| | VLVSTLVVMAMVKRKNS |
| SEQ ID NO: 132 | QSVSQHNHHVILSEAASLELGCNYSYGGTVNLFWYVQYPGQHLQLLLKY |
| (Cys-substituted, LVL- | FSGDPLVKGIKGFEAEFIKSKFSFNLRKPSVQWSDTAEYFCAVKDTGFQK |
| modified 4286 TCR 10-4 | LVFGTGTRLLVSPNIQNPEPAVYQLKDPRSQDSTLCLFTDFDSQINVPKTM |
| α chain predicted | ESGTFITDKCVLDMKAMDSKSNGAIAWSNQTSFTCQDIFKETNATYPSSD |
| sequence without N- | VPCDATLTEKSFETDMNLNFQNLLVIVLRILLLKVAGFNLLMTLRLWSS |
| terminal signal peptide) | |
| SEQ ID NO: 124 | GSGLGAVVSQHPSWVICKSGTSVKIECRSLDFQATTMFWYRQFPKQSLM |
| (Cys-substituted, LVL- | LMATSNEGSKATYEQGVEKDKFLINHASLTLSTLTVTSAHPEDSSFYICSA |
| modified 4286 TCR 10-4 | RGGARSYEQYFGPGTRLTVTEDLRNVTPPKVSLFEPSKAEIANKQKATLV |
| B chain predicted | CLARGFFPDHVELSWWVNGKEVHSGVCTDPQAYKESNYSYCLSSRLRV |
| sequence without N- | SATFWHNPRNHFRCQVQFHGLSEEDKWPEGSPKPVTQNISAEAWGRADC |
| terminal signal peptide) | GITSASYQQGVLSATILYEILLGKATLYAVLVSTLVVMAMVKRKNS |

Nucleotide sequences encoding the variable regions of the α and β chains of the TCRs of Table 9 were independently cloned into MSGV1-based retroviral vectors with the following expression cassette configuration; 5′Ncol-VDJα-mCα-Furin/SerGly/P2A-VJa-mCa-EcoRI3′.

The TCRB and TCRα chains were separated by a Furin Ser/Gly P2A linker peptide (SEQ ID NO: 133). Without being bound to a particular theory or mechanism, it is believed that the linker peptide provides comparable expression efficiency of the two chains (Szymczak et al., Nat. Biotechnol., 22 (5); 589-94 (2004)).

The TCR expression cassette of the retroviral vector encoded, from 5′ to 3′, the TCRB and TCRα chains separated by the linker peptide. The amino acid sequence encoded by the TCR expression cassette for each respective TCR is shown in Table 10. In Table 10, the CDRs are underlined, the constant regions are italicized, and the linker peptide is shown in bold.

TABLE 10

| TCR Name | Amino acid sequence encoded by TCR Expression Cassette |
|---|---|
| 3333 TCR-A | MASLLFFCGAFYLLGTGSMDADVTQTPRNRITKTGKRIMLECSQTKGHDRMYW |
| | YRQDPGLGLRLIYYSFDVKDINKGEISDGYSVSRQAQAKFSLSLESAIPNQTALYF |
| | CATSDFRGKPQHFGDGTRLSILEDLRNVTPPKVSLFEPSKAELANKQKATLVCLARGF |
| | FPDHVELSWWVNGKEVHSGVCTDPQAYKESNYSYCLSSRLRVSATFWHNPRNHFRCQ |
| | VQFHGLSEEDKWPEGSPKPVTQNISAEAWGRADCGITSASYQQGVLSATILYEILLGKA |
| | TLYAVLVSTLVVMAMVKRKNSRAKRSGSGATNFSLLKQAGDVEENPGPMMKSLR |
| | VLLVILWLQLSWVWSQQKEVEQNSGPLSVPEGAIASLNCTYSDRGSQSFFWYRQ |
| | YSGKSPELIMSIYSNGDKEDGRFTAQLNKASQYVSLLIRDSQPSDSATYLCAVNT |
| | ASGTYKYIFGTGTRLKVLANIQNPEPAVYQLKDPRSQDSTLCLFTDFDSQINVPKTM |
| | ESGTFITDKCVLDMKAMDSKSNGAIAWSNQTSFTCQDIFKETNATYPSSDVPCDATLT |
| | EKSFETDMNLNFQNLLVIVLRILLLKVAGENLLMTLRLWSS (SEQ ID NO: 134) |
| 3333 TCR-C | MAIRLLCYMGFYFLGAGLMEADIYQTPRYLVIGTGKKITLECSQTMGHDKMYW |
| | YQQDPGMELHLIHYSYGVNSTEKGDLSSESTVSRIRTEHFPLTLESARPSHTSQYL |
| | CASSRPGSEKLFFGSGTQLSVLEDLRNVTPPKVSLFEPSKAELANKQKATLVCLARGF |
| | FPDHVELSWWVNGKEVHSGVCTDPQAYKESNYSYCLSSRLRVSATFWHNPRNHFRCQ |
| | VQFHGLSEEDKWPEGSPKPVTQNISAEAWGRADCGITSASYQQGVLSATILYEILLGKA |
| | TLYAVLVSTLVVMAMVKRKNSRAKRSGSGATNFSLLKQAGDVEENPGPMMKSLR |
| | VLLVILWLQLSWVWSQQKEVEQNSGPLSVPEGAIASLNCTYSDRGSQSFFWYRQ |
| | YSGKSPELIMSIYSNGDKEDGRFTAQLNKASQYVSLLIRDSQPSDSATYLCAVNR |
| | AREAAGNKLTFGGGTRVLVKPNIQNPEPAVYQLKDPRSQDSTLCLFTDFDSQINVP |
| | KTMESGTFITDKCVLDMKAMDSKSNGALAWSNQTSFTCQDIFKETNATYPSSDVPCDA |
| | TLTEKSFETDMNLNFQNLLVIVLRILLLKVAGENLLMTLRLWSS (SEQ ID NO: 135) |
| 1913 TCR-41BB | MAFRLLCCVAFCLLGAGPVDSGVTQTPKHLITATGQRVTLRCSPRSGDLSVYWY |
| | QQSLDQGLQFLIQYYNGEERAKGNILERFSAQQFPDLHSELNLSSLELGDSALYFC |
| | ASSVAISGEETQYFGPGTRLLVLEDLRNVTPPKVSLFEPSKAELANKQKATLVCLARG |
| | FFPDHVELSWWVNGKEVHSGVCTDPQAYKESNYSYCLSSRLRVSATFWHNPRNHFRC |
| | QVQFHGLSEEDKWPEGSPKPVTQNISAEAWGRADCGITSASYQQGVLSATILYEILLGK |
| | ATLYAVLVSTLVVMAMVKRKNSRAKRSGSGATNFSLLKQAGDVEENPGPMKKLL |
| | AMILWLQLDRLSGELKVEQNPLFLSMQEGKNYTIYCNYSTTSDRLYWYRQDPG |
| | KSLESLFVLLSNGAVKQEGRLMASLDTKARLSTLHITAAVHDLSATYFCAVDPT |
| | GANSKLTFGKGITLSVRPNIQNPEPAVYQLKDPRSQDSTLCLFTDFDSQINVPKTMES |
| | GTFITDKCVLDMKAMDSKSNGAIAWSNQTSFTCQDIFKETNATYPSSDVPCDATLTEK |
| | SFETDMNLNFQNLLVIVLRILLLKVAGENLLMTLRLWSS (SEQ ID NO: 136) |
| 4286 TCR 10-1 | MAPGLLCWVLLCLLGAGPVDAGVTQSPTHLIKTRGQQVTLRCSPISGHKS |
| | VSWYQQVLGQGPQFIFQYYEKEERGRGNFPDRFSARQFPNYSSELNVNALLL |
| | GDSALYLCASSPSAQSSYGYTFGSGTRLTVVEDLRNITPPKVSLFEPSKAELAN |
| | KQKATLVCLARGFFPDHVELSWWVNGKEVHSGVCTDPQAYKESNYSYCLSSRLR |
| | VSATFWHNPRNHFRCQVQFHGLSEEDKWPEGSPKPVTQNISAEAWGRADCGITS |
| | ASYQQGVLSATILYEILLGKATLYAVLVSTLVVMAMVKRKNSRAKRSGSGATNFS |
| | LLKQAGDVEENPGMKSLRVLLVILWLQLSWVWSQQKEVEQNSGPLSVPEG |
| | AIASLNCTYSDRGSQSFFWYRQYSGKSPELIMFIYSNGDKEDGRFTAQLNKA |
| | SQYVSLLIRDSQPSDSATYLCAVKSWVIFGKGTRLHILPNIQNPEPAVYQLKDP |
| | RSQDSTLCLFTDFDSQINVPKTMESGTFITDKCVLDMKAMDSKSNGAIAWSNQTS |
| | FTCQDIFKETNATYPSSDVPCDATLTEKSFETDMNLNFQNLLVIVLRILLLKVAGF |
| | NLLMTLRLWSS (SEQ ID NO: 137) |
| 4286 TCR 10-2 | MAPGLLCWVLLCLLGAGPVDAGVTQSPTHLIKTRGQQVTLRCSPISGHKS |
| | VSWYQQVLGQGPQFIFQYYEKEERGRGNFPDRFSARQFPNYSSELNVNALLL |
| | GDSALYLCASRTGTVTNSYNEQFFGPGTRLTVLEDLRNVTPPKVSLFEPSKAEI |
| | ANKQKATLVCLARGFFPDHVELSWWVNGKEVHSGVCTDPQAYKESNYSYCLSSR |
| | LRVSATFWHNPRNHFRCQVQFHGLSEEDKWPEGSPKPVTQNISAEAWGRADCGI |
| | TSASYQQGVLSATILYEILLGKATLYAVLVSTLVVMAMVKRKNSRAKRSGSGATN |
| | FSLLKQAGDVEENPGMKSLRVLLVILWLQLSWVWSQQKEVEQNSGPLSVP |
| | EGAIASLNCTYSDRGSQSFFWYRQYSGKSPELIMFIYSNGDKEDGRFTAQLN |
| | KASQYVSLLIRDSQPSDSATYLCAVPNSGYSTLTFGKGTMLLVSPNIQNPEPA |
| | VYQLKDPRSQDSTLCLFTDFDSQINVPKTMESGTFITDKCVLDMKAMDSKSNGAI |
| | AWSNQTSFTCQDIFKETNATYPSSDVPCDATLTEKSFETDMNLNFQNLLVIVLRIL |
| | LLKVAGFNLLMTLRLWSS (SEQ ID NO: 138) |
| 4286 TCR 10-3 | MLLLLLLLGPGISLLLPGSLAGSGLGAVVSQHPSWVICKSGTSVKIECRSLDF |
| | QATTMFWYRQFPKQSLMLMATSNEGSKATYEQGVEKDKFLINHASLTLSTL |
| | TVTSAHPEDSSFYICSARGGRNSFEQFFGPGTRLTVLEDLRNVTPPKVSLFEPSK |
| | AEIANKQKATLVCLARGFFPDHVELSWWVNGKEVHSGVCTDPQAYKESNYSYCLS |
| | SRLRVSATFWHNPRNHFRCQVQFHGLSEEDKWPEGSPKPITQNISAEAWGRADC |
| | GITSASYQQGVLSATILYEILLGKATLYAVLVSTLVVMAMVKRKNSRAKRSGSGAT |
| | NFSLLKQAGDVEENPGMLLLLIPVLGMIFALRDARAQSVSQHNHHVILSEA |
| | ASLELGCNYSYGGTVNLFWYVQYPGQHLQLLLKYFSGDPLVKGIKGFEAEFI |
| | KSKFSFNLRKPSVQWSDTAEYFCAVKDTGFQKLVFGTGTRLLVSPNIQNPEP |
| | AVYQLKDPRSQDSTLCLFTDFDSQINVPKTMESGTFITDKCVLDMKAMDSKSNG |
| | AIAWSNQTSFTCQDIFKETNATYPSSDVPCDATLTEKSFETDMNLNFQNLLVIVLRI |
| | LLLKVAGFNLLMTLRLWSS (SEQ ID NO: 139) |
| 4286 TCR 10-4 | MLLLLLLLGPGISLLLPGSLAGSGLGAVVSQHPSWVICKSGTSVKIECRSLDF |
| | QATTMFWYRQFPKQSLMLMATSNEGSKATYEQGVEKDKFLINHASLTLSTL |
| | TVTSAHPEDSSFYICSARGGARSYEQYEGPGTRLTVTEDLRNVTPPKVSLFEPS |
| | KAELANKQKATLVCLARGFFPDHVELSWWVNGKEVHSGVCTDPQAYKESNYSYC |
| | LSSRLRVSATFWHNPRNHFRCQVQFHGLSEEDKWPEGSPKPVTQNISAEAWGRA |
| | DCGITSASYQQGVLSATILYEILLGKATLYAVLVSTLVVMAMVKRKNSRAKRSGSG |
| | ATNFSLLKQAGDVEENPGMLLLLIPVLGMIFALRDARAQSVSQHNHHVILS |
| | EAASLELGCNYSYGGTVNLFWYVQYPGQHLQLLLKYFSGDPLVKGIKGFEA |
| | EFIKSKFSFNLRKPSVQWSDTAEYFCAVKDTGFQKLVFGTGTRLLVSPNIQNP |
| | EPAVYQLKDPRSQDSTLCLFTDFDSQINVPKTMESGTFITDKCVLDMKAMDSKSN |
| | GAIAWSNQTSFTCQDIFKETNATYPSSDVPCDATLTEKSFETDMNLNFQNLLVIVL |
| | RILLLKVAGFNLLMTLRLWSS (SEQ ID NO: 140) |

### EXAMPLE 5

This example demonstrates that 3333 TCR-A and 3333 TCR-C recognize the CDKN2A frameshift mutated peptide AVCPWTWLR (SEQ ID NO: 141).

Healthy donor PBL were independently transduced with a retroviral vector encoding the 3333 TCR-A and 3333 TCR-C expression cassette of Table 10 or a corresponding expression cassette encoding the 3333 TCR-B and 3333 TCR-D of Example 1 to produce effector cells.

The target cells were HLA-A*03; 01+. HLA-A*11; 01+dendritic cells (DCs) that were (i) transfected with P16INK4a RNA encoding mutated peptide AVCPWTWLR (SEQ ID NO: 141), (ii) transfected with RNA encoding WT P16INK4a, or (iii) pulsed with mutated peptide mutated peptide AVCPWTWLR (SEQ ID NO: 141).

The effector cells were co-cultured with the target cells. The levels of interferon gamma (IFN-γ) secreted were measured by enzyme-linked immunosorbent spot (ELISpot) assay. The ELISpot results showed that two TCRs from Patient 3333 specifically recognized both pulsed and transfected mutated peptide AVCPWTWLR (SEQ ID NO: 141), namely 3333 TCR-A and 3333 TCR-C. These two TCRs did not recognize WT peptide. The ELISpot results also showed that mock-transduced PBL (control), 3333 TCR-α-and 3333 TCR D-transduced PBL did not recognize mutated peptide AVCPWTWLR (SEQ ID NO: 141). Effector cells treated with phorbol myristate acetate (PMA) (positive control) secreted IFN-γ. Effector cells cultured alone (without target cells) (negative control) and target cells treated with dimethyl sulfoxide (DMSO) (negative control) showed no detectable IFN-γ secretion.

The percentage of activated TCR-transduced CD8+ T cells upregulating 4-1BB expression was measured by flow cytometry. The results are shown in Table 11. The flow cytometry results showed that two TCRs from Patient 3333 specifically recognized mutated peptide AVCPWTWLR (SEQ ID NO: 141), namely 3333 TCR-A and 3333 TCR-C. The flow cytometry results also showed that 3333 TCR-α- and 3333 TCR D-transduced PBL did not recognize mutated peptide AVCPWTWLR (SEQ ID NO: 141).

TABLE 11
Gated through live > CD8+ > mTCR+ cells

| | % 4-1BB | | | |
|---|---|---|---|---|
| | 3333 | 3333 | 3333 | 3333 |
| | TCR-A | TCR-B | TCR-C | TCR-D |
| Mutant CDKN2A RNA | 41.0 | 7.72 | 45.7 | 0.85 |
| WT CDKN2A RNA | 1.46 | 7.32 | 1.75 | 0.78 |
| Mutant CDKN2A peptide | 24.0 | 12.8 | 45.8 | 2.36 |
| TCRs alone | 0.39 | 0.32 | 1.10 | 0.54 |
| PMA | 24.2 | 36.2 | 48.6 | 56.2 |

### EXAMPLE 6

This example demonstrates that 1913 TCR-41BB recognizes the CDKN2A frameshift mutated peptide AVCPWTWLR (SEQ ID NO: 141).

Healthy donor PBL were transduced with a retroviral vector encoding the 1913 TCR-41BB expression cassette of Table 10 to produce effector cells. The target cells were HLA-A*03; 01+, HLA-A*11; 01+DCs that were (i) transfected with P16INK4a RNA encoding mutated peptide AVCPWTWLR (SEQ ID NO: 141), (ii) transfected with RNA encoding WT P16INK4a, (iii) transfected with P14ARF RNA encoding mutated peptide AVCPWTWLR (SEQ ID NO: 141). (iv) transfected with RNA encoding WT P14ARF, or (v) pulsed with mutated peptide AVCPWTWLR (SEQ ID NO: 141).

The effector cells were co-cultured with the target cells. The levels of IFN-γ secreted were measured by ELISpot assay. The ELISpot results showed that the 1913 TCR-41BB specifically recognized transfected or pulsed mutated peptide AVCPWTWLR (SEQ ID NO: 141), but did not recognize WT P16INK4a or WT P14ARF. The ELISpot results also showed that mock-transduced PBL (control) did not recognize AVCPWTWLR (SEQ ID NO: 141). Effector cells treated with PMA (positive control) upregulated secreted IFN-γ. Effector cells cultured alone (without target cells) (negative control) showed no detectable IFN-γ secretion.

The percentage of activated TCR-transduced CD8+ T cells upregulating 4-1BB expression was measured by flow cytometry. The results are shown in Table 12. The flow cytometry results showed that the 1913 TCR-41BB specifically recognized AVCPWTWLR (SEQ ID NO: 141).

TABLE 12
Gated through live > CD8+ > mTCR+ cells

| | % 4-1BB |
|---|---|
| P16INK4a Mutant RNA | 69.9 |
| P16INK4a WT RNA | 3.76 |
| P14ARF Mutant RNA | 52.8 |
| P14ARF WT RNA | 2.53 |
| AVCPWTWLR (SEQ ID NO: 141) | 67.6 |
| Mutant peptide | |
| TCR | 2.97 |
| PMA | 85.7 |

### EXAMPLE 7

This example demonstrates that 3333 TCR-A and 3333 TCR-C recognize the CDKN2A frameshift mutated peptide AVCPWTWLR (SEQ ID NO: 141) presented by a HLA-A*03; 01 molecule. This example also demonstrates that 1913 TCR-41BB recognizes the CDKN2A frameshift mutated peptide AVCPWTWLR (SEQ ID NO: 141) presented by a HLA-A*11; 01 molecule.

Healthy donor PBL were independently transduced with the retroviral vector encoding the 3333 TCR-A, 3333 TCR-C, or 1913 TCR-41BB expression cassette of Table 10 to produce effector cells. Target cells were Cos7 cells independently transfected with HLA-A*03; 01 or HLA-A*11; 01 and pulsed with mutated peptide AVCPWTWLR (SEQ ID NO: 141).

The effector cells were co-cultured with the target cells. The levels of IFN-γ secreted were measured by ELISpot assay. The ELISpot results showed that 3333 TCR-A and 3333 TCR-C recognized the CDKN2A frameshift mutated peptide AVCPWTWLR (SEQ ID NO: 141) presented by a HLA-A*03; 01 molecule. The ELISpot results also showed that 1913 TCR-41BB recognized the CDKN2A frameshift mutated peptide AVCPWTWLR (SEQ ID NO: 141) presented by an HLA-A*11; 01 molecule. Effector cells treated with PMA (positive control) secreted IFN-γ. Effector cells cultured alone (without target cells) (negative control) showed no detectable IFN-γ secretion.

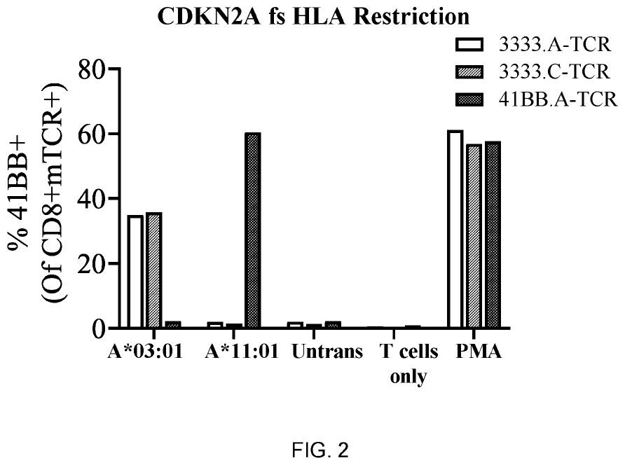

The percentage of activated TCR-transduced CD8+ T cells upregulating 4-1BB expression was measured by flow cytometry. The results are shown in FIG. 2. The flow cytometry results showed that 3333 TCR-A and 3333 TCR-C recognized the CDKN2A frameshift mutated peptide AVCPWTWLR (SEQ ID NO: 141) presented by a HLA-A*03; 01 molecule. The flow cytometry results also showed that 1913 TCR-41BB recognized the CDKN2A frameshift mutated peptide AVCPWTWLR (SEQ ID NO: 141) presented by a HLA-A*11; 01 molecule. Effector cells treated with PMA (positive control) upregulated 4-1BB expression. Effector cells cultured alone (without target cells) (negative control) and untransduced PBL co-cultured with target cells (negative control) showed no detectable 4-1BB upregulation.

### EXAMPLE 8

This example demonstrates that 3333 TCR-A, 3333 TCR-C, and 1913 TCR-41BB sensitively and specifically recognize target cells transfected with CDKN2A frameshift RNA or pulsed with CDKN2A frameshift mutant peptide.

Healthy donor PBL were independently transduced with the retroviral vector encoding the 3333 TCR-A, 3333 TCR-C, or 1913 TCR-41BB expression cassette of Table 10 to produce effector cells.

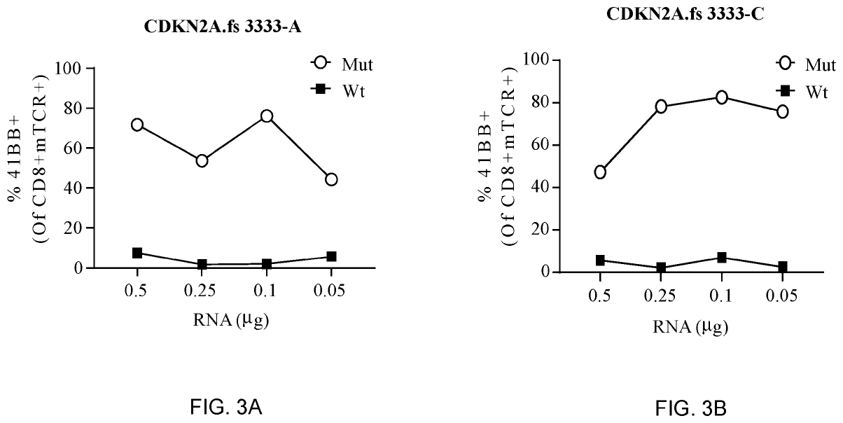

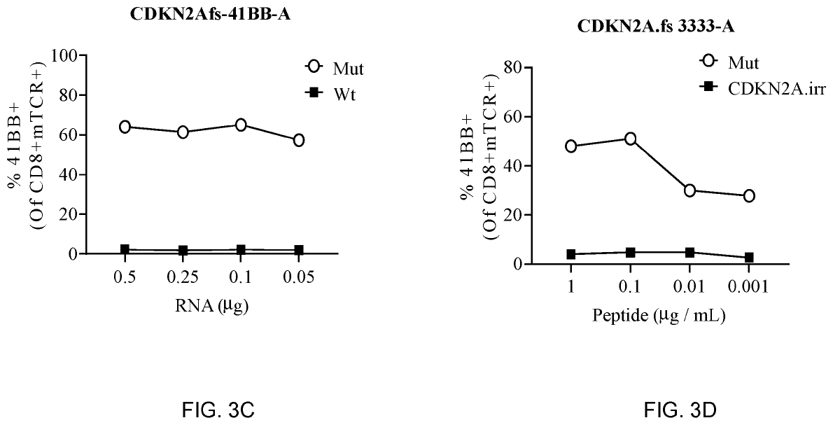

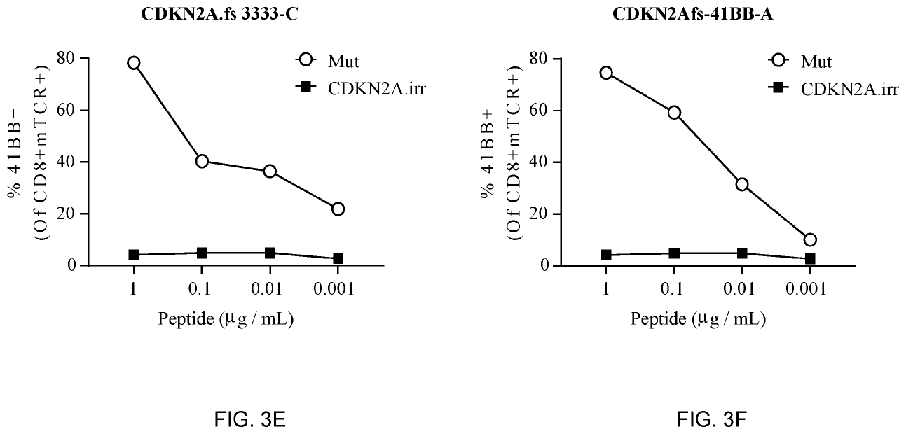

The target cells were HLA-A*03; 01+, HLA-A*11; 01+DCs that were (i) transfected with P16INK4a RNA encoding mutated peptide AVCPWTWLR (SEQ ID NO: 141) at the amounts shown in FIGS. 3A-3C. (ii) transfected with RNA encoding WT P16INK4a at the amounts shown in FIGS. 3A-3C, (iii) pulsed with mutated peptide AVCPWTWLR (SEQ ID NO: 141) at the concentrations shown in FIGS. 3D-3F, or (iv) pulsed with an irrelevant CDKN2A peptide at the concentrations shown in FIGS. 3D-3F.

The effector cells were co-cultured with the target cells. The percentage of activated TCR-transduced CD8+ T cells upregulating 4-1BB expression was measured by flow cytometry. The results are shown in FIGS. 3A-3F. The results showed that 3333 TCR-A, 3333 TCR-C, and 1913 TCR-41BB sensitively and specifically recognized target cells transfected with CDKN2A frameshift RNA or pulsed with CDKN2A frameshift mutant peptide.

### EXAMPLE 9

This example demonstrates that 3333 TCR-A and 3333 TCR-C specifically recognize an autologous tumor line expressing CDKN2A frameshift mutated peptide AVCPWTWLR (SEQ ID NO: 141) and HLA-A*03; 01. This example also demonstrates that 1913 TCR-41BB specifically recognizes an autologous tumor line expressing CDKN2A frameshift mutated peptide AVCPWTWLR (SEQ ID NO: 141) and HLA-A*11; 01.

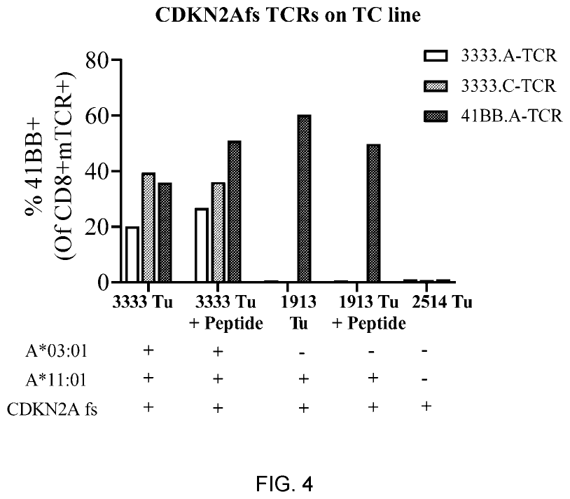

Healthy donor PBL were independently transduced with the retroviral vector encoding the 3333 TCR-A, 3333 TCR-C, or 1913 TCR-41BB expression cassette of Table 10 to produce effector cells. Target cells were (i) autologous tumor cell line from Patient 3333, (ii) autologous tumor cell line from Patient 3333 pulsed with CDKN2A frameshift mutated peptide AVCPWTWLR (SEQ ID NO: 141), (iii) autologous tumor cell line from Patient 1913. (iv) autologous tumor cell line from Patient 1913 pulsed with CDKN2A frameshift mutated peptide AVCPWTWLR (SEQ ID NO: 141), (v) autologous tumor cell line from Patient 2514, or (vi) autologous tumor cell line from Patient 2514 pulsed with CDKN2A frameshift mutated peptide AVCPWTWLR (SEQ ID NO: 141). HLA-A*11; 01 and HLA-A*3; 01 expression by the tumor cell lines is shown in FIG. 4.

The effector cells were co-cultured with the target cells. The levels of IFN-γ secreted were measured by ELISpot assay. The ELISpot results showed that 3333 TCR-A and 3333 TCR-C specifically recognized the tumor cell line expressing the CDKN2A frameshift mutated peptide AVCPWTWLR (SEQ ID NO: 141) and HLA-A*03; 01. The ELISpot results also showed that 1913 TCR-41BB specifically recognized (i) the tumor cell line expressing the CDKN2A frameshift mutated peptide AVCPWTWLR (SEQ ID NO: 141) and HLA-A*11; 01 and (ii) the tumor cell line expressing the CDKN2A frameshift mutated peptide AVCPWTWLR (SEQ ID NO: 141) and HLA-A*3; 01. No detectable IFN-γ secretion was observed following co-culture of effector cells with the autologous tumor cell line from Patient 2514, with or without pulse with CDKN2A frameshift mutated peptide AVCPWTWLR (SEQ ID NO: 141).

The percentage of activated TCR-transduced CD8+ T cells upregulating 4-1BB expression was measured by flow cytometry. The results are shown in FIG. 4. The flow cytometry results showed that 3333 TCR-A and 3333 TCR-C specifically recognized the autologous tumor line expressing the CDKN2A frameshift mutated peptide AVCPWTWLR (SEQ ID NO: 141) and HLA-A*03; 01. The flow cytometry results also showed that 1913 TCR-41BB specifically recognized (i) the autologous tumor line expressing the CDKN2A frameshift mutated peptide AVCPWTWLR (SEQ ID NO: 141) and HLA-A*11; 01 and (ii) the tumor cell line expressing the CDKN2A frameshift mutated peptide AVCPWTWLR (SEQ ID NO: 141) and HLA-A*3; 01. No detectable 4-1BB upregulation was observed following co-culture of autologous tumor from Patient 2514 with effector cells.

### EXAMPLE 10

This example demonstrates that 4286 TCR 10-1, 4286 TCR 10-2, 4286 TCR 10-3, and 4286 TCR 10-4 bind to 9-mer LLVDLAEEL (SEQ ID NO: 142) tetramer and 10-mer RLLVDLAEEL (SEQ ID NO: 143).

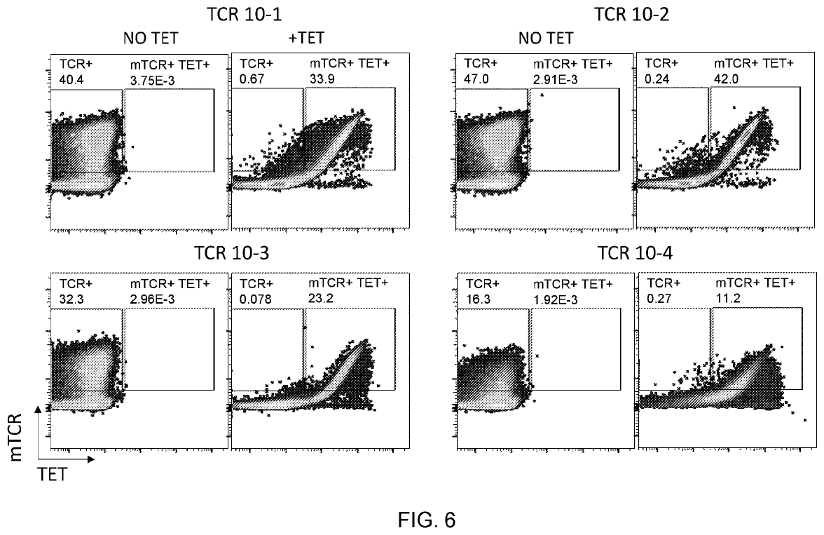

Healthy donor PBL were independently transduced with a retroviral vector encoding the 4286 TCR 10-1, 4286 TCR 10-2, 4286 TCR 10-3, or 4286 TCR 10-4 expression cassette of Table 10. The transduced cells were stained with 9-mer LLVDLAEEL (SEQ ID NO: 142) tetramer and 10-mer RLLVDLAEEL (SEQ ID NO: 143) as well as an antibody that binds to the murine TCR constant region (mTCR) to detect transgene TCR expression in the transduced cells. The results are shown in FIG. 6. The results showed that the transduced cells bound to 9-mer LLVDLAEEL (SEQ ID NO: 142) tetramer and 10-mer RLLVDLAEEL (SEQ ID NO: 143).

### EXAMPLE 11

This example demonstrates that 4286 TCR 10-1, 4286 TCR 10-2, 4286 TCR 10-3, and 4286 TCR 10-4 provide avid and specific recognition of mutated 9-mer LLVDLAEEL (SEQ ID NO: 142) peptide and mutated 10-mer RLLVDLAEEL (SEQ ID NO: 143) peptide.

Healthy donor PBL were independently transduced with a retroviral vector encoding the 4286 TCR 10-1, 4286 TCR 10-2, 4286 TCR 10-3, or 4286 TCR 10-4 expression cassette of Table 10 to produce effector cells.

Target cells were HLA-A*2; 01+dendritic cells pulsed with various concentrations of mutated 9-mer LLVDLAEEL peptide (SEQ ID NO: 142), mutated 10-mer RLLVDLAEEL peptide (SEQ ID NO: 143). WT 9-mer LPVDLAEEL peptide (SEQ ID NO: 144), or WT 10-mer RLPVDLAEEL peptide (SEQ ID NO: 145).

Effector cells were co-cultured with target cells. IFN-γ secretion was measured by ELISpot per 2e4 effector cells. The results are shown in Table 13.

TABLE 13

| | lowest concentration of | | lowest concentration of | |
|---|---|---|---|---|
| | mutated 9-mer peptide | | mutated 10-mer peptide | |
| TCR | showing reactivity | | showing reactivity | |
| 4286 TCR 10-1 | 100 | pg/mL | 100 | pg/mL |
| 4286 TCR 10-2 | 100 | pg/mL | 100 | pg/mL |
| 4286 TCR 10-3 | 1 | ng/mL | 1 | ng/mL |
| 4286 TCR 10-4 | 1 | μg/mL | 1 | μg/mL |

None of the four TCRs recognized target cells pulsed with the corresponding WT peptide. Effector cells treated with PMA (positive control) secreted IFN-γ. Effector cells cultured alone (without target cells) (negative control) showed no detectable IFN-γ secretion.

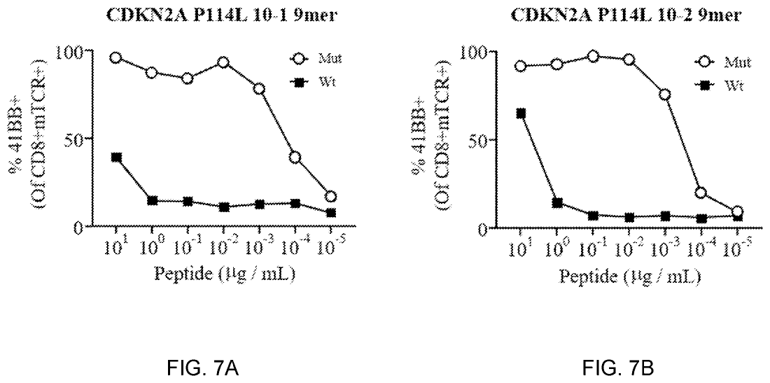

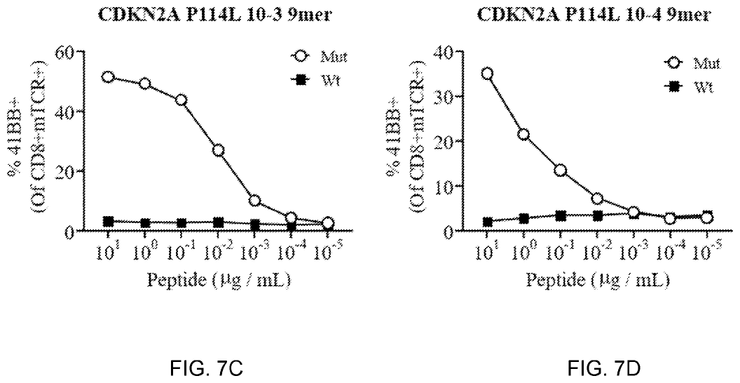

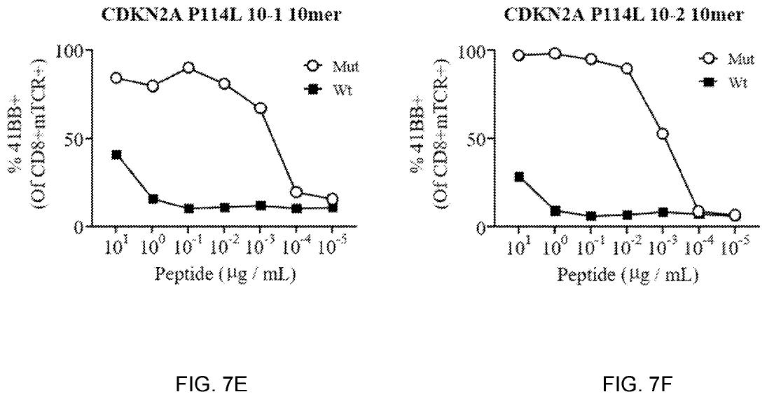

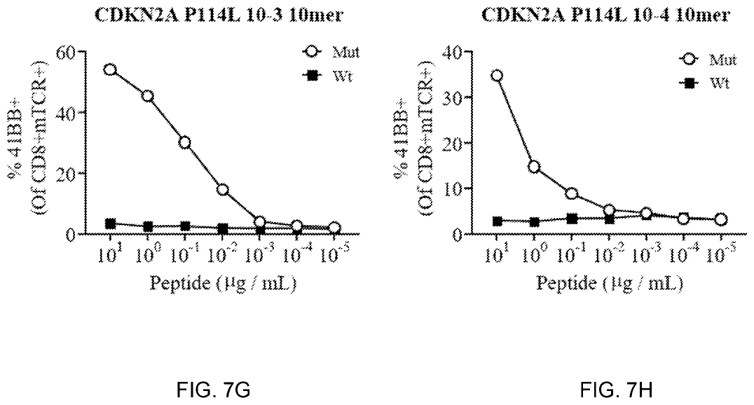

The percentage of activated TCR-transduced CD8+ T cells upregulating 4-1BB expression following co-culture was measured by FACs. The results are shown in FIGS. 7A-7H. The results showed that 4286 TCR 10-1, 4286 TCR 10-2, 4286 TCR 10-3, and 4286 TCR 10-4 avidly and specifically recognized mutated 9-mer LLVDLAEEL (SEQ ID NO: 142) peptide and mutated 10-mer RLLVDLAEEL (SEQ ID NO: 143) peptide, as measured by IFN-γ secretion and 4-1BB upregulation.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplarylanguage (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate. and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated or purified T-cell receptor (TCR) having antigenic specificity for a mutated amino acid sequence encoded by mutated cyclin-dependent kinase inhibitor 2A (CDKN2A), wherein the TCR comprises the amino acid sequence(s) of:
(a) all of SEQ ID NOs: 1-3;
(b) all of SEQ ID NOs: 4-6;
(c) all of SEQ ID NOs: 1-6;
(d) all of SEQ ID NOs: 11-13;
(e) all of SEQ ID NOs: 14-16;
(f) all of SEQ ID NOs: 11-16;
(g) all of SEQ ID NOs: 21-23;
(h) all of SEQ ID NOs: 24-26;
(i) all of SEQ ID NOs: 21-26;
(j) all of SEQ ID NO: 31-33;
(k) all of SEQ ID NO: 34-36;
(l) all of SEQ ID NO: 31-36;
(m) all of SEQ ID NO: 41-43;
(n) all of SEQ ID NO: 44-46;
(o) all of SEQ ID NO: 41-46;
(p) all of SEQ ID NO: 51-53;
(q) all of SEQ ID NO: 54-56;
(r) all of SEQ ID NO: 51-56;
(s) all of SEQ ID NO: 61-63;
(t) all of SEQ ID NO: 64-66; or
(u) all of SEQ ID NO: 61-66.

2-7. (canceled)

8. The TCR according to claim 1, comprising the amino acid sequence(s) of:
(i) SEQ ID NO: 7,
(ii) SEQ ID NO: 8,
(iii) SEQ ID NO: 9,
(iv) SEQ ID NO: 10,
(v) both of SEQ ID NOs: 7 and 8,
(vi) both of SEQ ID NOs: 9 and 10,
(vii) SEQ ID NO: 17,
(viii) SEQ ID NO: 18,
(ix) SEQ ID NO: 19,
(x) SEQ ID NO: 20,
(xi) both of SEQ ID NOs: 17 and 18,
(xii) both of SEQ ID NOs: 19 and 20,
(xiii) SEQ ID NO: 27,
(xiv) SEQ ID NO: 28,
(xv) SEQ ID NO: 29,
(xvi) SEQ ID NO: 30,
(xvii) both of SEQ ID NOs: 27 and 28,
(xviii) both of SEQ ID NOs: 29 and 30,
(xix) SEQ ID NO: 37,
(xx) SEQ ID NO: 38,
(xi) SEQ ID NO: 39,
(xxii) SEQ ID NO: 40,
(xxiii) both of SEQ ID NOs: 37 and 38,
(xxiv) both of SEQ ID NOs: 39 and 40,
(xxv) SEQ ID NO: 47,
(xxvi) SEQ ID NO: 48,
(xxvii) SEQ ID NO: 49,
(xxviii) SEQ ID NO: 50,
(xxix) both of SEQ ID NOs: 47 and 48,
(xxx) both of SEQ ID NOs: 49 and 50,
(xxxi) SEQ ID NO: 57,
(xxxii) SEQ ID NO: 58,
(xxxiii) SEQ ID NO: 59,
(xxxiv) SEQ ID NO: 60,
(xxxv) both of SEQ ID NOs: 57 and 58,
(xxxvi) both of SEQ ID NOs: 59 and 60,
(xxxvii) SEQ ID NO: 67,
(xxxviii) SEQ ID NO: 68,
(xxxix) SEQ ID NO: 69,
(xl) SEQ ID NO: 70,
(xli) both of SEQ ID NOs: 67 and 68, or
(xlii) both of SEQ ID NOs: 69 and 70.

9. The TCR of claim 1, further comprising;
(a) an α chain constant region comprising the amino acid sequence of SEQ ID NO: 71, wherein;
(i) X at position 48 of SEQ ID NO: 71 is Thr or Cys;
(ii) X at position 112 of SEQ ID NO: 71 is Ser, Ala, Val, Leu, Ile, Pro, Phe, Met, or Trp;
(iii) X at position 114 of SEQ ID NO: 71 is Met, Ala, Val, Leu, Ile, Pro, Phe, or Trp; and
(iv) X at position 115 of SEQ ID NO: 71 is Gly, Ala, Val, Leu, Ile, Pro, Phe, Met, or Trp;
(b) a β chain constant region comprising the amino acid sequence of SEQ ID NO: 72, wherein X at position 57 of SEQ ID NO: 72 is Ser or Cys; or
(c) both (a) and (b).

10. (canceled)

11. An isolated or purified polypeptide comprising a functional portion of the TCR of claim 1, wherein the functional portion comprises the amino acid sequences of:
(a) all of SEQ ID NOs: 1-3;
(b) all of SEQ ID NOs: 4-6;
(c) all of SEQ ID NOs: 1-6;
(d) all of SEQ ID NOs: 11-13;
(e) all of SEQ ID NOs: 14-16;
(f) all of SEQ ID NOs: 11-16;
(g) all of SEQ ID NOs: 21-23;
(h) all of SEQ ID NOs: 24-26;
(i) all of SEQ ID NOs: 21-26;
(j) all of SEQ ID NO: 31-33;
(k) all of SEQ ID NO: 34-36;
(l) all of SEQ ID NO: 31-36;
(m) all of SEQ ID NO: 41-43;
(n) all of SEQ ID NO: 44-46;
(o) all of SEQ ID NO: 41-46;
(p) all of SEQ ID NO: 51-53;
(q) all of SEQ ID NO: 54-56;
(r) all of SEQ ID NO: 51-56;
(s) all of SEQ ID NO: 61-63;
(t) all of SEQ ID NO: 64-66; or
(u) all of SEQ ID NO: 61-66.

12. The isolated or purified polypeptide according to claim 11, wherein the functional portion comprises the amino acid sequence(s) of:
(i) SEQ ID NO: 7,
(ii) SEQ ID NO: 8,
(iii) SEQ ID NO: 9,
(iv) SEQ ID NO: 10,
(v) both of SEQ ID NOs: 7 and 8,
(vi) both of SEQ ID NOs: 9 and 10,
(vii) SEQ ID NO: 17,
(viii) SEQ ID NO: 18,
(ix) SEQ ID NO: 19,
(x) SEQ ID NO: 20,
(xi) both of SEQ ID NOs: 17 and 18,
(xii) both of SEQ ID NOs: 19 and 20,
(xiii) SEQ ID NO: 27,
(xiv) SEQ ID NO: 28,
(xv) SEQ ID NO: 29,
(xvi) SEQ ID NO: 30,
(xvii) both of SEQ ID NOs: 27 and 28,
(xviii) both of SEQ ID NOs: 29 and 30,
(xix) SEQ ID NO: 37,
(xx) SEQ ID NO: 38,
(xi) SEQ ID NO: 39,
(xxii) SEQ ID NO: 40,
(xxiii) both of SEQ ID NOs: 37 and 38,
(xxiv) both of SEQ ID NOs: 39 and 40,
(xxv) SEQ ID NO: 47,
(xxvi) SEQ ID NO: 48,
(xxvii) SEQ ID NO: 49,
(xxviii) SEQ ID NO: 50,
(xxix) both of SEQ ID NOs: 47 and 48,
(xxx) both of SEQ ID NOs: 49 and 50,
(xxxi) SEQ ID NO: 57,
(xxxii) SEQ ID NO: 58,
(xxxiii) SEQ ID NO: 59,
(xxxiv) SEQ ID NO: 60,
(xxxv) both of SEQ ID NOs: 57 and 58,
(xxxvi) both of SEQ ID NOs: 59 and 60,
(xxxvii) SEQ ID NO: 67,
(xxxviii) SEQ ID NO: 68,
(xxxix) SEQ ID NO: 69,
(xl) SEQ ID NO: 70,
(xli) both of SEQ ID NOs: 67 and 68, or
(xlii) both of SEQ ID NOs: 69 and 70.

13. The isolated or purified polypeptide of claim 11, further comprising;
(a) the amino acid sequence of SEQ ID NO: 71, wherein;
(i) X at position 48 of SEQ ID NO: 71 is Thr or Cys;
(ii) X at position 112 of SEQ ID NO: 71 is Ser, Ala, Val, Leu, Ile, Pro, Phe, Met, or Trp;
(iii) X at position 114 of SEQ ID NO: 71 is Met, Ala, Val, Leu, Ile, Pro, Phe, or Trp; and
(iv) X at position 115 of SEQ ID NO: 71 is Gly, Ala, Val, Leu, Ile, Pro, Phe, Met, or Trp;
(b) the amino acid sequence of SEQ ID NO: 72, wherein X at position 57 of SEQ ID NO: 72 is Ser or Cys; or
(c) both (a) and (b).

14. (canceled)

15. An isolated or purified protein, comprising;
(a) a first polypeptide chain comprising the amino acid sequences of SEQ ID NOs: 1-3 and a second polypeptide chain comprising the amino acid sequences of SEQ ID NOs: 4-6;
(b) a first polypeptide chain comprising the amino acid sequences of SEQ ID NOs: 11-13 and a second polypeptide chain comprising the amino acid sequences of SEQ ID NOs: 14-16;
(c) a first polypeptide chain comprising the amino acid sequences of SEQ ID NOS; 21-23 and a second polypeptide chain comprising the amino acid sequences of SEQ ID NOs: 24-26;
(d) a first polypeptide chain comprising the amino acid sequences of SEQ ID NOs: 31-33 and a second polypeptide chain comprising the amino acid sequences of SEQ ID NOs: 34-36;
(e) a first polypeptide chain comprising the amino acid sequences of SEQ ID NOs: 41-43 and a second polypeptide chain comprising the amino acid sequences of SEQ ID NOs: 44-46;
(f) a first polypeptide chain comprising the amino acid sequences of SEQ ID NOs: 51-53 and a second polypeptide chain comprising the amino acid sequences of SEQ ID NOs: 54-56; or
(g) a first polypeptide chain comprising the amino acid sequences of SEQ ID NOS; 61-63 and a second polypeptide chain comprising the amino acid sequences of SEQ ID NOS; 64-66.

16. The isolated or purified protein according to claim 15, wherein;
(i) the first polypeptide chain comprises the amino acid sequence of SEQ ID NO: 7 and the second polypeptide chain comprises the amino acid sequence of SEQ ID NO: 8;
(ii) the first polypeptide chain comprises the amino acid sequence of SEQ ID NO: 9 and the second polypeptide chain comprises the amino acid sequence of SEQ ID NO: 10;
(iii) the first polypeptide chain comprises the amino acid sequence of SEQ ID NO: 17 and the second polypeptide chain comprises the amino acid sequence of SEQ ID NO: 18;
(iv) the first polypeptide chain comprises the amino acid sequence of SEQ ID NO: 19 and the second polypeptide chain comprises the amino acid sequence of SEQ ID NO: 20;
(v) the first polypeptide chain comprises the amino acid sequence of SEQ ID NO: 27 and the second polypeptide chain comprises the amino acid sequence of SEQ ID NO: 28;
(vi) the first polypeptide chain comprises the amino acid sequence of SEQ ID NO: 29 and the second polypeptide chain comprises the amino acid sequence of SEQ ID NO: 30;
(vii) the first polypeptide chain comprises the amino acid sequence of SEQ ID NO: 37 and the second polypeptide chain comprises the amino acid sequence of SEQ ID NO: 38;
(viii) the first polypeptide chain comprises the amino acid sequence of SEQ ID NO: 39 and the second polypeptide chain comprises the amino acid sequence of SEQ ID NO: 40;
(ix) the first polypeptide chain comprises the amino acid sequence of SEQ ID NO: 47 and the second polypeptide chain comprises the amino acid sequence of SEQ ID NO: 48;
(x) the first polypeptide chain comprises the amino acid sequence of SEQ ID NO: 49 and the second polypeptide chain comprises the amino acid sequence of SEQ ID NO: 50;
(xi) the first polypeptide chain comprises the amino acid sequence of SEQ ID NO: 57 and the second polypeptide chain comprises the amino acid sequence of SEQ ID NO: 58;
(xii) the first polypeptide chain comprises the amino acid sequence of SEQ ID NO: 59 and the second polypeptide chain comprises the amino acid sequence of SEQ ID NO: 60;
(xiii) the first polypeptide chain comprises the amino acid sequence of SEQ ID NO: 67 and the second polypeptide chain comprises the amino acid sequence of SEQ ID NO: 68; or
(xiv) the first polypeptide chain comprises the amino acid sequence of SEQ ID NO: 69 and the second polypeptide chain comprises the amino acid sequence of SEQ ID NO: 70.

17. The isolated or purified protein of claim 15, 45 of 16, wherein;
(a) the first polypeptide chain further comprises the amino acid sequence of SEQ ID NO: 71, wherein;
(i) X at position 48 of SEQ ID NO: 71 is Thr or Cys;
(ii) X at position 112 of SEQ ID NO: 71 is Ser, Ala, Val, Leu, Ile, Pro, Phe, Met, or Trp;
(iii) X at position 114 of SEQ ID NO: 71 is Met, Ala, Val, Leu, Ile, Pro, Phe, or Trp; and
(iv) X at position 115 of SEQ ID NO: 71 is Gly, Ala, Val, Leu, Ile, Pro, Phe, Met, or Trp;
(b) the second polypeptide chain further comprises the amino acid sequence of SEQ ID NO: 72, wherein X at position 57 of SEQ ID NO: 72 is Ser or Cys; or
(c) both (a) and (b).

18. (canceled)

19. A bispecific engager TCR fusion protein comprising (i) the TCR according to claim 1 and (ii) an anti-CD3 effector.

20. An isolated or purified nucleic acid comprising a nucleotide sequence encoding the TCR according to claim 1.

21. An isolated or purified nucleic acid comprising, from 5′ to 3′, a first nucleic acid sequence and a second nucleotide sequence, wherein the first and second nucleotide sequence, respectively, encode the amino sequences of SEQ ID NOs: 7 and 8; 8 and 7; 9 and 10; 10 and 9; 17 and 18; 18 and 17; 19 and 20; 20 and 19; 27 and 28; 28 and 27; 29 and 30; 30 and 29; 37 and 38; 38 and 37; 39 and 40; 40 and 39; 47 and 48; 48 and 47; 49 and 50; 50 and 49; 57 and 58; 58 and 57; 59 and 60; 60 and 59; 67 and 68; 68 and 67; 69 and 70; 70 and 69; 77 and 78; 78 and 77; 79 and 80; 80 and 79; 81 and 82; 82 and 81; 83 and 84; 84 and 83; 85 and 86; 86 and 85; 87 and 88; 88 and 87; 89 and 90; 90 and 89; 91 and 92; 92 and 91; 93 and 94; 94 and 93; 95 and 96; 96 and 95; 97 and 98; 98 and 97; 99 and 100; 100 and 99; 101 and 102; 102 and 101; 103 and 104; 104 and 103; 105 and 106; 106 and 105; 107 and 108; 108 and 107; 109 and 110; 111 and 112; 112 and 111; 113 and 114; 114 and 113; 115 and 116; 116 and 115; 117 and 118; 118 and 117; 119 and 120; 120 and 119; 121 and 122; 122 and 121; 123 and 124; 124 and 123; 125 and 126; 126 and 125; 127 and 128; 128 and 127; 129 and 130; 130 and 129; 131 and 132; or 132 and 131.

22-23. (canceled)

24. A recombinant expression vector comprising the nucleic acid according to claim 20.

25-27. (canceled)

28. A method of producing a host cell expressing a TCR that has antigenic specificity for the peptide of AVCPWTWLR (SEQ ID NO: 141), LLVDLAEEL (SEQ ID NO: 142), or RLLVDLAEEL (SEQ ID NO: 143), the method comprising contacting a cell in vitro with the recombinant expression vector according to claim 24 under conditions that allow introduction of the recombinant expression vector into the cell.

29. An isolated or purified host cell comprising the nucleic acid according to claim 20 or an isolated or purified population of cells comprising the host cell.

30-32. (canceled)

33. A method of producing a T cell receptor (TCR), the method comprising culturing the host cell according to claim 29, so that the TCR is produced.

34. A pharmaceutical composition comprising (a) the population of cells according to claim 29 and (b) a pharmaceutically acceptable carrier.

35. A method of detecting the presence of cancer in mammal, the method comprising;
(a) contacting a sample comprising cells of the cancer with the TCR according to claim 1, thereby forming a complex; and
(b) detecting the complex,
wherein detection of the complex is indicative of the presence of cancer in the mammal.

36-39. (canceled)

40. A method of inducing an immune response against cancer in a mammal, the method comprising administering to the mammal the population of cells according to claim 29 in an amount effective to induce the immune response against cancer in the mammal.

41. A method of treating or preventing cancer in a mammal, the method comprising administering to the mammal the population of cells according to claim 29 in an amount effective to treat or prevent cancer in the mammal.
